(19) United States
(12) Patent Application Publication
(10) Pub. No.: US 2024/0067971 A1
(43) Pub. Date: Feb. 29, 2024

(54) Target Ligand

(71) Applicant: NANOPEPTIDE (QINGDAO) BIOTECHNOLOGY LTD., Qingdao (CN)

(72) Inventors: Zhaogui Liu, Qingdao (CN); Ping Chen, Qingdao (CN); Jieting Zhang, Qingdao (CN); Rui Wang, Qingdao (CN); Juan Xu, Qingdao (CN); Zhongguo Fu, Qingdao (CN); Hailin Zhang, Qingdao (CN); Pu Chen, Qingdao (CN)

(21) Appl. No.: 18/130,418

(22) Filed: Apr. 3, 2023

Related U.S. Application Data
(63) Continuation of application No. 18/092,202, filed on Dec. 30, 2022.
Application No. 18/092,202 is a continuation-in-part of application No. PCT/CN2021/122118, filed on Sep. 30, 2021.

(30) Foreign Application Priority Data
Sep. 30, 2020 (CN) 202011061038.1
Jan. 5, 2021 (CN) 202110008013.3
Apr. 13, 2021 (CN) 202110397429.9

(51) Int. Cl. C12N 15/113

(52) U.S. Cl. CPC C12N 15/1136; C12N 2310/11; C12N 2310/14; C12N 2310/315; C12N 2310/3181; C12N 2310/321; C12N 2310/3233; C12N 2310/33; C12N 2310/3341

(57) ABSTRACT

The present disclosure relates to the technical field of genetic engineering, in particular to a target ligand. The target ligand provided by the present disclosure forms a siRNA conjugate with a specific small interfering RNA sequence, which targets to an angiopoietin like 3 (ANGPTL3), and reduce the expression quantity of an ANGPTL3 protein by degrading a transcript of an ANGPTL3 gene in a cell. Therefore, the siRNA conjugate formed by the target ligand provided by the present disclosure may be used to prevent and/or treat a dyslipidemia disease.

### CROSS-REFERENCE TO RELATED APPLICATION

The present application is Continuation Application of U.S. patent application Ser. No. 18/092,202 filed on Dec. 30, 2022, which is a Continuation-In-Part application of PCT Application No. PCT/CN2021/122118 filed on Sep. 30, 2021, which claims the benefit of Chinese Patent Application Nos. 202011061038.1 filed on Sep. 30, 2020, 202110008013.3 filed on Jan. 5, 2021 and 202110397429.9 filed on Apr. 13, 2021. The contents of all of the aforementioned applications are incorporated by reference herein in their entirety.

### REFERENCE TO SEQUENCE LISTING

The Substitute Sequence Listing XML file is submitted to replace the previously sequence listing XML file submitted via the USPTO Patent Center, with a file name of “Substitute_Sequence_Listing_RONDA-22020-USCIP.xml”, a creation date of Jul. 17, 2023, and a size of 268 KB. The Substitute Sequence Listing XML file is a part of the specification and is incorporated in its entirety by reference herein.

### TECHNICAL FIELD

The present disclosure relates to the technical field of genetic engineering, in particular to a new compound that may be used as a target ligand.

### BACKGROUND

Hyperlipidemia, also known as dyslipidemia, is a systemic disease with abnormal fat metabolism or operation, which makes plasma lipids higher than a normal value. The clinical manifestations of the dyslipidemia mainly include two aspects: (1) xanthoma caused by lipid deposition in dermis; and (2) atherosclerosis caused by the lipid deposition in vascular endothelium, generating a coronary heart disease and a peripheral vascular disease and the like. According to the survey, about 10% to 20% of adults have the elevated blood total cholesterol (TC) or triglycerides (TG), and even nearly 10% of children have the elevated blood lipids. Existing drugs for the dyslipidemia mainly include statins, cholesterol absorption inhibitors, resins, probucol, fibrates, niacin and derivatives thereof.

The angiopoietin like protein 3 (ANGPTL3, NM_014495.4) is a secreted protein mainly expressed in a liver cell. It is indicated from existing researches that ANGPTL3 is a key regulatory factor of low-density lipoprotein cholesterol (LDL-C), high-density lipoprotein cholesterol (HDL-C) and triglyceride metabolism, and has a variety of potential action nodes. The loss of function mutation of ANGPTL3 may lead to the reduction of LDL-C, very low-density lipoprotein cholesterol (VLDL-C), HDL-C and triglyceride (TG), thus the risk of cardiovascular diseases based on genome wide association study (GWAS) is reduced, and there are no known adverse phenotypes of genetic defects. Therefore, the inhibition of the activity of ANGPTL3 may effectively prevent or treat the dyslipidemia.

Most of existing technologies use an ANGPTL3 antibody to inhibit its activity. A Chinese patent with the application number of CN201280038908.0 discloses a completely humanized antibody or an antigen-binding fragment of a human antibody, it specifically binds to a human angiopoietin-like protein 3 (hANGPTL3) and inhibits or interferes at least one of its activities. The human anti-hANGPTL3 antibody may be used to treat diseases or disorders related to ANGPTL3, such as hyperlipidemia, hyperlipoproteinemia and dyslipidemia, including hypertriglyceridemia, hypercholesterolemia, chylomicroxemia and the like.

A Chinese patent with the application number of CN201780026147. X discloses a method for treating a patient suffered from familial hypercholesterolemia, including HeFH and HoFH. By administering a therapeutically effective amount of the specific ANGPTL3-binding antibody or its antigen-binding fragment, it is combined with other drugs, and at least one lipid parameter of the patient is reduced. It may be used to treat hypercholesterolemia, hyperlipidemia, hyperlipoproteinemia and dyslipidemia, including the hypertriglyceridemia and the chylomicroxemia.

Compared with antibodies, the siRNA drug has the advantages of rich candidate targets, short research and development cycle, and high clinical development success rate. Therefore, it has the more advantages in the use of drugs for chronic diseases. However, the defects of the siRNA drug itself (such as: poor stability, and difficulty in transmembrane) limit its clinical applications. The structural transformation of siRNA or the construction of conjugates may improve the drug ability of siRNA to a certain extent. To explore constituent molecules of the conjugates constructed is the only way which must be passed to develop the siRNA drug for the dyslipidemia, and is also an urgent problem to be solved.

### SUMMARY

The present disclosure aims to solve at least one of technical problems in a related technology to a certain extent. For this reason, a purpose of the present disclosure is to provide a siRNA for inhibiting expression of ANGPTL3. The inventor of the present disclosure targets to ANGPTL3 by designing an appropriate specific small interfering RNA sequence and combining it with a target ligand to form a siRNA conjugate, and reduce the expression of an ANGPTL3 protein by degrading a transcript of an ANGPTL3 gene in a cell. Therefore, the siRNA provided in the present disclosure may be used to prevent and/or treat a dyslipidemia disease.

For this reason, on the one hand, the present disclosure provides a siRNA. According to an embodiment of the present disclosure, the siRNA includes a sense chain and an antisense chain, and the antisense chain includes a complementary region complementary-paired to the sense chain, herein the sense chain is selected from a nucleotide sequence that is not more than 5 nucleotides different from a nucleotide sequence of each chain in SEQ ID NO: 1-SEQ ID NO: 154, and the antisense chain is selected from a nucleotide sequence that is not more than 5 nucleotides different from a nucleotide sequence of each chain in SEQ ID NO: 155-SEQ ID NO: 308.

The inventor of the present disclosure specifically reduces the synthesis of ANGPTL3 by the liver cell by designing the appropriate small interfering RNA (siRNA) sequence, while the off-target effect is avoided. siRNA, by forming a RNA-induced silencing complex (RISC), is complementary-paired with a mRNA sequence of a target gene (ANGPTL3 gene) to degrade mRNA of the target gene so as to inhibit the expression of the target gene, and then reduce the levels of LDL-C, VLDL-C, HDL-C and TG.

The siRNA according to an embodiment of the present disclosure may also have at least one of the following additional technical features.

The present disclosure further provides a siRNA, and the siRNA is selected from any pair of siRNA in any one of the following groups.

(1) It may specifically target to the 60-80-th nucleotides of the ANGPTL3gene sequence; preferably, the sense chain of the siRNA is selected from SEQ ID NO: 10, and the antisense chain is selected from SEQ ID NO: 165.

(2) It may specifically target to the 107-133-th nucleotides of the ANGPTL3genesequence; preferably, the sense chain of the siRNA is selected from SEQ ID NO: 17, and the antisense chain is selected from SEQ ID NO: 171, or the sense chain of the siRNA is selected from SEQ ID NO: 18, and the antisense chain is selected from SEQ ID NO: 172.

(3) It may specifically target to the 163-187-th nucleotides of the ANGPTL3genesequence; preferably, the sense chain of the siRNA is selected from SEQ ID NO: 19, and the antisense chain is selected from SEQ ID NO: 173.

(4) It may specifically target to the 304-388-th nucleotides of the ANGPTL3genesequence, and preferably, it may specifically target to the 304-359-th nucleotides of the ANGPTL3 sequence; more preferably, the sense chain of the siRNA is selected from SEQ ID NO: 27, and the antisense chain is selected from SEQ ID NO: 181,
or, the sense chain of the siRNA is selected from SEQ ID NO: 29, and the antisense chain is selected from SEQ ID NO: 183,
or, the sense chain of the siRNA is selected from SEQ ID NO: 31, and the antisense chain is selected from SEQ ID NO: 185,
or, the sense chain of the siRNA is selected from SEQ ID NO: 32, and the antisense chain is selected from SEQ ID NO: 186,
or, the sense chain of the siRNA is selected from SEQ ID NO: 35, and the antisense chain is selected from SEQ ID NO: 189,
or, the sense chain of the siRNA is selected from SEQ ID NO: 36, and the antisense chain is selected from SEQ ID NO: 190.

(5) It may specifically target to the 430-459-th nucleotides of the ANGPTL3genesequence; preferably, the sense chain of the siRNA is selected from SEQ ID NO: 43, and the antisense chain is selected from SEQ ID NO: 197,
or, the sense chain of the siRNA is selected from SEQ ID NO: 44, and the antisense chain is selected from SEQ ID NO: 198.

(6) It may specifically target to the 1360-1430-th nucleotides of the ANGPTL3genesequence, and preferably, it may specifically target to the 1397-1430-th nucleotides of the ANGPTL3genesequence; more preferably, the sense chain of the siRNA is selected from SEQ ID NO: 145, and the antisense chain is selected from SEQ ID NO: 299,
or, the sense chain of the siRNA is selected from SEQ ID NO: 150, and the antisense chain is selected from SEQ ID NO: 304,
or, the sense chain of the siRNA is selected from SEQ ID NO: 151, and the antisense chain is selected from SEQ ID NO: 305,
or, the sense chain of the siRNA is selected from SEQ ID NO: 152, and the antisense chain is selected from SEQ ID NO: 306,
or, the sense chain of the siRNA is selected from SEQ ID NO: 154, and the antisense chain is selected from SEQ ID NO: 308.

According to an embodiment of the present disclosure, the siRNA includes at least one modified nucleotide.

Optionally, the modified nucleotide is selected from at least one of the following:
a 5′-thiophosphate based nucleotide, a 5-methylcytosine nucleotide, a 2′-O-methyl modified nucleotide, a 2′-O-2-methoxyethyl modified nucleotide, a 2′-fluoro modified nucleotide, a 3′-nitrogen substituted modified nucleotide, a 2′-deoxy-2′-fluoro modified nucleotide, a 2′-deoxy modified nucleotide, a locked nucleotide, a de-base nucleotide, a 2′-amino modified nucleotide, a morpholino nucleotide, a polypeptide nucleotide, an amino phosphate, and a nucleotide including a nonnatural base.

According to an embodiment of the present disclosure, the length of the complementary region is at least 17 bp.

Optionally, the length of the complementary region is 18-21 bp.

Optionally, the length of the complementary region is 19 bp.

According to an embodiment of the present disclosure, the lengths of the sense chain and the antisense chain in the siRNA are not more than 25 bp.

Optionally, the lengths of the sense chain and the antisense chain in the siRNA are 18-25 bp.

Optionally, the lengths of the sense chain and the antisense chain in the siRNA are 21 bp.

According to an embodiment of the present disclosure, the bases in the sense chain and the antisense chain of the siRNA may be complementary-paired one-to-one, or may be dislocated for several bases, but have at least 17 bp of the complementary region.

On the other hand, the present disclosure provides a siRNA conjugate, and the siRNA conjugate includes the previously described siRNA and a target ligand, herein the siRNA is covalently linked with the target ligand.

Preferably, the target ligand is linked to the sense chain in the siRNA.

More preferably, the target ligand is linked with a 5′-end of the sense chain in the siRNA by a thiophosphate bond.

According to an embodiment of the present disclosure, the target ligand includes at least one N-acetyl-galactosamine (GalNAC).

According to an embodiment of the present disclosure, the target ligand is a GalNAC target compound.

According to an embodiment of the present disclosure, the GalNAC target compound is 1043, 1046 and 1048, and its structure is shown as follows:

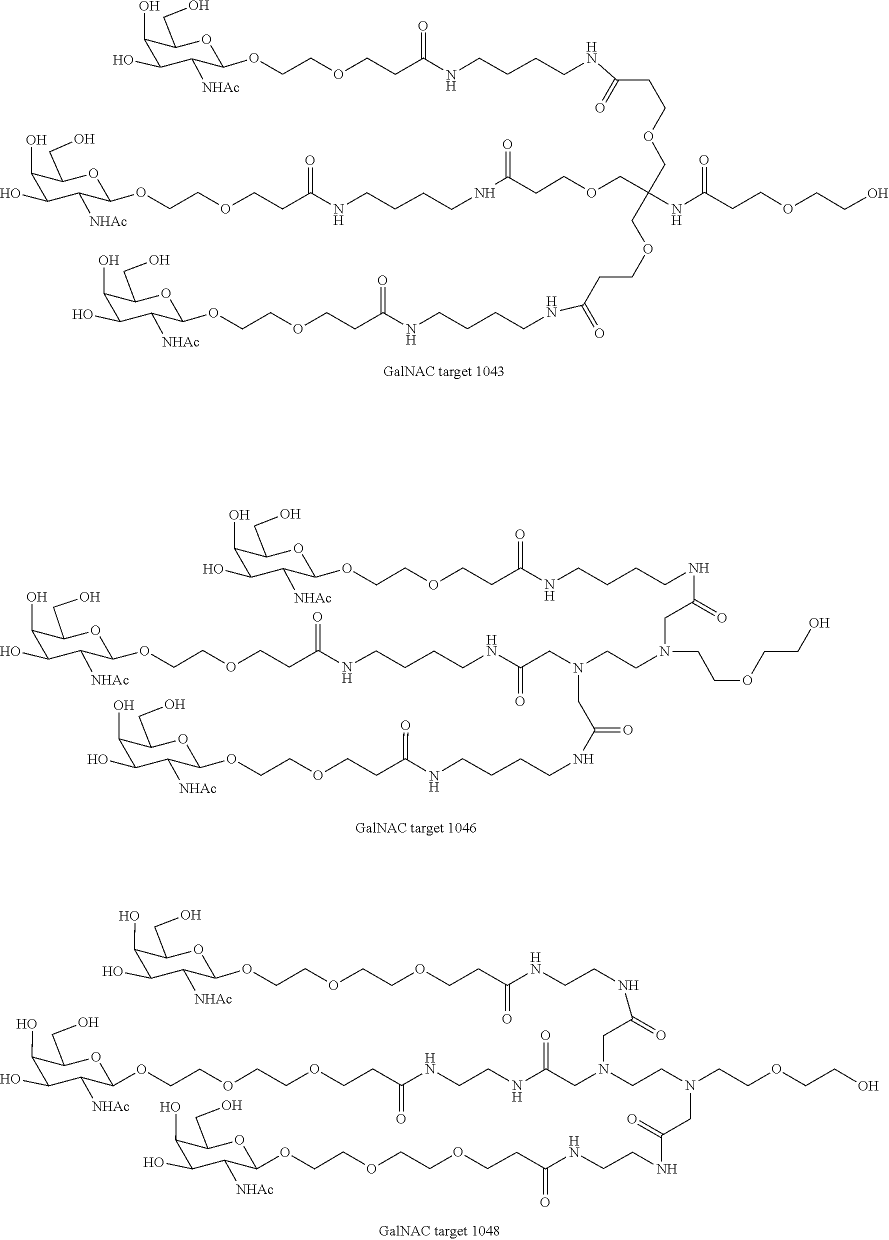 [SMILES: CC(=O)NC1C(OCCOCCC(=O)NCCCCNC(=O)CCOCC(COCCC(=O)NCCCCNC(=O)CCOCCOC2OC(CO)C(O)C(O)C2NC(C)=O)(COCCC(=O)NCCCCNC(=O)CCOCCOC2OC(CO)C(O)C(O)C2NC(C)=O)NC(=O)CCOCCO)OC(CO)C(O)C1O ; CC(=O)NC1C(OCCOCCC(=O)NCCCCNC(=O)CN(CCOCCO)CCN(CC(=O)NCCCCNC(=O)CCOCCOC2OC(CO)C(O)C(O)C2NC(C)=O)CC(=O)NCCCCNC(=O)CCOCCOC2OC(CO)C(O)C(O)C2NC(C)=O)OC(CO)C(O)C1O ; CC(=O)NC1C(OCCOCCOCCC(=O)NCCNC(=O)CN(CCOCCO)CCN(CC(=O)NCCNC(=O)CCOCCOCCOC2OC(CO)C(O)C(O)C2NC(C)=O)CC(=O)NCCNC(=O)CCOCCOCCOC2OC(CO)C(O)C(O)C2NC(C)=O)OC(CO)C(O)C1O]

According to an embodiment of the present disclosure, the target ligand is linked to the sense chain in the siRNA.

On the other hand, the present disclosure provides a pharmaceutical composition. According to an embodiment of the present disclosure, the pharmaceutical composition includes the previously described siRNA and/or the previously described siRNA conjugate, and optionally, the pharmaceutical composition further includes a pharmaceutically acceptable excipient.

Therefore, the pharmaceutical composition according to the embodiment of the present disclosure may be used to inhibit the synthesis of ANGPTL3 by the cells, thereby the levels of LDL-C, VLDL-C, HDL-C and TG are reduced, as to prevent and/or treat hyperlipidemia and hypertriglyceridemia.

On the other hand, the present disclosure provides a compound.

The compound has any one of the following structures:

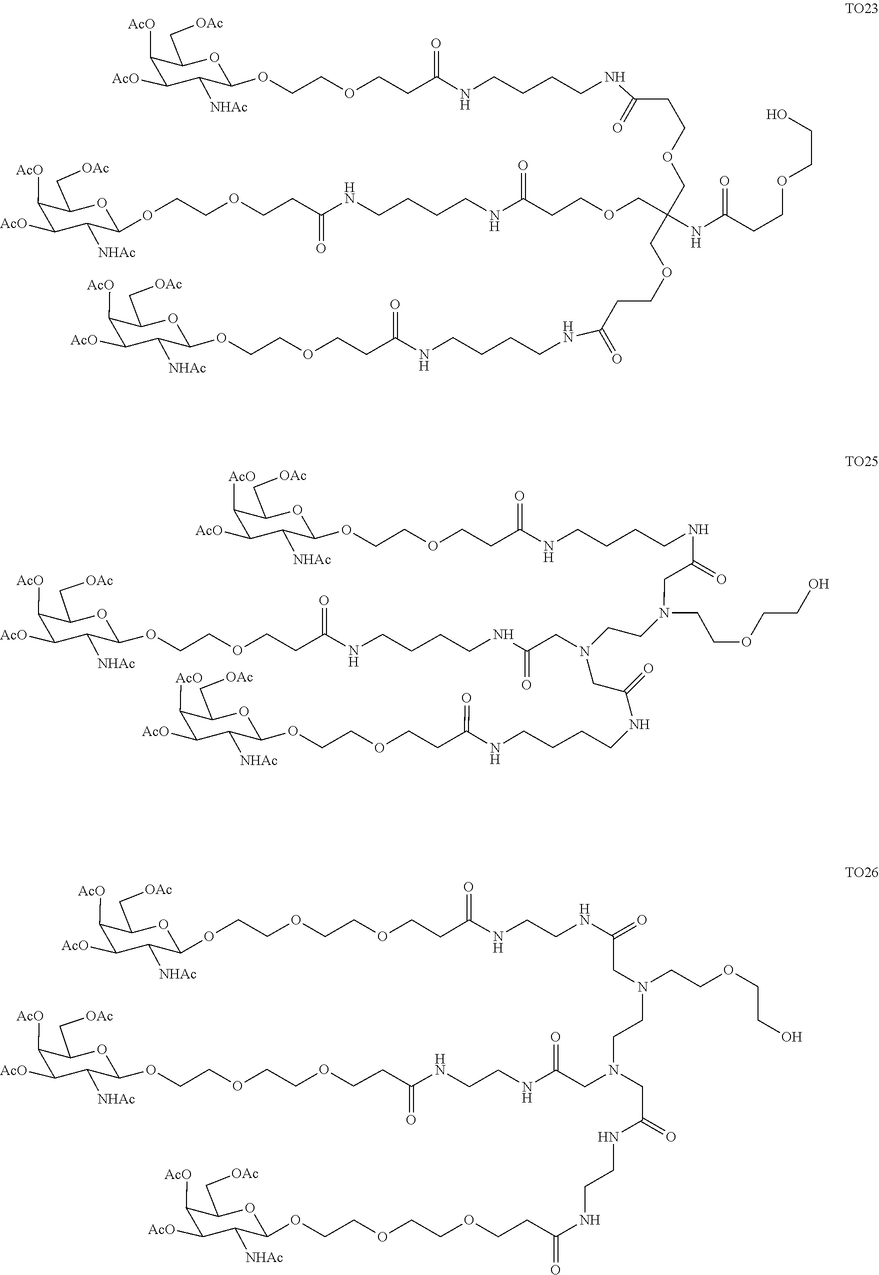

The structures of the above compounds obtained after a hydroxyl protecting group is removed are as follows:

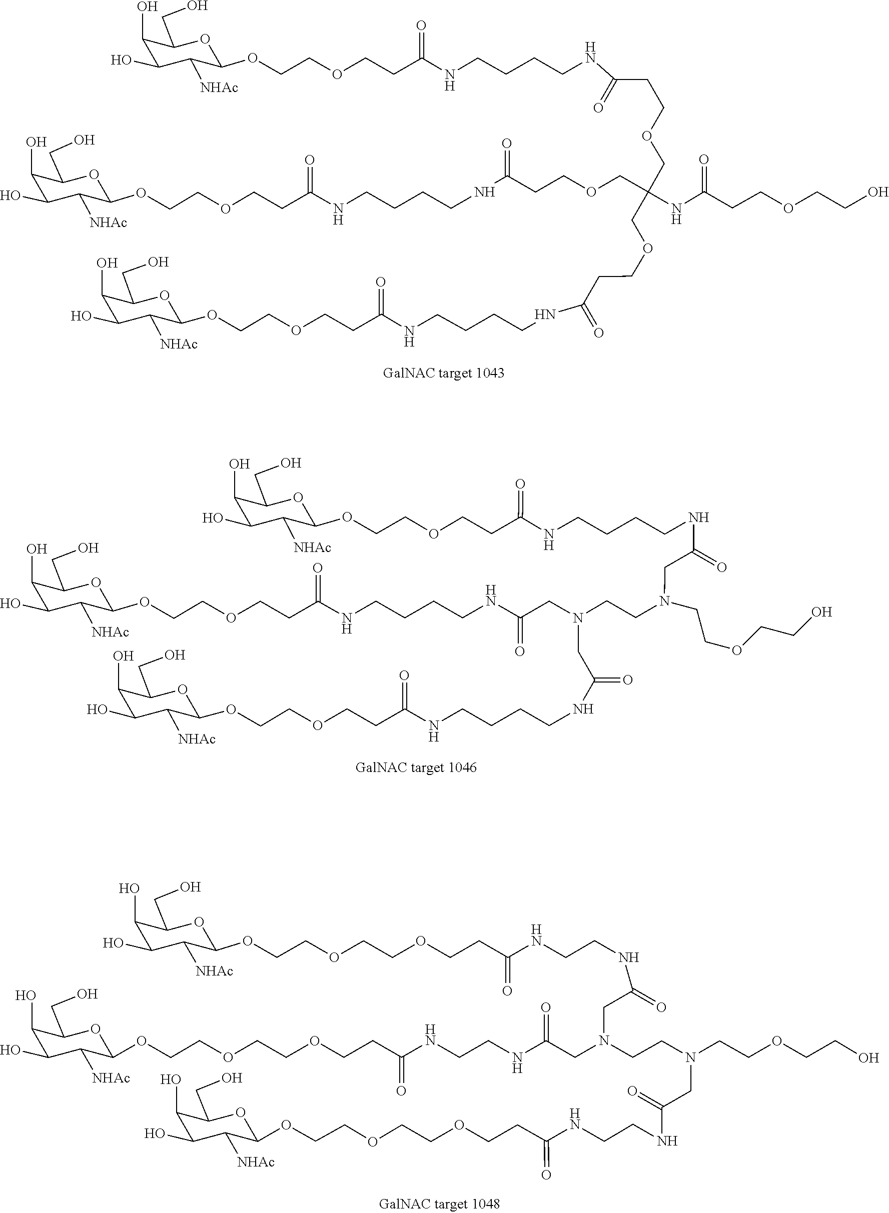 [SMILES: CC(=O)NC1C(OCCOCCC(=O)NCCCCNC(=O)CCOCC(COCCC(=O)NCCCCNC(=O)CCOCCOC2OC(CO)C(O)C(O)C2NC(C)=O)(COCCC(=O)NCCCCNC(=O)CCOCCOC2OC(CO)C(O)C(O)C2NC(C)=O)NC(=O)CCOCCO)OC(CO)C(O)C1O ; CC(=O)NC1C(OCCOCCC(=O)NCCCCNC(=O)CN(CCOCCO)CCN(CC(=O)NCCCCNC(=O)CCOCCOC2OC(CO)C(O)C(O)C2NC(C)=O)CC(=O)NCCCCNC(=O)CCOCCOC2OC(CO)C(O)C(O)C2NC(C)=O)OC(CO)C(O)C1O ; CC(=O)NC1C(OCCOCCOCCC(=O)NCCNC(=O)CN(CCOCCO)CCN(CC(=O)NCCNC(=O)CCOCCOCCOC2OC(CO)C(O)C(O)C2NC(C)=O)CC(=O)NCCNC(=O)CCOCCOCCOC2OC(CO)C(O)C(O)C2NC(C)=O)OC(CO)C(O)C1O]

On the other hand, the present invention provides an application of the aforementioned compounds in preparation of a siRNA conjugate.

The compounds of TO23, TO25 and TO26 firstly form intermediates and then covalently link with siRNA, and the intermediates are selected from:

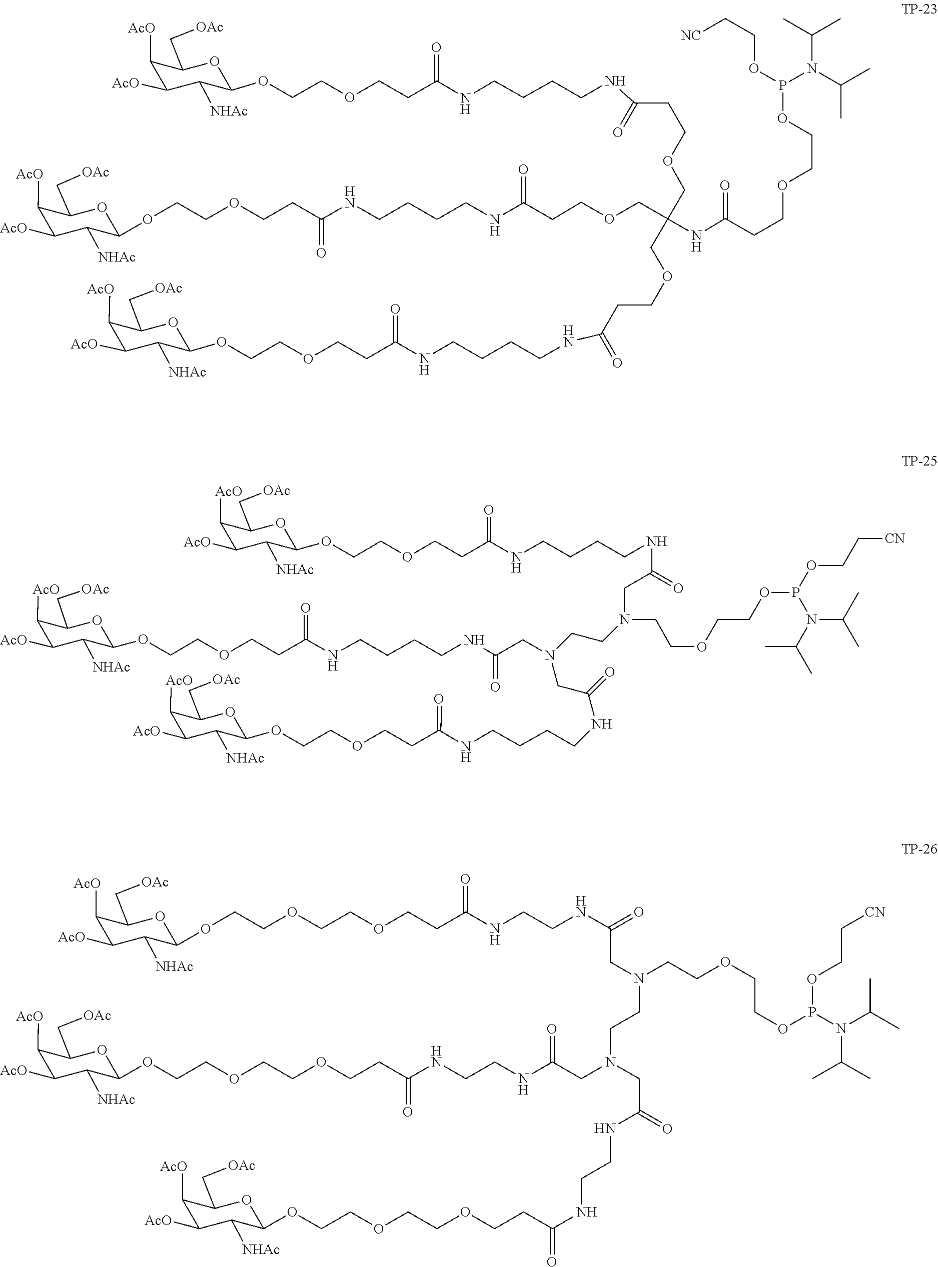

The present disclosure also provides the above intermediates.

On the other hand, the present disclosure provides a use of the aforementioned compounds and/or intermediates in preparation of a drug or kit, and the drug or kit is used to inhibit expression of an ANGPTL3 gene.

Preferably, the drug or kit is used to prevent and/or treat a dyslipidemia disease; and further preferably, the dyslipidemia disease includes hyperlipidemia and hypertriglyceridemia.

On the other hand, the present disclosure provides a kit. According to an embodiment of the present disclosure, the kit includes the siRNA and/or the siRNA conjugate.

Therefore, the kit according to the embodiment of the present disclosure may be used to inhibit the expression of the ANGPTL3 gene in the cell, thereby the levels of LDL-C, VLDL-C, HDL-C and TG are reduced, as to prevent and/or treat the hyperlipidemia and the hypertriglyceridemia.

On the other hand, the present disclosure provides a method for inhibiting expression of an ANGPTL3 gene in a subject, and the method includes: administering the previously described siRNA and/or the previously described siRNA conjugate to the subject, as to inhibit the expression of the ANGPTL3 gene.

On the other hand, the present disclosure provides a method for inhibiting expression of an ANGPTL3 gene in a cell. According to an embodiment of the present disclosure, the method includes: transfecting the cell with the siRNA and/or the siRNA conjugate, as to inhibit the expression of the ANGPTL3 gene in the cell.

According to the method for inhibiting the expression of the ANGPTL3 gene in the cell in the embodiment of the present disclosure, the siRNA is used to form RISC, and complementary-paired with the mRNA sequence of the target gene (ANGPTL3 gene) to degrade mRNA of the target gene so as to inhibit the expression of the target gene, and then reduce the levels of LDL-C, VLDL-C, HDL-C and TG.

According to an embodiment of the present disclosure, the cell is derived from a mammal.

Optionally, the cell is derived from a human.

Optionally, the cell is a liver cell.

The siRNA provided by the present disclosure is used to form RISC in the human liver cell, and complementary-paired with the mRNA sequence of the ANGPTL3 gene to degrade mRNA of the ANGPTL3 gene so as to inhibit its expression, and then reduce the levels of LDL-C, VLDL-C, HDL-C and TG.

On the other hand, the present disclosure provides a use of the siRNA and/or the siRNA conjugate in preparation of a drug or a kit. According to an embodiment of the present disclosure, the drug or the kit is used to inhibit the expression of the ANGPTL3 gene.

The siRNA provided by the present disclosure is used to prepare the drug or the kit, and the drug or the kit reduces the expression level of the ANGPTL3 gene in the cell by the siRNA therein, thereby the dyslipidemia diseases are prevented and/or treated.

According to an embodiment of the present disclosure, the drug or the kit is used to prevent and/or treat a dyslipidemia disease.

Optionally, the dyslipidemia disease includes the hyperlipidemia and the hypertriglyceridemia.

Optionally, the drug or the kit is used to inhibit the expression of the ANGPTL3 gene in the cell.

On the other hand, the present disclosure provides a method for preventing and/or treating the dyslipidemia disease. According to an embodiment of the present disclosure, the method includes: administering the siRNA and/or the siRNA conjugate to a subject.

According to an embodiment of the present disclosure, the dyslipidemia disease includes the hyperlipidemia and the hypertriglyceridemia.

Additional aspects and advantages of the present disclosure may be partially given in the following descriptions, and some may become apparent from the following descriptions, or may be understood from the practice of the present disclosure.

The present invention has the following beneficial effects.

After the target ligand provided by the present invention forms the conjugate with siRNA, the conjugate may reduce the expression of ANGPTL3 in both cell model and mouse model, and may reduce the expression quantity by more than 50% compared with a control group, it may be maximally reduced by nearly 90%, and has a good clinical application prospect.

### BRIEF DESCRIPTION OF THE DRAWINGS

The above and/or additional aspects and advantages of the present disclosure may become apparent and easily understood from descriptions of embodiments in combination with the following drawings, herein:

FIG. 1 shows an expression result of an ANGPTL3 gene (abbreviated as ANL3 in the figure) in a Hep 3B cell detected by a quantitative real-time PCR after the cell is transfected by some siRNAs in Table 2 at 0.1 nM concentration.

FIG. 2 shows an expression result of the ANGPTL3 gene (abbreviated as ANL3 in the figure) in the Hep 3B cell detected by the quantitative real-time PCR after the cell is transfected by some siRNAs in Table 2 at 10 nM concentration.

FIG. 3 shows a GalNAc-siRNA conjugate synthesized in Embodiment 3, herein the structure of the conjugate in the second column includes three portions: Target-Type of Modification-siRNA. For example, the structure of G1043-S2A2-A265 is: the 1043 target is linked with the 5′-end of the siRNA sense chain numbered as A265 by the thiophosphate bond, and S2A2 is the type of modification to siRNA of A265. The specific modification groups and modification modes are as follows.

In the nucleic acid sequence, Ao represents an adenosine, Uo represents a uridine, Go represents a guanosine, and Co represents a cytosine, and there is no symbol between directly adjacent nucleotides, it is indicated that it is linked by a normal phosphate bond.

DNA: AGCT (A represents 2′-deoxyadenosine, T represents 2′-deoxythymidine, G represents 2′-deoxyguanosine, and C represents 2′-deoxycytidine).

2′-F: aFgFcFuF (aF represents 2′-fluoroadenine nucleoside, uF represents 2′-fluorouracil nucleoside, gF represents 2′-fluoroguanine nucleoside, and cF represents 2′-fluorocytosine nucleoside).

2′-OMe: aMgMcMuM (aM represents 2′-O-methyladenine nucleoside, uM represents 2′-O-methyluracil nucleoside, gM represents 2′-O-methylguanine nucleoside, and cM represents 2′-O-methylcytosine nucleoside).

*: represents that it is linked by a thiophosphate bond.

y and z in the sequence represent the position of the target.

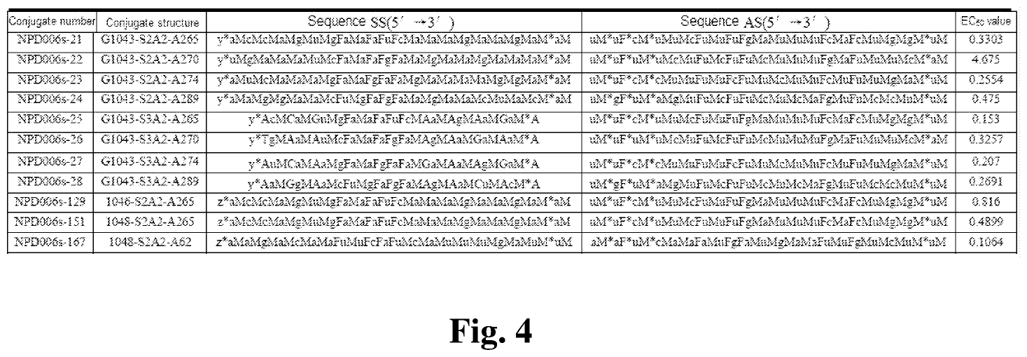

FIG. 4 shows an activity test result (EC₅₀ value) of each conjugate in Embodiment 4.The conjugates in FIG. 4 are included in FIG. 3

### DETAILED DESCRIPTION OF THE EMBODIMENTS

Embodiments of the present disclosure are described in detail below. The embodiments described below are exemplary, and are only used to explain the present disclosure, but may not be understood as limitation to the present disclosure.

“Pharmaceutically acceptable carriers” are recognized in the field, including a pharmaceutically acceptable material, composition or carrier suitable for applying a compound of the present disclosure to a mammal. The carrier includes a liquid or solid filler, a diluent, an excipient, a solvent or an encapsulation material that is involved in carrying or transferring a subject substance from one organ or a part of a body to another organ or another part of the body. Each carrier must be “acceptable” in the sense that it is compatible with other components in a preparation and harmless to a patient. Some examples of materials that may be used as the pharmaceutically acceptable carriers include: sugars, such as a lactose, a glucose, and a sucrose; starches, such as a corn starch and a potato starch; a cellulose and its derivatives, such as a sodium carboxymethyl cellulose, an ethyl cellulose and a cellulose acetate, a powder-like tragacanth gum, a malt, a gelatin, and talcum powder; excipients, such as a cocoa butter and a suppository wax; oils, such as peanut oil, cottonseed oil, safflower oil, sesame oil, olive oil, corn oil and soybean oil; glycols, such as a propylene glycol; polyols, such as a glycerin, a sorbitol, a mannitol and a polyethylene glycol; esters, such as an ethyl oleate and an ethyl laurate; an agar; buffer agents, such as a magnesium hydroxide and an aluminum hydroxide; an alginic acid; pyrogen-free water; Ringer's solution; ethanol; phosphate buffer solution; and other non-toxic compatible substances used in the pharmaceutical preparation.

A wetting agent, an emulsifier and a lubricant such as a sodium dodecyl sulfate and a magnesium stearate, as well as a colorant, a releasing agent, a coating agent, a sweetening agent, a flavoring agent and an aromatic agent, a preservative and an antioxidant may also be present in the composition.

The pharmaceutical composition of the present disclosure includes those suitable for oral, nasal, topical, buccal, sublingual, rectal, and/or parenteral administration. The preparation may conveniently exist in the form of a unit dosage form and may be prepared by any methods well-known in the pharmaceutical field. The amount of an active ingredient that may be combined with the carrier substance to prepare a single dosage form is generally the amount of the compound that produces the therapeutic effect. In general, in the unit of 1%, the amount of the active ingredient is about 1% to about 99%, preferably about 5% to about 70%, and most preferably about 10% to about 30%.

A term “treatment” is used to refer to obtain the desired pharmacological and/or physiological effect. The effect may be preventive in terms of completely or partially preventing a disease or its symptoms, and/or therapeutic in terms of partially or completely curing the disease and/or adverse effects caused by the disease. The “treatment” used herein encompasses diseases of mammals, especially human diseases, including: (a) prevention of diseases or symptoms in individuals who are prone to disease but are not diagnosed with the diseases yet; (b) inhibition of the diseases, such as retardation of disease development; or (c) remission of the diseases, such as the symptoms related to the diseases are alleviated. The “treatment” used herein encompasses any medication that gives a drug or a compound to the individual to treat, cure, remit, improve, alleviate or inhibit the diseases of the individual, including but not limited to giving the drug containing the compound described herein to the individual in need.

The present disclosure provides a siRNA for inhibiting expression of ANGPTL3. According to an embodiment of the present disclosure, the siRNA includes a sense chain and an antisense chain, and the antisense chain includes a complementary region complementary-paired to the sense chain, herein the sense chain is selected from a nucleotide sequence that is not more than 5 nucleotides different from a nucleotide sequence of each chain in SEQ ID NO: 1-SEQ ID NO: 154, and the antisense chain is selected from a nucleotide sequence that is not more than 5 nucleotides different from a nucleotide sequence of each chain in SEQ ID NO: 155-SEQ ID NO: 308.

According to an embodiment of the present disclosure, the sense chain includes not only SEQ ID NO: 1-SEQ ID NO: 154 shown in Table 2, but also a continuous nucleotide sequence that is 1, 2, 3, 4 and 5 nucleotides different from the sense chain shown in Table 2.

According to an embodiment of the present disclosure, the antisense chain includes not only SEQ ID NO: 155-SEQ ID NO: 308 shown in Table 2, but also a continuous nucleotide sequence that is 1, 2, 3, 4 and 5 nucleotides different from the antisense chain shown in Table 2.

According to an embodiment of the present disclosure, the siRNA includes at least one modified nucleotide.

The modified nucleotide is selected from at least one of the following:
a 5′-thiophosphate based nucleotide, a 5-methylcytosine nucleotide, a 2′-O-methyl modified nucleotide, a 2′-O-2-methoxyethyl modified nucleotide, a 2′-fluoro modified nucleotide, a 3′-nitrogen substituted modified nucleotide, a 2′-deoxy-2′-fluoro modified nucleotide, a 2′-deoxy modified nucleotide, a locked nucleotide, a de-base nucleotide, a 2′-amino modified nucleotide, a morpholino nucleotide, a polypeptide nucleotide, an amino phosphate, and a nucleotide including a nonnatural base.

According to an embodiment of the present disclosure, the length of the complementary region is 18-21 bp, for example, 19 bp.

According to an embodiment of the present disclosure, the lengths of the sense chain and the antisense chain in the siRNA are 18-25 bp, for example, 21 bp.

According to a specific embodiment of the present disclosure, the lengths of the sense chain and the antisense chain in the siRNA are 21 bp, and bases in the sense chain and the antisense chain are complementary one by one, or 19 consecutive bases are complementary in the sense chain and the antisense chain in the siRNA, namely the length of the complementary region is 19 bp.

According to an embodiment of the present disclosure, a liver cell is transfected with the siRNA, as to inhibit the expression of the ANGPTL3 gene in the cell.

For an ANGPTL3 gene target, the inventor of the present disclosure designs an appropriate small interfering nucleic acid sequence, synthesizes the siRNA, uses a transfection reagent to introduce the siRNA into the cell, forms RISC, specifically recognizes and targets the mRNA sequence that binds to the target gene, and cuts mRNA between 10-11 bases from a 5′-end, thus the post-transcriptional gene silencing is caused, and the expression of an ANGPTL3 secreted protein is regulated.

According to an embodiment of the present disclosure, the siRNA is linked with a target ligand by a covalent bond.

According to an embodiment of the present disclosure, the target ligand includes at least one N-acetyl-galactosamine.

According to an embodiment of the present disclosure, the target ligand is linked to the sense chain in the siRNA.

The embodiments of the present disclosure are described in detail below. The embodiments described below are exemplary, and are only used to explain the present disclosure, but may not be understood as limitation to the present disclosure. If no specific technologies or conditions are indicated in the embodiments, it is performed according to the technologies or conditions described in documents in this field or product instructions. Reagents or instruments used that do not indicate manufacturers are all conventional products that may be purchased in the market.

Some synthetic routes of this embodiment may refer to CN202110397429.9 and CN202110008013.3; and the embodiments of the present application add the above two patent applications in a mode of source citation.

Embodiment 1: Activity Test of Small Interfering Nucleic Acid (siRNA) by In Vitro Cell Model (Hep 3B Cell) 1) Preparation of suspension transfection reagent: the concentration of siRNA mother liquor is 50 μM. A diethylpyrocarbonate (DEPC) is diluted with water to obtain 10 μM of a siRNA system, 50 μL of Opti-MEM is diluted to obtain 0.2 μM of the siRNA system, it is blown and sucked for 3-5 times and mixed uniformly (the final concentration is 10 nM). 50 μL of Opti-MEM is diluted with 0.50 μL of 0.2 μM siRNA to obtain 0.002 μM of the siRNA system, and it is blown and sucked for 3-5 times and mixed uniformly (the final concentration is 0.1 nM); and 50 μL of Opti-MEM is diluted with 2 μL of RNAiMAX, and it is blown and sucked for 3-5 times and mixed uniformly. A transfection reagent and a small interfering nucleic acid diluent are respectively mixed, it is blown and sucked for 3-5 times and mixed uniformly, and stilly placed for 10 min at a room temperature.

2) Cell treatment: it is observed under a microscope that the convergence rate of a Hep 3B cell line is >70%, cells are spread on a 12-well plate according to 2×10⁵ cells/well, 900 μL of a Dulbecco's Modified Eagle Medium (DMEM) containing 10% fetal bovine serum (FBS) is added per well, and a transfection complex is added to the 12-well plate, and cultured in a 5% CO2 incubator at 37° C.

3) After 24 h, the total RNA of the cells are extracted, and the expression conditions of the ANGPTL3 mRNA sequence in the cell is detected by the quantitative real-time PCR, herein PCR primers used to amplify internal reference genes peptidylprolylisomerase B (PPIB) and ANGPTL3 are shown in Table 1.

TABLE 1
PCR primer sequence for amplification of
internal reference genes PPIB and ANGPTL3

| | SEQ | |
|---|---|---|
| Gene name | ID NO. | Nucleotide sequence (5′-3′) |
| Human PPIB | 309 | GGTGATCTTTGGTCTCTTCGG |
| | 310 | TAGATGCTCTTTCCTCCTGTG |
| Human ANGPTL3 | 311 | ATTTTAGCCAATGGCCTCCTTC |
| | 312 | CTGGTTTGCAGCGATAGATCATA |

4) The inhibition rate of the small interfering nucleic acid on the expression level of ANGPTL3 is calculated according to the following formula: inhibition rate=[1−(expression quantity of ANGPTL3 mRNA in experimental group/expression quantity of PPIB mRNA in experimental group)/(expression quantity of ANGPTL3 mRNA in negative control group/expression quantity of PPIB mRNA in negative control group)]×100%. Herein, each experimental group is the cells treated with the small interfering nucleic acid respectively; and the negative control group (marked as Blank) is the cells without any small interfering nucleic acid treatment.

The above method is used to obtain the results of the inhibition rate of the ANGPTL3 gene (NM_014495.4) expression after the Hep 3B cell is transfected by 154 pairs of siRNAs in Table 2 at the concentrations of 0.1 nM and 10 nM respectively.

TABLE 2
154 pairs of siRNA sequences targeting ANGPTL3

| | | Sense chain | SEQ | Antisense chain | SEQ | Inhibition rate (%) | |
|---|---|---|---|---|---|---|---|
| Name | Position | (5′-3′) | ID NO. | (5′-3′) | ID NO. | 0.1 nM | 10 nM |
| A129 | 98-118 | CAGAAUUGAUCA | 1 | AUUGUCUUGAUCA | 155 | 88 | 78 |
| | | AGACAAUUC | | AUUCUGGA | | | |
| A554 | 523-543 | CAGAAGUAACUU | 2 | UUAAGUGAAGUUA | 156 | 78 | 70 |
| | | CACUUAAAA | | CUUCUGGG | | | |
| A566 | 535-555 | CACUUAAAACUU | 3 | UCUACAAAAGUUU | 157 | 44 | 70 |
| | | UUGUAGAAA | | UAAGUGAA | | | |
| A568 | 537-557 | CUUAAAACUUUU | 4 | UUUCUACAAAAGU | 158 | 58 | 56 |
| | | GUAGAAAAA | | UUUAAGUG | | | |
| A749 | 718-738 | GAACUACUCCCU | 5 | UGAAGAAAGGGAG | 159 | 82 | 85 |
| | | UUCUUCAGU | | UAGUUCUU | | | |
| A889 | 858-878 | CAUGUCUACUGU | 6 | UAACAUCACAGUA | 160 | 62 | 78 |
| | | GAUGUUAUA | | GACAUGAA | | | |
| A1053 | 1022-1042 | GCAAUCUAAUUA | 7 | UAAAACAUAAUUA | 161 | 41 | 79 |
| | | UGUUUUACG | | GAUUGCUU | | | |
| A1058 | 1027-1047 | CUAAUUAUGUUU | 8 | AUUCGUAAAACAU | 162 | 52 | 88 |
| | | UACGAAUUG | | AAUUAGAU | | | |
| A1145 | 1114-1134 | CCAACUAUACGC | 9 | AGAUGUAGCGUAU | 163 | 72 | 90 |
| | | UACAUCUAG | | AGUUGGUU | | | |
| A13 | 60-80 | AAGCUCCUUCUU | 10 | AACAAUAAAAAGA | 164 | 84 | 93 |
| | | UUUAUUGUU | | AGGAGCUU | | | |
| A32 | 79-99 | UUCCUCUAGUUA | 11 | UGGAGGAAAUAAC | 165 | 64 | 64 |
| | | UUUCCUCCA | | UAGAGGAA | | | |
| A35 | 82-102 | CUCUAGUUAUUU | 12 | UUCUGGAGGAAAU | 166 | 86 | 90 |
| | | CCUCCAGAA | | AACUAGAG | | | |
| A45 | 92-112 | UUCCUCCAGAAU | 13 | UCUUGAUCAAUUC | 167 | 82 | 87 |
| | | UGAUCAAGA | | UGGAGGAA | | | |
| A48 | 95-115 | CUCCAGAAUUGA | 14 | UUGUCUUGAUCAA | 168 | 84 | 90 |
| | | UCAAGACAA | | UUCUGGAG | | | |
| A49 | 96-116 | UCCAGAAUUGAU | 15 | AUUGUCUUGAUCA | 169 | 79 | 89 |
| | | CAAGACAAU | | AUUCUGGA | | | |
| A56 | 103-123 | UUGAUCAAGACA | 16 | AUGAUGAAUUGUC | 170 | 78 | 88 |
| | | AUUCAUCAU | | UUGAUCAA | | | |
| A62 | 109-129 | AAGACAAUUCAU | 17 | AAUCAAAUGAUGA | 171 | 77 | 88 |
| | | CAUUUGAUU | | AUUGUCUU | | | |
| A64 | 111-131 | GACAAUUCAUCA | 18 | AGAAUCAAAUGAU | 172 | 69 | 84 |
| | | UUUGAUUCU | | GAAUUGUC | | | |
| A118 | 165-185 | UUAGACGAUGUA | 19 | UAAAAUUUUUACA | 173 | 79 | 79 |
| | | AAAAUUUUA | | UCGUCUAA | | | |
| A182 | 229-249 | UCCAUAAGACGA | 20 | UUUGGCCCUUCGU | 174 | 23 | 50 |
| | | AGGGCCAAA | | CUUAUGGA | | | |
| A189 | 236-256 | GACGAAGGGCCA | 21 | UCAUUAAUUUGGC | 175 | 55 | 57 |
| | | AAUUAAUGA | | CCUUCGUC | | | |
| A206 | 253-273 | AUGACAUAUUUC | 22 | UGAGUUUUUGAAA | 176 | 69 | 82 |
| | | AAAAACUCA | | UAUGUCAU | | | |
| A207 | 254-274 | UGACAUAUUUCA | 23 | UUGAGUUUUUGAA | 177 | 75 | 93 |
| | | AAAACUCAA | | AUAUGUCA | | | |
| A1209 | 1256-1276 | GUGGCAUGAUGA | 24 | UCUCCACACUCAUC | 178 | 60 | 87 |
| | | GUGUGGAGA | | AUGCCAC | | | |
| A236 | 283-303 | AUCAGUCUUUUU | 25 | AUAGAUCAUAAAA | 179 | 48 | 75 |
| | | AUGAUCUAU | | AGACUGAU | | | |
| A257 | 304-324 | CGCUGCAAACCA | 26 | UGAUUUCACUGGU | 180 | 78 | 87 |
| | | GUGAAAUCA | | UUGCAGCG | | | |
| A259 | 306-326 | CUGCAAACCAGU | 27 | UUUGAUUUCACUG | 181 | 84 | 89 |
| | | GAAAUCAAA | | GUUUGCAG | | | |
| A264 | 311-331 | AACCAGUGAAAU | 28 | UCUUCUUUGAUUU | 182 | 80 | 85 |
| | | CAAAGAAGA | | CACUGGUU | | | |
| A265 | 312-332 | ACCAGUGAAAUC | 29 | UUCUUCUUUGAUU | 183 | 66 | 80 |
| | | AAAGAAGAA | | UCACUGGU | | | |
| A267 | 314-334 | CAGUGAAAUCAA | 30 | UCUUCUUCUUUGA | 184 | 62 | 76 |
| | | AGAAGAAGA | | UUUCACUG | | | |
| A270 | 317-337 | UGAAAUCAAAGA | 31 | UUUUCUUCUUCUU | 185 | 48 | 86 |
| | | AGAAGAAAA | | UGAUUUCA | | | |
| A274 | 321-341 | AUCAAAGAAGAA | 32 | UUCCUUUUCUUCU | 186 | 50 | 85 |
| | | GAAAAGGAA | | UCUUUGAU | | | |
| A281 | 328-348 | AAGAAGAAAAGG | 33 | UUCUCAGUUCCUU | 187 | 59 | 84 |
| | | AACUGAGAA | | UUCUUCUU | | | |
| A284 | 331-351 | AAGAAAAGGAAC | 34 | UUCUUCUCAGUUC | 188 | 47 | 53 |
| | | UGAGAAGAA | | CUUUUCUU | | | |
| A289 | 336-356 | AAGGAACUGAGA | 35 | UGUAGUUCUUCUC | 189 | 77 | 89 |
| | | AGAACUACA | | AGUUCCUU | | | |
| A290 | 337-357 | AGGAACUGAGAA | 36 | AUGUAGUUCUUCU | 190 | 60 | 87 |
| | | GAACUACAU | | CAGUUCCU | | | |
| A293 | 340-360 | AACUGAGAAGAA | 37 | UAUAUGUAGUUCU | 191 | 70 | 80 |
| | | CUACAUAUA | | UCUCAGUU | | | |
| A294 | 341-361 | ACUGAGAAGAAC | 38 | UUAUAUGUAGUUC | 192 | 43 | 77 |
| | | UACAUAUAA | | UUCUCAGU | | | |
| A319 | 366-386 | CAAGUCAAAAAU | 39 | UACCUCUUCAUUU | 193 | 55 | 82 |
| | | GAAGAGGUA | | UUGACUUG | | | |
| A329 | 376-396 | AUGAAGAGGUAA | 40 | ACAUAUUCUUUAC | 194 | 42 | 64 |
| | | AGAAUAUGU | | CUCUUCAU | | | |
| A346 | 393-413 | AUGUCACUUGAA | 41 | UGAGUUGAGUUCA | 195 | 75 | 79 |
| | | CUCAACUCA | | AGUGACAU | | | |
| A379 | 426-446 | CUCCUAGAAGAA | 42 | UAGAAUUUUUUCU | 196 | 72 | 77 |
| | | AAAAUUCUA | | UCUAGGAG | | | |
| A385 | 432-452 | GAAGAAAAAAUU | 43 | UUGAAGUAGAAUU | 197 | 63 | 80 |
| | | CUACUUCAA | | UUUUCUUC | | | |
| A390 | 437-457 | AAAAAUUCUACU | 44 | UUUUGUUGAAGUA | 198 | 73 | 81 |
| | | UCAACAAAA | | GAAUUUUU | | | |
| A391 | 438-458 | AAAAUUCUACUU | 45 | UUUUUGUUGAAGU | 199 | 69 | 73 |
| | | CAACAAAAA | | AGAAUUUU | | | |
| A397 | 444-464 | CUACUUCAACAA | 46 | UUUCACUUUUUGU | 200 | 52 | 67 |
| | | AAAGUGAAA | | UGAAGUAG | | | |
| A398 | 445-465 | UACUUCAACAAA | 47 | AUUUCACUUUUUG | 201 | 22 | 28 |
| | | AAGUGAAAU | | UUGAAGUA | | | |
| A401 | 448-468 | UUCAACAAAAAG | 48 | AAUAUUUCACUUU | 202 | 56 | 66 |
| | | UGAAAUAUU | | UUGUUGAA | | | |
| A403 | 450-470 | CAACAAAAAGUG | 49 | UAAAUAUUUCACU | 203 | 47 | 64 |
| | | AAAUAUUUA | | UUUUGUUG | | | |
| A462 | 509-529 | AACUCCAGAACA | 50 | ACUUCUGGGUGUU | 204 | 64 | 74 |
| | | CCCAGAAGU | | CUGGAGUU | | | |
| A464 | 511-531 | CUCCAGAACACC | 51 | UUACUUCUGGGUG | 205 | 51 | 81 |
| | | CAGAAGUAA | | UUCUGGAG | | | |
| A473 | 520-540 | ACCCAGAAGUAA | 52 | UAAGUGAAGUUAC | 206 | 56 | 71 |
| | | CUUCACUUA | | UUCUGGGU | | | |
| A475 | 522-542 | CCAGAAGUAACU | 53 | UUUAAGUGAAGUU | 207 | 65 | 68 |
| | | UCACUUAAA | | ACUUCUGG | | | |
| A476 | 523-543 | CAGAAGUAACUU | 54 | UUUUAAGUGAAGU | 208 | 65 | 71 |
| | | CACUUAAAA | | UACUUCUG | | | |
| A479 | 526-546 | AAGUAACUUCAC | 55 | AAGUUUUAAGUGA | 209 | 21 | 78 |
| | | UUAAAACUU | | AGUUACUU | | | |
| A483 | 530-550 | AACUUCACUUAA | 56 | ACAAAAGUUUUAA | 210 | 39 | 34 |
| | | AACUUUUGU | | GUGAAGUU | | | |
| A495 | 542-562 | AACUUUUGUAGA | 57 | UCUUGUUUUUCUA | 211 | 55 | 76 |
| | | AAAACAAGA | | CAAAAGUU | | | |
| A500 | 547-567 | UUGUAGAAAAAC | 58 | UAUUAUCUUGUUU | 212 | 52 | 54 |
| | | AAGAUAAUA | | UUCUACAA | | | |
| A508 | 555-575 | AAACAAGAUAAU | 59 | UUUGAUGCUAUUA | 213 | 50 | 74 |
| | | AGCAUCAAA | | UCUUGUUU | | | |
| A519 | 566-586 | UAGCAUCAAAGA | 60 | UGGAGAAGGUCUU | 214 | 69 | 77 |
| | | CCUUCUCCA | | UGAUGCUA | | | |
| A537 | 584-604 | CCAGACCGUGGA | 61 | UAUUGGUCUUCCA | 215 | 64 | 25 |
| | | AGACCAAUA | | CGGUCUGG | | | |
| A538 | 585-605 | CAGACCGUGGAA | 62 | AUAUUGGUCUUCC | 216 | 43 | — |
| | | GACCAAUAU | | ACGGUCUG | | | |
| A540 | 587-607 | GACCGUGGAAGA | 63 | UUAUAUUGGUCUU | 217 | 37 | 58 |
| | | CCAAUAUAA | | CCACGGUC | | | |
| A541 | 588-608 | ACCGUGGAAGAC | 64 | UUUAUAUUGGUCU | 218 | 40 | 59 |
| | | CAAUAUAAA | | UCCACGGU | | | |
| A544 | 591-611 | GUGGAAGACCAA | 65 | UUGUUUAUAUUGG | 219 | Invalid | 38 |
| | | UAUAAACAA | | UCUUCCAC | | | |
| A547 | 594-614 | GAAGACCAAUAU | 66 | UAAUUGUUUAUAU | 220 | 36 | 60 |
| | | AAACAAUUA | | UGGUCUUC | | | |
| A548 | 595-615 | AAGACCAAUAUA | 67 | UUAAUUGUUUAUA | 221 | 11 | 13 |
| | | AACAAUUAA | | UUGGUCUU | | | |
| A568 | 615-635 | AACCAACAGCAU | 68 | UAUUUGACUAUGC | 222 | 24 | 58 |
| | | AGUCAAAUA | | UGUUGGUU | | | |
| A569 | 616-636 | ACCAACAGCAUA | 69 | UUAUUUGACUAUG | 223 | 17 | 36 |
| | | GUCAAAUAA | | CUGUUGGU | | | |
| A579 | 626-646 | UAGUCAAAUAAA | 70 | UCUAUUUCUUUUA | 224 | 29 | 72 |
| | | AGAAAUAGA | | UUUGACUA | | | |
| A582 | 629-649 | UCAAAUAAAAGA | 71 | UUUUCUAUUUCUU | 225 | 22 | 44 |
| | | AAUAGAAAA | | UUAUUUGA | | | |
| A602 | 649-669 | AUCAGCUCAGAA | 72 | UACUAGUCCUUCU | 226 | 47 | 75 |
| | | GGACUAGUA | | GAGCUGAU | | | |
| A604 | 651-671 | CAGCUCAGAAGG | 73 | AAUACUAGUCCUU | 227 | 44 | 69 |
| | | ACUAGUAUU | | CUGAGCUG | | | |
| A607 | 654-674 | CUCAGAAGGACU | 74 | UUGAAUACUAGUC | 228 | 36 | 67 |
| | | AGUAUUCAA | | CUUCUGAG | | | |
| A609 | 656-676 | CAGAAGGACUAG | 75 | UCUUGAAUACUAG | 229 | 21 | 49 |
| | | UAUUCAAGA | | UCCUUCUG | | | |
| A618 | 665-685 | UAGUAUUCAAGA | 76 | UCUGUGGGUUCUU | 230 | 16 | 61 |
| | | ACCCACAGA | | GAAUACUA | | | |
| A629 | 676-696 | AACCCACAGAAA | 77 | AUAGAGAAAUUUC | 231 | 29 | 38 |
| | | UUUCUCUAU | | UGUGGGUU | | | |
| A652 | 699-719 | UCCAAGCCAAGA | 78 | UCUUGGUGCUCUU | 232 | 40 | 78 |
| | | GCACCAAGA | | GGCUUGGA | | | |
| A655 | 702-722 | AAGCCAAGAGCA | 79 | AGUUCUUGGUGCU | 233 | 44 | 70 |
| | | CCAAGAACU | | CUUGGCUU | | | |
| A675 | 722-745 | UACUCCCUUUCU | 80 | UUCAACUGAAGAA | 234 | 50 | 72 |
| | | UCAGUUGAA | | AGGGAGUA | | | |
| A678 | 725-745 | UCCCUUUCUUCA | 81 | UCAUUCAACUGAA | 235 | 57 | 73 |
| | | GUUGAAUGA | | GAAAGGGA | | | |
| A686 | 733-753 | UUCAGUUGAAUG | 82 | UUCUUAUUUCAUU | 236 | 36 | 55 |
| | | AAAUAAGAA | | CAACUGAA | | | |
| A687 | 734-754 | UCAGUUGAAUGA | 83 | UUUCUUAUUUCAU | 237 | 34 | 74 |
| | | AAUAAGAAA | | UCAACUGA | | | |
| A691 | 738-758 | UUGAAUGAAAUA | 84 | UACAUUUCUUAUU | 238 | 52 | 72 |
| | | AGAAAUGUA | | UCAUUCAA | | | |
| A725 | 772-792 | UUCCUGCUGAAU | 85 | UGGUGGUACAUUC | 239 | 21 | 60 |
| | | GUACCACCA | | AGCAGGAA | | | |
| A729 | 776-796 | UGCUGAAUGUAC | 86 | UAAAUGGUGGUAC | 240 | 22 | 51 |
| | | CACCAUUUA | | AUUCAGCA | | | |
| A731 | 778-798 | CUGAAUGUACCA | 87 | UAUAAAUGGUGGU | 241 | 58 | 77 |
| | | CCAUUUAUA | | ACAUUCAG | | | |
| A739 | 786-806 | ACCACCAUUUAU | 88 | ACCUCUGUUAUAA | 242 | 22 | 35 |
| | | AACAGAGGU | | AUGGUGGU | | | |
| A741 | 788-808 | CACCAUUUAUAA | 89 | UCACCUCUGUUAU | 243 | 46 | 74 |
| | | CAGAGGUGA | | AAAUGGUG | | | |
| A742 | 789-809 | ACCAUUUAUAAC | 90 | UUCACCUCUGUUA | 244 | 23 | 72 |
| | | AGAGGUGAA | | UAAAUGGU | | | |
| A751 | 798-818 | AACAGAGGUGAA | 91 | ACUUGUAUGUUCA | 245 | 21 | 68 |
| | | CAUACAAGU | | CCUCUGUU | | | |
| A755 | 802-822 | GAGGUGAACAUA | 92 | UGCCACUUGUAUG | 246 | Invalid | 59 |
| | | CAAGUGGCA | | UUCACCUC | | | |
| A758 | 805-825 | GUGAACAUACAA | 93 | ACAUGCCACUUGU | 247 | 15 | 55 |
| | | GUGGCAUGU | | AUGUUCAC | | | |
| A765 | 812-832 | UACAAGUGGCAU | 94 | AUGGCAUACAUGC | 248 | 2 | Invalid |
| | | GUAUGCCAU | | CACUUGUA | | | |
| A798 | 845-865 | CUCUCAAGUUUU | 95 | UAGACAUGAAAAA | 249 | 40 | 64 |
| | | UCAUGUCUA | | CUUGAGAG | | | |
| A809 | 856-876 | UUCAUGUCUACU | 96 | UAACAUCACAGUA | 250 | 66 | 69 |
| | | GUGAUGUUA | | GACAUGAA | | | |
| A811 | 858-878 | CAUGUCUACUGU | 97 | UAUAACAUCACAG | 251 | 30 | 54 |
| | | GAUGUUAUA | | UAGACAUG | | | |
| A814 | 861-881 | GUCUACUGUGAU | 98 | UGAUAUAACAUCA | 252 | 70 | 74 |
| | | GUUAUAUCA | | CAGUAGAC | | | |
| A817 | 864-884 | UACUGUGAUGUU | 99 | ACCUGAUAUAACA | 253 | 65 | 63 |
| | | AUAUCAGGU | | UCACAGUA | | | |
| A833 | 880-900 | CAGGUAGUCCAU | 100 | UUAAUGUCCAUGG | 254 | 34 | 36 |
| | | GGACAUUAA | | ACUACCUG | | | |
| A854 | 901-921 | UUCAACAUCGAA | 101 | AUCCAUCUAUUCG | 255 | 27 | 44 |
| | | UAGAUGGAU | | AUGUUGAA | | | |
| A866 | 913-933 | UAGAUGGAUCAC | 102 | UGAAGUUUUGUGA | 256 | 71 | 83 |
| | | AAAACUUCA | | UCCAUCUA | | | |
| A870 | 917-937 | UGGAUCACAAAA | 103 | UCAUUGAAGUUUU | 257 | 75 | 79 |
| | | CUUCAAUGA | | GUGAUCCA | | | |
| A875 | 922-942 | CACAAAACUUCA | 104 | ACGUUUCAUUGAA | 258 | 68 | 78 |
| | | AUGAAACGU | | GUUUUGUG | | | |
| A887 | 934-954 | AUGAAACGUGGG | 105 | UGUAGUUCUCCCA | 259 | 74 | 76 |
| | | AGAACUACA | | CGUUUCAU | | | |
| A891 | 938-958 | AACGUGGGAGAA | 106 | UAUUUGUAGUUCU | 260 | 36 | 48 |
| | | CUACAAAUA | | CCCACGUU | | | |
| A898 | 945-965 | GAGAACUACAAA | 107 | AAAACCAUAUUUG | 261 | 63 | 73 |
| | | UAUGGUUUU | | UAGUUCUC | | | |
| A1004 | 1051-1071 | UGGAAGACUGGA | 108 | UGUUGUCUUUCCA | 262 | 43 | 76 |
| | | AAGACAACA | | GUCUUCCA | | | |
| A1006 | 1053-1073 | GAAGACUGGAAA | 109 | UUUGUUGUCUUUC | 263 | 70 | 79 |
| | | GACAACAAA | | CAGUCUUC | | | |
| A1011 | 1058-1078 | CUGGAAAGACAA | 110 | UAAUGUUUGUUGU | 264 | 72 | 77 |
| | | CAAACAUUA | | CUUUCCAG | | | |
| A1012 | 1059-1079 | UGGAAAGACAAC | 111 | AUAAUGUUUGUUG | 265 | 60 | 74 |
| | | AAACAUUAU | | UCUUUCCA | | | |
| A1018 | 1065-1085 | GACAACAAACAU | 112 | UUCAAUAUAAUGU | 266 | 57 | 85 |
| | | UAUAUUGAA | | UUGUUGUC | | | |
| A1021 | 1068-1088 | AACAAACAUUAU | 113 | AUAUUCAAUAUAA | 267 | 41 | 25 |
| | | AUUGAAUAU | | UGUUUGUU | | | |
| A1025 | 1072-1092 | AACAUUAUAUUG | 114 | AAGAAUAUUCAAU | 268 | 38 | 62 |
| | | AAUAUUCUU | | AUAAUGUU | | | |
| A1066 | 1113-1133 | ACCAACUAUACG | 115 | UAGAUGUAGCGUA | 269 | 75 | 84 |
| | | CUACAUCUA | | UAGUUGGU | | | |
| A1074 | 1121-1141 | UACGCUACAUCU | 116 | AUCGCAACUAGAU | 270 | 69 | 83 |
| | | AGUUGCGAU | | GUAGCGUA | | | |
| A1075 | 1122-1142 | ACGCUACAUCUA | 117 | AAUCGCAACUAGA | 271 | 72 | 80 |
| | | GUUGCGAUU | | UGUAGCGU | | | |
| A1082 | 1129-1149 | AUCUAGUUGCGA | 118 | UGCCAGUAAUCGC | 272 | 45 | 25 |
| | | UUACUGGCA | | AACUAGAU | | | |
| A1097 | 1144-1164 | CUGGCAAUGUCC | 119 | UUGCAUUGGGGAC | 273 | 59 | 62 |
| | | CCAAUGCAA | | AUUGCCAG | | | |
| A1106 | 1153-1173 | UCCCCAAUGCAA | 120 | UUUCCGGGAUUGC | 274 | Invalid | 13 |
| | | UCCCGGAAA | | AUUGGGGA | | | |
| A1107 | 1154-1174 | CCCCAAUGCAAU | 121 | UUUUCCGGGAUUG | 275 | 45 | 54 |
| | | CCCGGAAAA | | CAUUGGGG | | | |
| A1119 | 1166-1186 | CCCGGAAAACAA | 122 | ACCAAAUCUUUGU | 276 | 6 | 43 |
| | | AGAUUUGGU | | UUUCCGGG | | | |
| A1158 | 1205-1225 | CAAAGCAAAAGG | 123 | UUGAAGUGUCCUU | 277 | 23 | 73 |
| | | ACACUUCAA | | UUGCUUUG | | | |
| A1160 | 1207-1227 | AAGCAAAAGGAC | 124 | AGUUGAAGUGUCC | 278 | 46 | 76 |
| | | ACUUCAACU | | UUUUGCUU | | | |
| A1167 | 1214-1234 | AGGACACUUCAA | 125 | UCUGGACAGUUGA | 279 | 26 | 48 |
| | | CUGUCCAGA | | AGUGUCCU | | | |
| A1171 | 1218-1238 | CACUUCAACUGU | 126 | ACCCUCUGGACAG | 280 | 48 | 74 |
| | | CCAGAGGGU | | UUGAAGUG | | | |
| A1174 | 1221-1241 | UUCAACUGUCCA | 127 | AUAACCCUCUGGA | 281 | 54 | 79 |
| | | GAGGGUUAU | | CAGUUGAA | | | |
| A1184 | 1231-1251 | CAGAGGGUUAUU | 128 | AGCCUCCUGAAUA | 282 | 33 | 27 |
| | | CAGGAGGCU | | ACCCUCUG | | | |
| A1204 | 1251-1271 | UGGUGGUGGCAU | 129 | ACACUCAUCAUGC | 283 | 39 | 58 |
| | | GAUGAGUGU | | CACCACCA | | | |
| A1207 | 1254-1274 | UGGUGGCAUGAU | 130 | UCCACACUCAUCA | 284 | 44 | 74 |
| | | GAGUGUGGA | | UGCCACCA | | | |
| A1210 | 1257-1277 | UGGCAUGAUGAG | 131 | UUCUCCACACUCA | 285 | 52 | 78 |
| | | UGUGGAGAA | | UCAUGCCA | | | |
| A1211 | 1258-1278 | GGCAUGAUGAGU | 132 | UUUCUCCACACUC | 286 | 44 | 74 |
| | | GUGGAGAAA | | AUCAUGCC | | | |
| A1241 | 1288-1308 | AUGGUAAAUAUA | 133 | UUGGUUUGUUAUA | 287 | 32 | 81 |
| | | ACAAACCAA | | UUUACCAU | | | |
| A1253 | 1300-1320 | ACAAACCAAGAG | 134 | UAGAUUUUGCUCU | 288 | 74 | 82 |
| | | CAAAAUCUA | | UGGUUUGU | | | |
| A1254 | 1301-1321 | CAAACCAAGAGC | 135 | UUAGAUUUUGCUC | 289 | 53 | 84 |
| | | AAAAUCUAA | | UUGGUUUG | | | |
| A1274 | 1321-1341 | AGCCAGAGAGGA | 136 | AUCCUCUUCUCCUC | 290 | 39 | 70 |
| | | GAAGAGGAU | | UCUGGCU | | | |
| A1276 | 1323-1343 | CCAGAGAGGAGA | 137 | UAAUCCUCUUCUC | 291 | 52 | 68 |
| | | AGAGGAUUA | | CUCUCUGG | | | |
| A1277 | 1324-1344 | CAGAGAGGAGAA | 138 | AUAAUCCUCUUCU | 292 | 50 | 73 |
| | | GAGGAUUAU | | CCUCUCUG | | | |
| A1279 | 1326-1346 | GAGAGGAGAAGA | 139 | AGAUAAUCCUCUU | 293 | 67 | 68 |
| | | GGAUUAUCU | | CUCCUCUC | | | |
| A1284 | 1331-1351 | GAGAAGAGGAUU | 140 | UUCCAAGAUAAUC | 294 | 75 | 35 |
| | | AUCUUGGAA | | CUCUUCUC | | | |
| A1303 | 1350-1370 | AAGUCUCAAAAU | 141 | UAACCUUCCAUUU | 295 | 64 | 74 |
| | | GGAAGGUUA | | UGAGACUU | | | |
| A1305 | 1352-1372 | GUCUCAAAAUGG | 142 | UAUAACCUUCCAU | 296 | 51 | 75 |
| | | AAGGUUAUA | | UUUGAGAC | | | |
| A1310 | 1357-1377 | AAAAUGGAAGGU | 143 | UAGAGUAUAACCU | 297 | 61 | 71 |
| | | UAUACUCUA | | UCCAUUUU | | | |
| A1313 | 1360-1380 | AUGGAAGGUUAU | 144 | UUAUAGAGUAUAA | 298 | 33 | 71 |
| | | ACUCUAUAA | | CCUUCCAU | | | |
| A1314 | 1361-1381 | UGGAAGGUUAUA | 145 | UUUAUAGAGUAUA | 299 | 58 | 79 |
| | | CUCUAUAAA | | ACCUUCCA | | | |
| A1318 | 1365-1385 | AGGUUAUACUCU | 146 | UGAUUUUAUAGAG | 300 | 48 | 71 |
| | | AUAAAAUCA | | UAUAACCU | | | |
| A1345 | 1392-1412 | AUGUUGAUCCAU | 147 | AUCUGUUGGAUGG | 301 | 63 | 80 |
| | | CCAACAGAU | | AUCAACAU | | | |
| A1348 | 1395-1415 | UUGAUCCAUCCA | 148 | UGAAUCUGUUGGA | 302 | 58 | 83 |
| | | ACAGAUUCA | | UGGAUCAA | | | |
| A1351 | 1398-1418 | AUCCAUCCAACA | 149 | UUCUGAAUCUGUU | 303 | 61 | 72 |
| | | GAUUCAGAA | | GGAUGGAU | | | |
| A1352 | 1399-1419 | UCCAUCCAACAG | 150 | UUUCUGAAUCUGU | 304 | 60 | 84 |
| | | AUUCAGAAA | | UGGAUGGA | | | |
| A1355 | 1402-1422 | AUCCAACAGAUU | 151 | AGCUUUCUGAAUC | 305 | 73 | 82 |
| | | CAGAAAGCU | | UGUUGGAU | | | |
| A1356 | 1403-1423 | UCCAACAGAUUC | 152 | AAGCUUUCUGAAU | 306 | 53 | 79 |
| | | AGAAAGCUU | | CUGUUGGA | | | |
| A1359 | 1406-1426 | AACAGAUUCAGA | 153 | UCAAAGCUUUCUG | 307 | 62 | 79 |
| | | AAGCUUUGA | | AAUCUGUU | | | |
| A1361 | 1408-1428 | CAGAUUCAGAAA | 154 | AUUCAAAGCUUUC | 308 | 77 | 88 |
| | | GCUUUGAAU | | UGAAUCUG | | | |

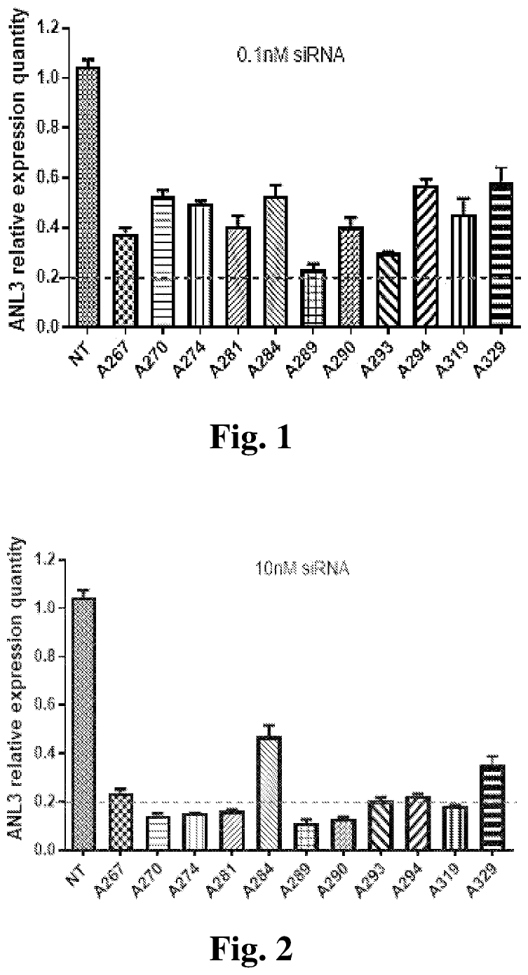

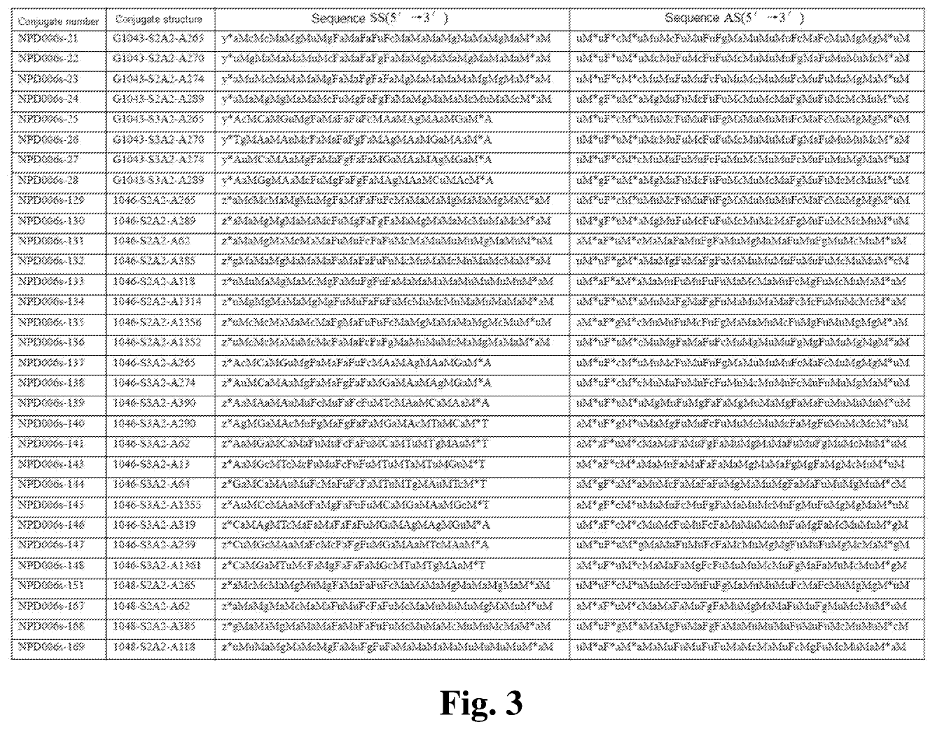

FIGS. 1 and 2 respectively show the results of the expression quantity of the ANGPTL3 gene in the Hep3B cell detected by the quantitative real-time PCR after the cell is transfected by some siRNAs in Table 2 at the concentration of 0.1 nM or 10 nM. It is indicated that the siRNA shown in the drawings may significantly reduce the expression of the ANGPTL3 gene whether the Hep 3B cell is transfected by the siRNA at the 0.1 nM or 10 nM concentration.

Embodiment 2: Synthesis of GalNAc Linkage Target

I. Synthesis of GalNAc Target 1043

According to the following method, a diastereoisomer of TO-23 and TP-23 (a precursor of a 1043 target linked to siRNA) is synthesized.

1. Synthesis of Intermediate GN-17-01

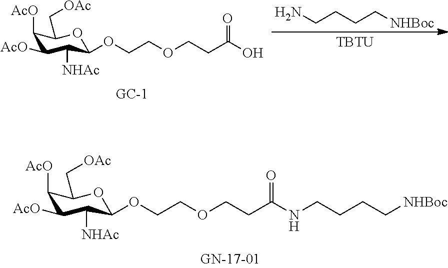

(1) Under an N₂ atmosphere, GC-1 (12 g, 25.89 mmol) is dissolved in a dichloromethane (DCM) (200 mL), the temperature is reduced to 0-5° C. in an ice-water bath, O-benzotriazole-N,N,N′,N′-tetramethyl-uronium-hexafluorophosphate (HBTU) (11.78 g, 31 mmol) and diisopropylethylamine (DIEA) (10 g, 77.67 mmol) are added, and stirred for 10 minutes.

(2) Then, N-tert-butyloxycarbonyl-1,4-butanediamine (4.87 g, 25.89 mmol) is added, the temperature is risen to 25° C. and it is stirred and reacted for 16 hours. A thin-layer chromatography (TLC) shows that raw materials are basically disappeared.

(3) Saturated ammonium chloride solution (100 mL) is added for quenching, solution is separated, and it is extracted by DCM (100 mL×2).

(4) Organic phases are combined and washed with saturated salt water (100 mL), dried with anhydrous Na₂SO₄, filtered and concentrated. After column chromatography purification (DCM/MeOH=20/1), a white solid compound GN-17-01 (15 g, yield: 91%) is obtained.

2. Synthesis of Intermediate GN-17

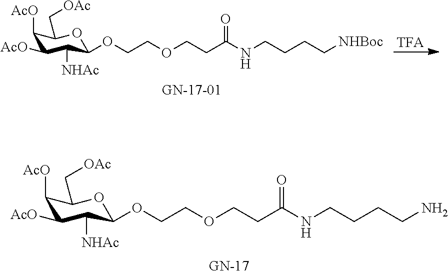

(1) GN-17-01 (15 g, 23.67 mmol) is dissolved in DCM (150 mL), a trifluoroacetic acid (TFA) (50 mL) is added, and stirred at 25° C. for 1 hour. TLC shows that raw materials are basically disappeared and concentrated.

(2) The excess TFA is removed by an acetonitrile (100 mL×3) azeotropic with TFA, to obtain a foam-like solid GN-17 (TFA salt, 12.6 g).

3. Synthesis of Intermediate TO-23-01

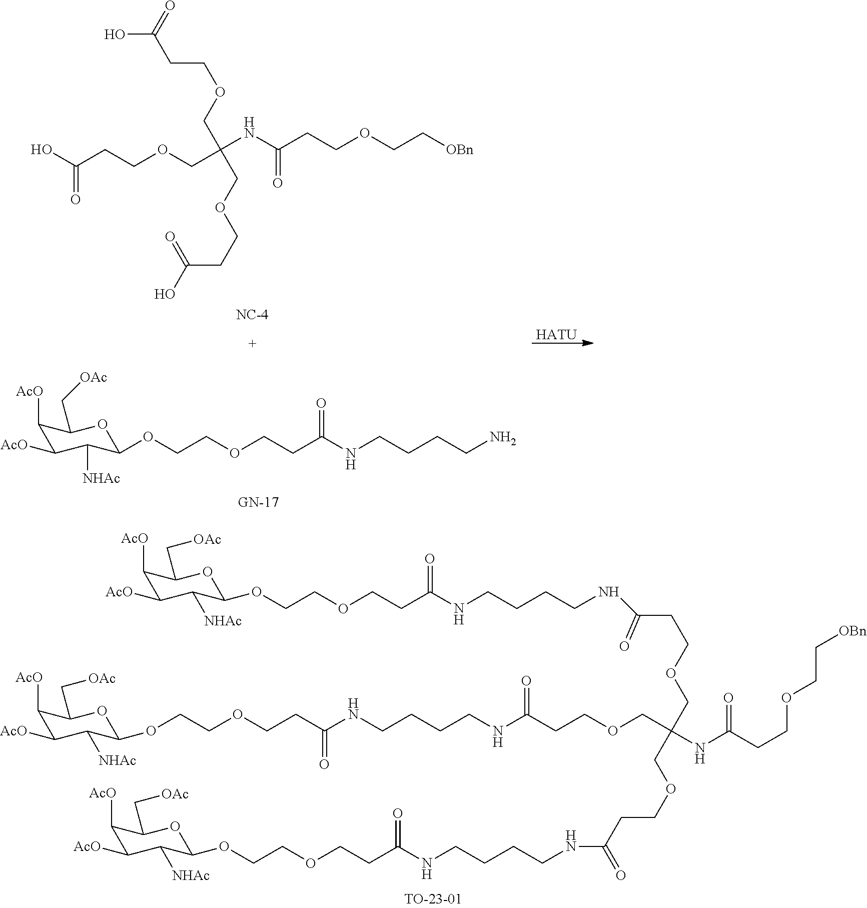 [SMILES: O=C(O)CCOCC(COCCC(=O)O)(COCCC(=O)O)NC(=O)CCOCCOCc1ccccc1 ; "NC-4" ; "HATU" ; CC(=O)NC1C(OCCOCCC(=O)NCCCCN)OC(COC(C)=O)C(OC(C)=O)C1OC(C)=O ; CC(=O)NC1C(OCCOCCC(=O)NCCCCNC(=O)CCOCC(COCCC(=O)NCCCCNC(=O)CCOCCOC2OC(COC(C)=O)C(OC(C)=O)C(OC(C)=O)C2NC(C)=O)(COCCC(=O)NCCCCNC(=O)CCOCCOC2OC(COC(C)=O)C(OC(C)=O)C(OC(C)=O)C2NC(C)=O)NC(=O)CCOCCOCc2ccccc2)OC(COC(C)=O)C(OC(C)=O)C1OC(C)=O]

(1) Under the N₂ atmosphere, NC-4 (2.6 g, 4.7 mmol) is dissolved in DCM (200 mL), the temperature is reduced to 0-5° C. in the ice-water bath, HATU (5.6 g, 14.83 mmol) and DIEA (4.85 g, 37.6 mmol) are added and stirred for 20 minutes.

(2) Then, GN-17 (8.45 g, 15.5 mmol) is added, the temperature is risen to 25° C. and it is stirred and reacted for 4 hours. TLC detection shows that raw materials are basically disappeared.

(3) The saturated ammonium chloride solution (50 mL) is added for quenching, solution is separated, and it is extracted by DCM (100 mL×2).

(4) Organic phases are combined and washed with the saturated salt water (100 mL), and dried with the anhydrous Na₂SO₄.

(5) It is filtered and concentrated to obtain a crude product. After the column chromatography purification (DCM/MeOH=10/1), a white solid TO-23-01 (6.3 g, yield: 63.1%) is obtained.

4. Synthesis of Compound TO-23

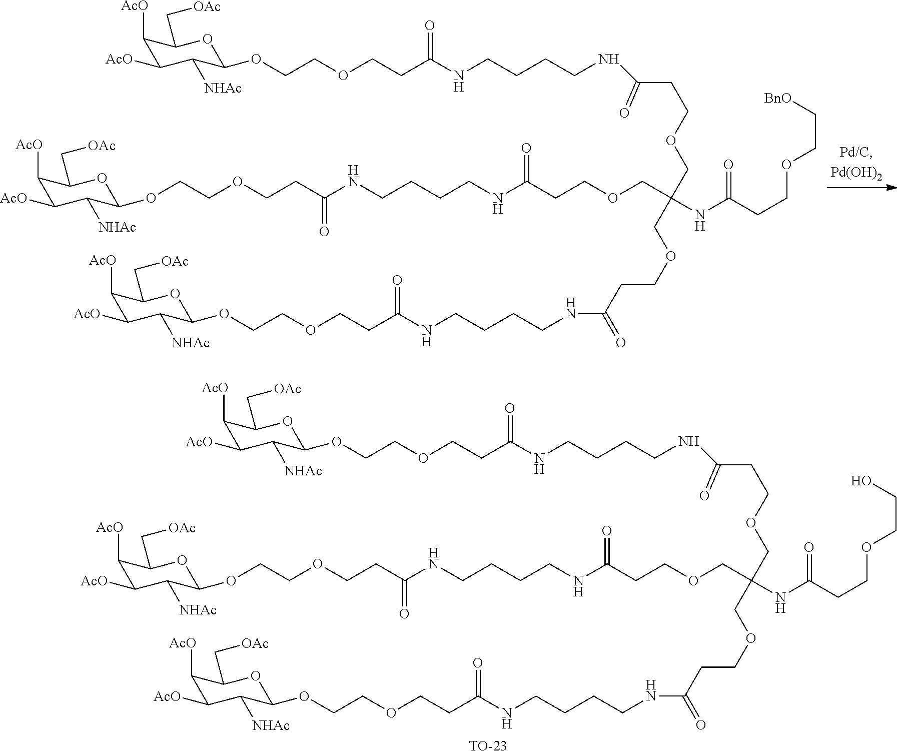

(1) 10% Pd/C (600 mg) and Pd(OH)₂/C (600 mg) are added to MeOH (100 mL) solution of TO-23-01 (6.3 g, 3.0 mmol), it is replaced with H2 for 3 times, and it is stirred and reacted at 25° C. for 3 hours. It is detected by TLC (DCM/MeOH=8/1) that raw materials are basically disappeared.

(2) It is filtered and concentrated to obtain a crude product. After the column chromatography purification (DCM/MeOH/TEA=10/1/0.1), a white solid TO-23 (4.5 g, yield: 75%) is obtained.

¹H NMR (400 MHz, DMSO-d₆) δ 7.88-7.81 (m, 9H), 7.14 (s, 1H), 5.21 (d, J=3.4 Hz, 3H), 4.95 (dd, J=11.2, 3.4 Hz, 3H), 4.53 (d, J=8.5 Hz, 3H), 4.07-3.97 (m, 9H), 3.88 (dt, J=11.0, 9.0 Hz, 3H), 3.77-3.71 (m, 3H), 3.63-3.50 (m, 24H), 3.49-3.41 (m, 8H), 3.38-3.35 (m, 2H), 3.08-2.98 (m, 12H), 2.35-2.25 (m, 14H), 2.10 (s, 9H), 2.00 (s, 9H), 1.89 (s, 9H), 1.78 (s, 9H), 1.40-1.33 (s, 12H).

MS (ESI): m/z [½M+H]+theoretical value 1000.5, measured value 1000.3.

5. Synthesis of Compound TP-23 (Precursor of 1043 Target Linked to siRNA)

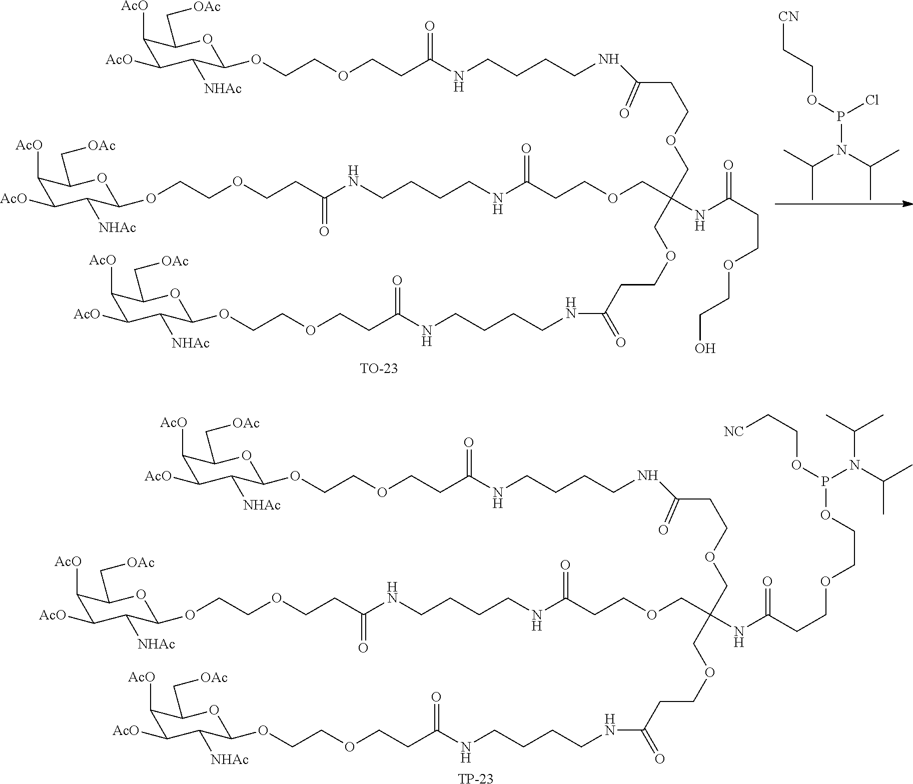

(1) Under the N₂ atmosphere, TO-23 (2.3 g, 1.15 mmol) is dissolved in dry DCM (40 mL), DIEA (0.86 mL, 5.2 mmol) is added, and dry DCM (2 mL) solution of 2-cyanoethyl-N, N-diisopropylchlorophosphoramidite (0.46 mL, 2.1 mmol) is slowly dripped with an injector. It is reacted at 25° C. for 1 hour. It is detected by TLC that raw materials are basically disappeared.

(2) Saturated NaHCO₃ (20 mL) is added for quenching, solution is separated, an organic phase is washed with saturated NaHCO₃ (20 mL) solution and saturated salt water (20 mL), dried with the anhydrous MgSO₄, filtered and concentrated to obtain a crude product. After the column chromatography purification (a silica gel column is alkalized by 1.5% TEA/DCM in advance, DCM/MeOH/TEA=15/1/0.1), a white solid TP-23 (1.8 g, yield: 71.1%) is obtained.

¹H NMR (400 MHz, DMSO-d₆) δ 7.91-7.79 (m, 9H), 7.15 (s, 1H), 5.21 (d, J=3.4 Hz, 3H), 4.95 (dd, J=11.2, 3.4 Hz, 3H), 4.53 (d, J=8.5 Hz, 3H), 4.06-3.97 (m, 9H), 3.88 (dt, J=11.1, 8.9 Hz, 3H), 3.78-3.66 (m, 6H), 3.63-3.41 (m, 36H), 3.07-2.98 (m, 12H), 2.76 (t, J=5.9 Hz, 2H), 2.35-2.24 (m, 14H), 2.10 (s, 9H), 2.00 (s, 9H), 1.89 (s, 9H), 1.78 (s, 9H), 1.40-1.33 (m, 12H), 1.13 (dd, J=6.7, 4.1 Hz, 12H);

³¹P NMR (162 MHz, DMSO-d₆) δ 147.81; and

MS (ESI): m/z[½M+Na]+theoretical value 1122.5, measured value 1122.4.

II. Synthesis of GalNAc Target 1046

According to the following method, a diastereoisomer of TO25 and TP-25 (a precursor of a 1046 target linked to siRNA) is synthesized.

1. Synthesis of Intermediate NC-6-01

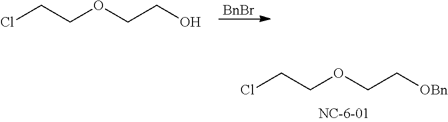 [SMILES: OCCOCCCl ; "BnBr" ; ClCCOCCOCc1ccccc1]

(1) Under the N₂ atmosphere, a dry tetrahydrofuran (THF) (300 mL) is added to a 1000 mL three-necked bottle, the temperature is reduced to 0-5° C. in an ice bath and it is stirred, 60% NaH (14 g, 354.8 mmol) is added in batches, then THF solution (200 mL) of 2-chloroethoxyethanol (40 g, 322.5 mmol) is slowly dripped, the temperature is kept and it is reacted for 30 minutes, then a benzyl bromide (60.3 g, 354.8 mmol) is dropwise added to a reaction bottle, the temperature is risen to 25° C. and it is stirred for 16 hours. It is monitored by TLC that raw materials are basically consumed.

(2) The saturated ammonium chloride solution (150 mL) is slowly dripped for quenching, solution is separated, a aqueous phase is extracted with an ethyl acetate (EtOAc) (100 mL×2), organic phases are combined and washed with the saturated salt water (300 mL), dried with the anhydrous Na₂SO₄, filtered and concentrated to obtain a crude product. The crude product is purified by a silica gel column chromatography (petroleum ether/EtOAc=5/1) to obtain a yellowish oil-like compound NC-6-01 (53 g, yield: 78%).

MS (ESI): m/z [M+H]+theoretical value 215.1, measured value 215.1.

2. Synthesis of Intermediate NC-6-02

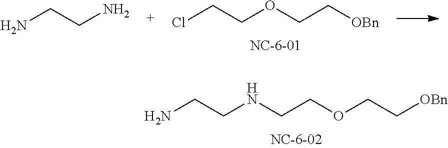

(1) An ethylenediamine (196 g, 3.26 mol) is placed in a 2000 mL three-necked bottle, an acetonitrile (1000 mL), a potassium carbonate (90 g, 0.65 mol) and a sodium iodide (60.6 g, 0.33 mol) are added and stirred. Then, acetonitrile (100 mL) solution of NC-6-01 (70 g, 0.33 mol) is slowly dripped into a reaction bottle, the temperature is risen to 60° C. and it is stirred for 16 hours. It is detected by TLC that raw materials are basically consumed.

(2) A reaction is stopped, it is concentrated, purified water (300 mL) is added, pH is adjusted to 4-5 with a concentrated hydrochloric acid, it is extracted for three times with EtOAc (200 mL×3), a sodium hydroxide solid is added into a aqueous phase so that pH is adjusted to 13-14, it is extracted for three times by DCM (200 mL×3), organic phases are combined and washed with the saturated salt water (300 mL), dried with the anhydrous Na₂SO₄, filtered and concentrated to obtain a yellowish oil-like substance NC-6-02 (69.5 g, 87%).

MS (ESI): m/z [M+H]+theoretical value 239.2, measured value 239.1.

3. Synthesis of Intermediate NC-6-03

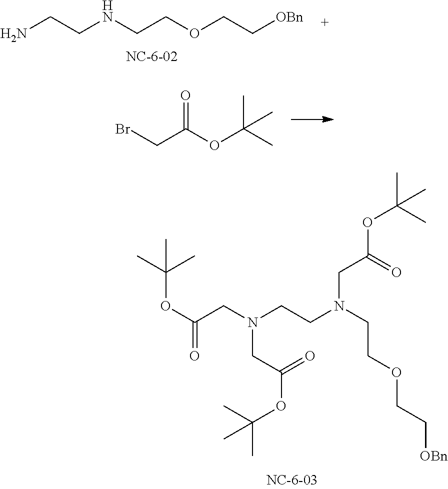

(1) NC-6-02 (69.5 g, 0.29 mol) and tert-butyl bromoacetate (187 g, 0.96 mol) are added to THF (700 mL) and purified water (350 mL), it is stirred, the temperature is reduced below 5° C. in the ice-water bath, and a potassium carbonate (322 g, 2.34 mol) is added. It is stirred and reacted at 25° C. for 14 hours. It is detected by TLC that raw materials are completely converted.

(2) The purified water (300 mL) is added to reaction solution, it is stilly placed and layered, organic phases are separated, a aqueous phase is extracted for two times with EtOAc (200 mL×2), the organic phases are combined, the saturated salt water (500 mL) is added for washing, and it is dried with the anhydrous Na₂SO₄, filtered and concentrated to obtain a yellowish oil-like substance NC-6-03 (201 g).

MS (ESI): m/z [M+H]+theoretical value 581.4, measured value 581.3.

4. Synthesis of Intermediate NC-6

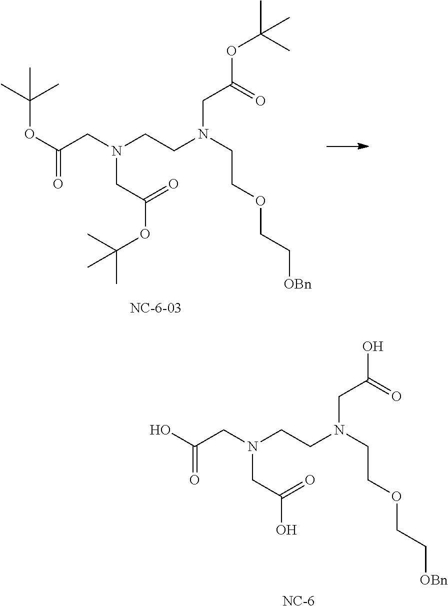 [SMILES: CC(C)(C)OC(=O)CN(CCOCCOCc1ccccc1)CCN(CC(=O)OC(C)(C)C)CC(=O)OC(C)(C)C ; O=C(O)CN(CCOCCOCc1ccccc1)CCN(CC(=O)O)CC(=O)O ; "NC-6"]

(1) NC-6-03 (23 g, 39.6 mmol) is dissolved in 1,4-dioxane (200 mL), a concentrated hydrochloric acid (40 mL) is added, the temperature is risen to 60° C. and it is reacted for 2 hours. It is detected by TLC that raw materials are basically consumed.

(2) It is concentrated, 1,4-dioxane (200 mL) is added again for concentration, to obtain a white solid crude product. The crude product is added to EtOAc (200 mL), it is pulped for 2 hours, suction-filtered to collect a filter cake, and vacuum-dried at 50° C. to obtain a white solid compound NC-6 (22.6 g, 96.9%).

(3) MS (ESI): m/z [M+H]+theoretical value 413.2, measured value 413.1.

5. Synthesis of Intermediate TO-25-01

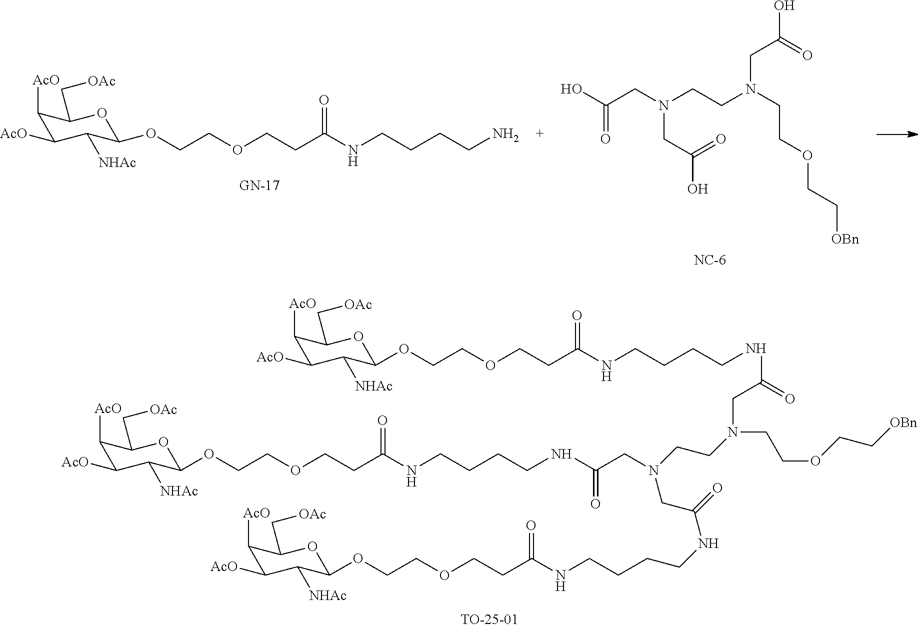 [SMILES: CC(=O)NC1C(OCCOCCC(=O)NCCCCN)OC(COC(C)=O)C(OC(C)=O)C1OC(C)=O ; O=C(O)CN(CCOCCOCc1ccccc1)CCN(CC(=O)O)CC(=O)O ; "NC-6" ; CC(=O)NC1C(OCCOCCC(=O)NCCCCNC(=O)CN(CCOCCOCc2ccccc2)CCN(CC(=O)NCCCCNC(=O)CCOCCOC2OC(COC(C)=O)C(OC(C)=O)C(OC(C)=O)C2NC(C)=O)CC(=O)NCCCCNC(=O)CCOCCOC2OC(COC(C)=O)C(OC(C)=O)C(OC(C)=O)C2NC(C)=O)OC(COC(C)=O)C(OC(C)=O)C1OC(C)=O]

(1) Under the N₂ atmosphere, NC-6 (1.5 g, 3.6 mmol), HBTU (4.5 g, 12.0 mmol) and DIEA (4.75 g, 36 mmol) are added to DCM (50 mL) and stirred for 30 minutes, then DCM (50 mL) solution of GN-17 (6.4 g, 12.0 mmol) and DIEA (4.75 g, 36 mmol) are dropwise added, and stirred at 25° C. for 16 hours. It is detected by a liquid chromatography mass spectrometry (LCMS) that raw materials are basically consumed.

(2) DCM (100 mL) is added for dilution, 1 N of hydrochloric acid solution (80 mL×2) is added to reaction solution for washing, organic phases are combined, and it is washed with the saturated sodium bicarbonate (100 mL), washed with the saturated salt water (100 mL), dried with the anhydrous Na₂SO₄, filtered and concentrated to obtain a crude product. The crude product is purified by the silica gel column chromatography (DCM/MeOH=7/1) to obtain a white solid compound TO-25-01 (4.3 g, yield: 60%).

(3) MS (ESI): m/z [M/2+H]+theoretical value 980.0, measured value 979.9.

6. Synthesis of Intermediate TO-25

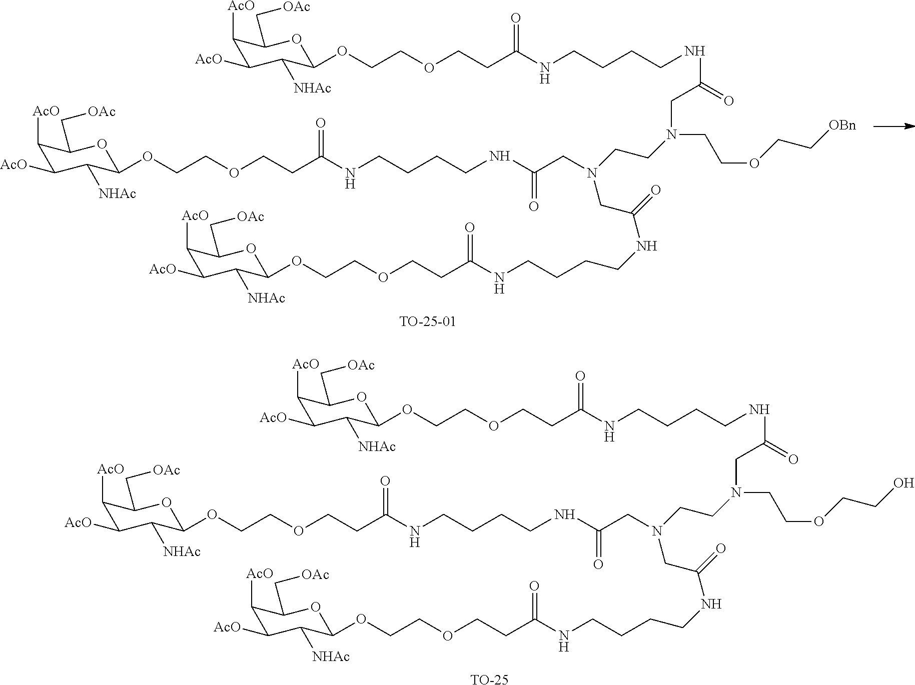

(1) TO-25-01 (4.3 g, 2.2 mmol) is dissolved in methanol (80 mL), 10% palladium carbon (1.0 g) is added, it is replaced with H₂ for three times, and stirred at 25° C. for 2 hours. It is detected by LCMS that raw material are basically disappeared.

(2) It is filtered and concentrated, DCM (20 mL) is added to dissolve, it is slowly dripped into a methyl tert-butyl ether (MTBE) (300 mL), stirred and crystallized for 30 minutes, and suction-filtered, to obtain a white solid compound TO-25 (3.7 g, yield: 90%).

¹H NMR (400 MHz, DMSO-d₆) δ 8.48 (d, J=5.6 Hz, 1H), 8.06 (t, J=5.7 Hz, 2H), 7.85 (dd, J=11.7, 6.8 Hz, 6H), 5.21 (d, J=3.3 Hz, 3H), 4.95 (dd, J=11.2, 3.3 Hz, 3H), 4.53 (d, J=8.5 Hz, 3H), 4.08-3.83 (m, 14H), 3.75 (p, J=4.8 Hz, 5H), 3.68-3.26 (m, 28H), 3.21-2.95 (m, 14H), 2.30 (q, J=7.9, 6.7 Hz, 6H), 1.94-1.78 (m, 36H), 1.41-1.38 (m, 12H); and

MS (ESI): m/z [½M+H]+theoretical value 934.9, measured value 934.8.

7. Synthesis of TP-25 (Precursor of 1046 Target Linked to siRNA)

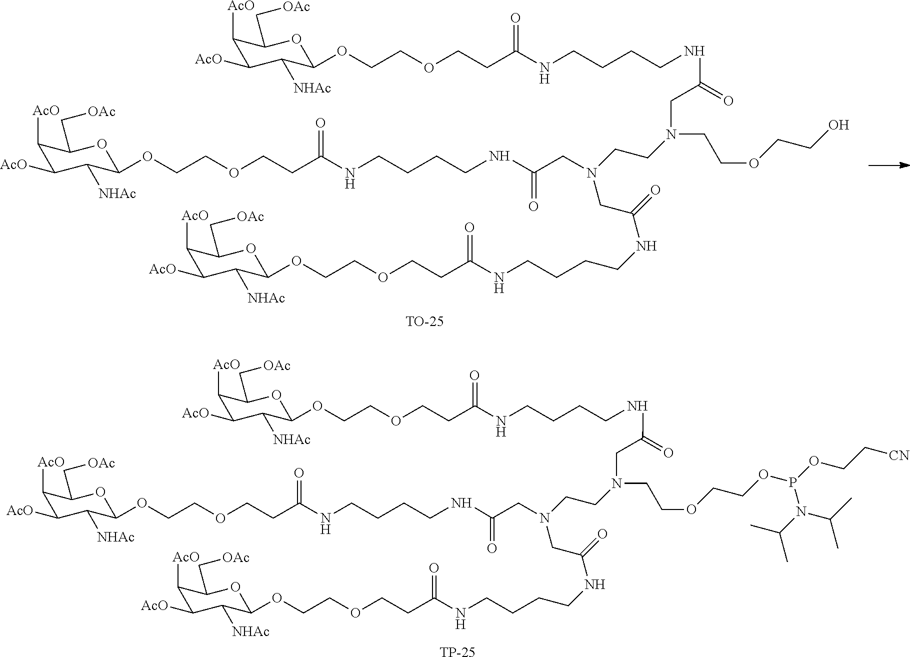 [SMILES: CC(=O)NC1C(OCCOCCC(=O)NCCCCNC(=O)CN(CCOCCO)CCN(CC(=O)NCCCCNC(=O)CCOCCOC2OC(COC(C)=O)C(OC(C)=O)C(OC(C)=O)C2NC(C)=O)CC(=O)NCCCCNC(=O)CCOCCOC2OC(COC(C)=O)C(OC(C)=O)C(OC(C)=O)C2NC(C)=O)OC(COC(C)=O)C(OC(C)=O)C1OC(C)=O ; "TO-25" ; CC(=O)NC1C(OCCOCCC(=O)NCCCCNC(=O)CN(CCOCCOP(OCCC#N)N(C(C)C)C(C)C)CCN(CC(=O)NCCCCNC(=O)CCOCCOC2OC(COC(C)=O)C(OC(C)=O)C(OC(C)=O)C2NC(C)=O)CC(=O)NCCCCNC(=O)CCOCCOC2OC(COC(C)=O)C(OC(C)=O)C(OC(C)=O)C2NC(C)=O)OC(COC(C)=O)C(OC(C)=O)C1OC(C)=O ; "TP-25"]

(1) Under the N₂ atmosphere, TO-25 (700 mg, 0.37 mmol) is dissolved in dry DCM (10 mL), DIEA (0.31 mL, 1.9 mmol) is added, dry DCM (1 mL) solution of 2-cyanoethyl-N,N-diisopropylchlorophosphoramidite (0.19 mL, 0.74 mmol) is slowly dripped with the injector, and it is reacted at 25° C. for 30 minutes. It is detected by TLC that raw materials are basically disappeared.

(2) Saturated NaHCO₃ (10 mL) is added for quenching, it is diluted by DCM (10 mL), solution is separated, an organic phase is washed with saturated NaHCO₃ (10 mL) solution and saturated salt water (10 mL), it is dried with the anhydrous NaSO₄, filtered and concentrated to obtain a crude product. After the column chromatography purification (the silica gel column is alkalized by 1.5% TEA/DCM in advance, DCM/MeOH/TEA=15/1/0.1), a white solid TP-25 (405 mg, yield: 53%) is obtained.

¹H NMR (400 MHz, DMSO-d₆) δ 8.12 (t, J=6.0 Hz, 2H), 7.98-7.75 (m, 7H), 5.21 (d, J=3.4 Hz, 3H), 4.96 (dd, J=11.2, 3.4 Hz, 2H), 4.54 (d, J=8.4 Hz, 2H), 4.02 (q, J=5.3, 4.5 Hz, 9H), 3.95-3.83 (m, 3H), 3.82-3.50 (m, 23H), 3.40-3.26 (m, 4H), 3.12-2.94 (m, 27H), 2.76-2.59 (m, 7H), 2.29 (t, J=6.7 Hz, 5H), 2.11-1.78 (m, 38H), 1.38 (s, 12H), 1.16 (d, J=7.5 Hz, 12H);

³¹P NMR (162 MHz, DMSO-d6) δ 147.97; and

MS (ESI): m/z [½M+Na]+theoretical value 1057.0, measured value 1057.4.

III. Synthesis of GalNAc target 1048

According to the following method, a diastereoisomer of TO26 and TP-26 (a precursor of a 1048 target linked to siRNA) is synthesized.

1. Synthesis of Intermediate GN-18-01

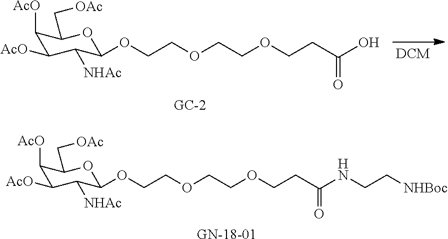 [SMILES: CC(=O)NC1C(OCCOCCOCCC(=O)O)OC(COC(C)=O)C(OC(C)=O)C1OC(C)=O ; "GC-2" ; CC(=O)NC1C(OCCOCCOCCC(=O)NCCNC(=O)OC(C)(C)C)OC(COC(C)=O)C(OC(C)=O)C1OC(C)=O ; "GN-18-01"]

(1) Under the N₂ atmosphere, GC-2 (20.1 g, 39.7 mmol) is dissolved in DCM (200 mL), carbonyldiimidazole (CDI) (7.09 g, 73.7 mmol) is added in batches, it is stirred at 25° C. for 3 hours, then N-Bocethylenediamine (7.0 g, 43.7 mmol) and triethylamine (12.05 g, 119.1 mmol) are added to reaction solution, and it is reacted for 16 hours. LCMS detection shows that raw materials are disappeared.

(2) The saturated sodium bicarbonate solution (200 mL) is added for quenching, solution is separated, a aqueous phase is extracted with DCM (100 mL×3), organic phases are combined, and it is washed with saturated ammonium chloride solution (200 mL) and saturated sodium chloride solution (200 mL), dried with the anhydrous Na₂SO₄, filtered and concentrated to obtain a crude product. The crude product is washed with the methyl tert-butyl ether (100 mL), and an oil-like product is concentrated to obtain a white solid compound GN-18-01 (24.43 g, yield: 95.1%).

MS (ESI): m/z [M+H]+theoretical value 650.3, measured value 650.5.

2. Synthesis of Intermediate GN-18

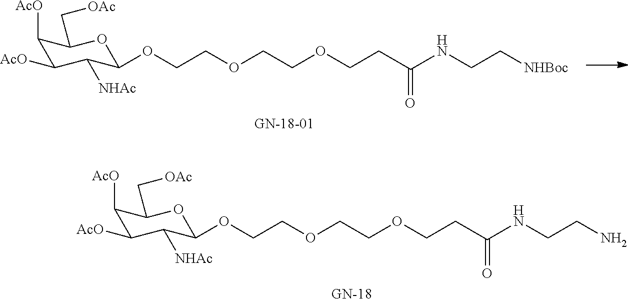 [SMILES: CC(=O)NC1C(OCCOCCOCCC(=O)NCCNC(=O)OC(C)(C)C)OC(COC(C)=O)C(OC(C)=O)C1OC(C)=O ; "GN-18-01" ; CC(=O)NC1C(OCCOCCOCCC(=O)NCCN)OC(COC(C)=O)C(OC(C)=O)C1OC(C)=O ; "GN-18"]

(1) GN-18-01 (45.52 g, 70 mmol) is added to HCl/EtOAc solution (2 N, 500 mL) in batches, and it is stirred at 25° C. for 2 hours. LCMS detection shows that raw materials are disappeared.

(2) A solvent is poured out, and a solid is concentrated to obtain a crude product. The crude product is pulped and purified by the methyl tert-butyl ether (200 mL), it is filtered, and a filter cake is vacuum-dried at 40° C. to obtain a white solid GN-18 (49.6 g).

MS (ESI): m/z [M+H]+theoretical value 550.3, measured value 550.5.

3. Synthesis of Intermediate TO-26-01

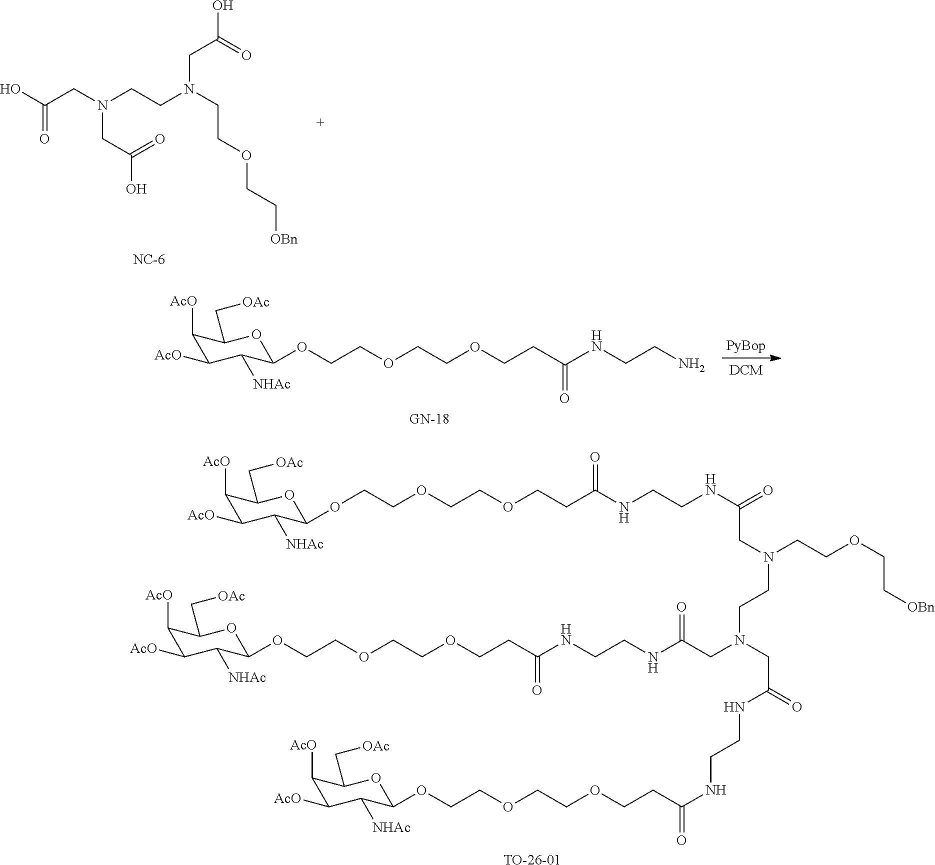 [SMILES: O=C(O)CN(CCOCCOCc1ccccc1)CCN(CC(=O)O)CC(=O)O ; "NC-6" ; CC(=O)NC1C(OCCOCCOCCC(=O)NCCN)OC(COC(C)=O)C(OC(C)=O)C1OC(C)=O ; "GN-18" ; CC(=O)NC1C(OCCOCCOCCC(=O)NCCNC(=O)CN(CCOCCOCc2ccccc2)CCN(CC(=O)NCCNC(=O)CCOCCOCCOC2OC(COC(C)=O)C(OC(C)=O)C(OC(C)=O)C2NC(C)=O)CC(=O)NCCNC(=O)CCOCCOCCOC2OC(COC(C)=O)C(OC(C)=O)C(OC(C)=O)C2NC(C)=O)OC(COC(C)=O)C(OC(C)=O)C1OC(C)=O ; "TO-26-01"]

(1) Under the N₂ atmosphere, NC-6 (1.5 g, 3.6 mmol), hexafluorophosphate (PyBOP) (6.2 g, 12.0 mmol) and DIEA (4.75 g, 36 mmol) are added to DCM (50 mL) and stirred for 30 minutes, then DCM (50 mL) solution of GN-18 (6.6 g, 12.0 mmol) and DIEA (4.75 g, 36 mmol) is dropwise added, and it is stirred at 25° C. for 16 hours. It is detected by LCMS that raw materials are basically consumed.

(2) DCM (100 mL) is added for dilution, 1 N of hydrochloric acid solution (80 mL×2) is added to reaction solution for washing, organic phases are combined, and it is washed with saturated sodium bicarbonate (100 mL) and saturated salt water (100 mL), dried with the anhydrous Na₂SO₄, filtered and concentrated to obtain a crude product. The crude product is purified by the silica gel column chromatography (DCM/MeOH=7/1) to obtain a white solid compound TO-26-01 (4.7 g, yield: 65%).

MS (ESI): m/z [M/2+H]+theoretical value 1004.0, measured value 1004.2.

4. Synthesis of Intermediate TO-26

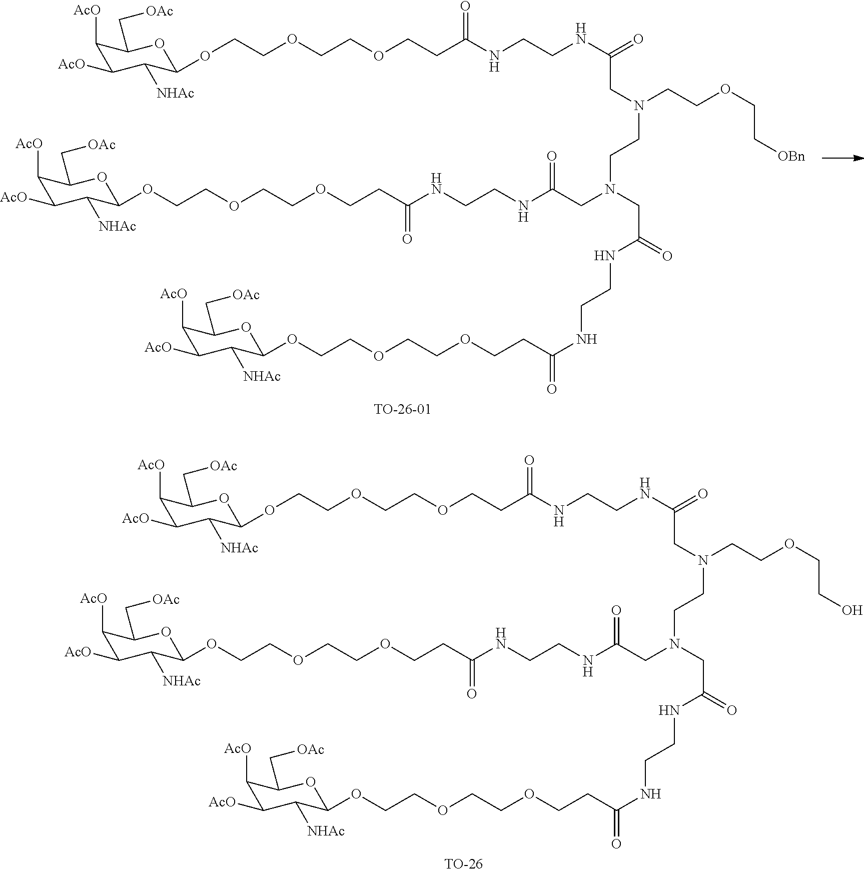 [SMILES: CC(=O)NC1C(OCCOCCOCCC(=O)NCCNC(=O)CN(CCOCCOCc2ccccc2)CCN(CC(=O)NCCNC(=O)CCOCCOCCOC2OC(COC(C)=O)C(OC(C)=O)C(OC(C)=O)C2NC(C)=O)CC(=O)NCCNC(=O)CCOCCOCCOC2OC(COC(C)=O)C(OC(C)=O)C(OC(C)=O)C2NC(C)=O)OC(COC(C)=O)C(OC(C)=O)C1OC(C)=O ; "TO-26-01" ; CC(=O)NC1C(OCCOCCOCCC(=O)NCCNC(=O)CN(CCOCCO)CCN(CC(=O)NCCNC(=O)CCOCCOCCOC2OC(COC(C)=O)C(OC(C)=O)C(OC(C)=O)C2NC(C)=O)CC(=O)NCCNC(=O)CCOCCOCCOC2OC(COC(C)=O)C(OC(C)=O)C(OC(C)=O)C2NC(C)=O)OC(COC(C)=O)C(OC(C)=O)C1OC(C)=O ; "TO-26"]

(1) TO-26-01 (4.0 g, 2.0 mmol) is dissolved in methanol (80 mL), 10% palladium carbon (1.0 g) is added, it is replaced with H₂ for three times, and stirred at 25° C. for 2 hours. It is detected by LCMS that raw materials are basically disappeared.

(2) It is filtered, and concentrated, DCM (20 mL) is added to dissolve, it is slowly dripped into MTBE (200 mL), stirred and crystallized for 30 minutes, and suction-filtered, to obtain a white solid compound TO-26 (3.5 g, yield: 91%).

¹H NMR (400 MHz, DMSO-d₆) δ 8.54 (s, 1H), 8.14 (s, 2H), 7.95-7.92 (m, 3H), 7.84 (d, J=7.8 Hz, 3H), 5.21 (d, J=3.4 Hz, 3H), 4.97 (dd, J=11.2, 3.4 Hz, 3H), 4.54 (d, J=8.5 Hz, 3H), 4.13-3.66 (m, 21H), 3.60-3.44 (m, 37H), 3.14 (d, J=13.8 Hz, 15H), 2.31 (t, J=6.4 Hz, 6H), 2.10 (s, 9H), 2.00 (s, 9H), 1.89 (s, 9H), 1.77 (s, 9H).

MS (ESI): m/z [½M+H]+theoretical value 958.9, measured value 959.1.

5. Synthesis of TP-26 (Precursor of 1048 Target Linked to siRNA)

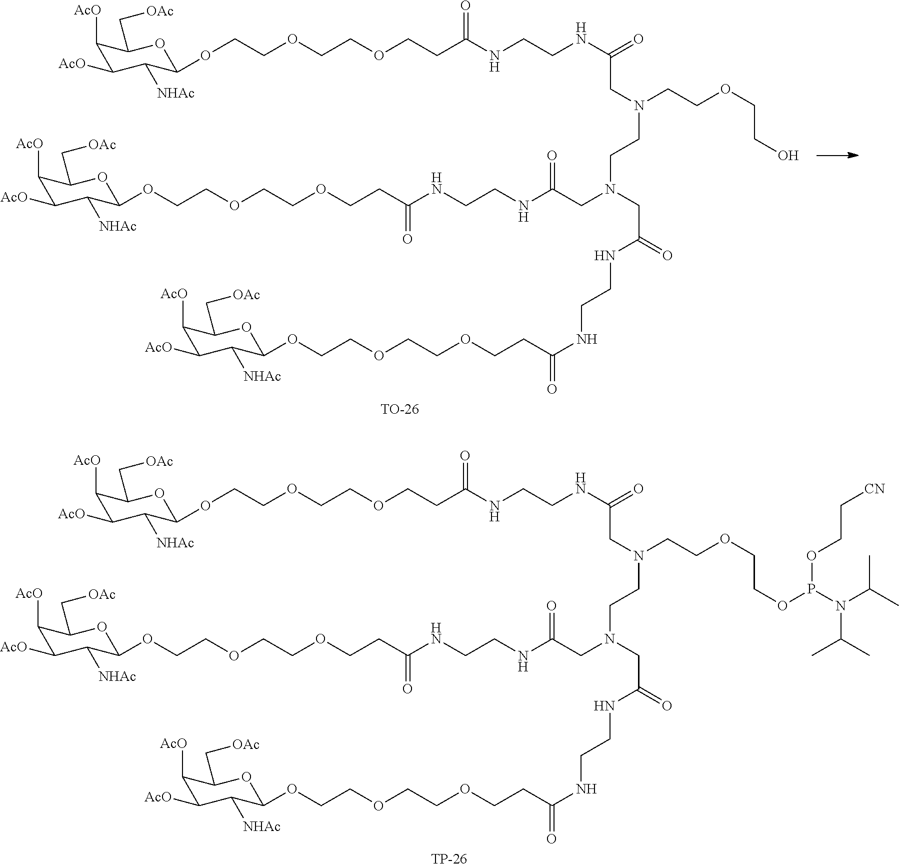 [SMILES: CC(=O)NC1C(OCCOCCOCCC(=O)NCCNC(=O)CN(CCOCCO)CCN(CC(=O)NCCNC(=O)CCOCCOCCOC2OC(COC(C)=O)C(OC(C)=O)C(OC(C)=O)C2NC(C)=O)CC(=O)NCCNC(=O)CCOCCOCCOC2OC(COC(C)=O)C(OC(C)=O)C(OC(C)=O)C2NC(C)=O)OC(COC(C)=O)C(OC(C)=O)C1OC(C)=O ; "TO-26" ; CC(=O)NC1C(OCCOCCOCCC(=O)NCCNC(=O)CN(CCOCCOP(OCCC#N)N(C(C)C)C(C)C)CCN(CC(=O)NCCNC(=O)CCOCCOCCOC2OC(COC(C)=O)C(OC(C)=O)C(OC(C)=O)C2NC(C)=O)CC(=O)NCCNC(=O)CCOCCOCCOC2OC(COC(C)=O)C(OC(C)=O)C(OC(C)=O)C2NC(C)=O)OC(COC(C)=O)C(OC(C)=O)C1OC(C)=O ; "TP-26"]

(1) Under the N₂ atmosphere, TO-26 (900 mg, 0.47 mmol) is dissolved in dry DCM (12 mL), DIEA (0.39 mL, 0.44 mmol) is added, dry DCM (1 mL) solution of 2-cyanoethyl-N, N-diisopropylchlorophosphoramidite (277 mg, 1.17 mmol) is slowly dripped with the injector, and it is reacted at 25° C. for 30 minutes. It is detected by TLC that raw materials are basically disappeared.

(2) Saturated NaHCO₃ (10 mL) is added for quenching, it is diluted by DCM (10 mL), solution is separated, an organic phase is washed with saturated NaHCO₃ (10 mL) solution and saturated salt water (10 mL), dried with the anhydrous NaSO₄, filtered and concentrated to obtain a crude product. After the column chromatography purification (the silica gel column is alkalized by 1.5% TEA/DCM in advance, DCM/MeOH/TEA=15/1/0.1), a white solid TP-26 (600 mg, yield: 60%) is obtained.

¹H NMR (400 MHz, DMSO-d6) ¹H NMR (400 MHz, DMSO-d₆) δ 8.15 (s, 2H), 7.94-7.81 (m, 7H), 5.22 (d, J=3.4 Hz, 3H), 4.97 (dd, J=11.2, 3.4 Hz, 3H), 4.55 (d, J=8.5 Hz, 3H), 4.03 (s, 8H), 3.88 (dt, J=11.2, 8.9 Hz, 3H), 3.81-3.67 (m, 7H), 3.64-3.46 (m, 30H), 3.11 (d, J=13.1 Hz, 19H), 2.76 (t, J=5.9 Hz, 3H), 2.65-2.54 (m, 7H), 2.31 (t, J=6.6 Hz, 7H), 2.11 (s, 9H), 2.00 (s, 9H), 1.89 (s, 9H), 1.77 (s, 9H), 1.13 (d, J=6.8, 12H).

³¹P NMR (162 MHz, DMSO-d6) δ 147.89; and

MS (ESI): m/z ½ [M-i-Pr₂N] theoretical value 1007.9, measured value 1008.2.

Embodiment 3: In Vitro Construction of siRNA Conjugate Coupled by Coupling GalNAc Target

Oligonucleotide sequence portions of an antisense chain and a sense chain of the following RNAi agent double-chain body, as well as linkage between a target ligand and RNA, are all in accordance with phosphite amide coupling technologies reported by J Org. Chem. 2012, 77, 4566-4577; Curr. Protoc. Nucleic Acid Chem., 81, e107, and are synthesized on a solid phase for oligonucleotide synthesis. The target ligands 1046, 1048 and 1043 are all linked to a 5′-end of the siRNA sense chain by a thiophosphate bond.

The form of the target ligands 1046, 1048 and 1043 linked with siRNA is a form of removing a hydroxyl protecting group, and it is specifically as follows:

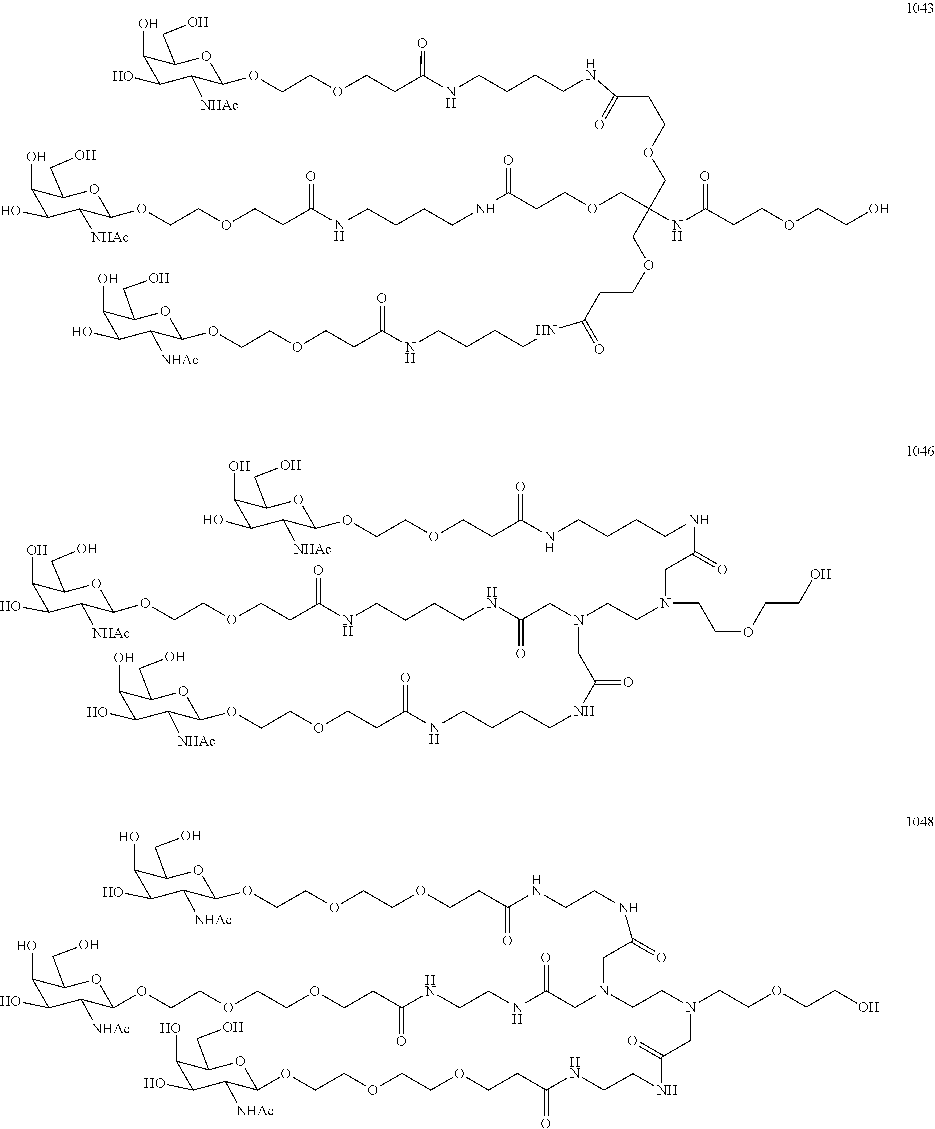 [SMILES: CC(=O)NC1C(OCCOCCC(=O)NCCCCNC(=O)CCOCC(COCCC(=O)NCCCCNC(=O)CCOCCOC2OC(CO)C(O)C(O)C2NC(C)=O)(COCCC(=O)NCCCCNC(=O)CCOCCOC2OC(CO)C(O)C(O)C2NC(C)=O)NC(=O)CCOCCO)OC(CO)C(O)C1O ; CC(=O)NC1C(OCCOCCC(=O)NCCCCNC(=O)CN(CCOCCO)CCN(CC(=O)NCCCCNC(=O)CCOCCOC2OC(CO)C(O)C(O)C2NC(C)=O)CC(=O)NCCCCNC(=O)CCOCCOC2OC(CO)C(O)C(O)C2NC(C)=O)OC(CO)C(O)C1O ; CC(=O)NC1C(OCCOCCOCCC(=O)NCCNC(=O)CN(CCOCCO)CCN(CC(=O)NCCNC(=O)CCOCCOCCOC2OC(CO)C(O)C(O)C2NC(C)=O)CC(=O)NCCNC(=O)CCOCCOCCOC2OC(CO)C(O)C(O)C2NC(C)=O)OC(CO)C(O)C1O]

The structures of the target ligands after being linked with siRNA are as follows:

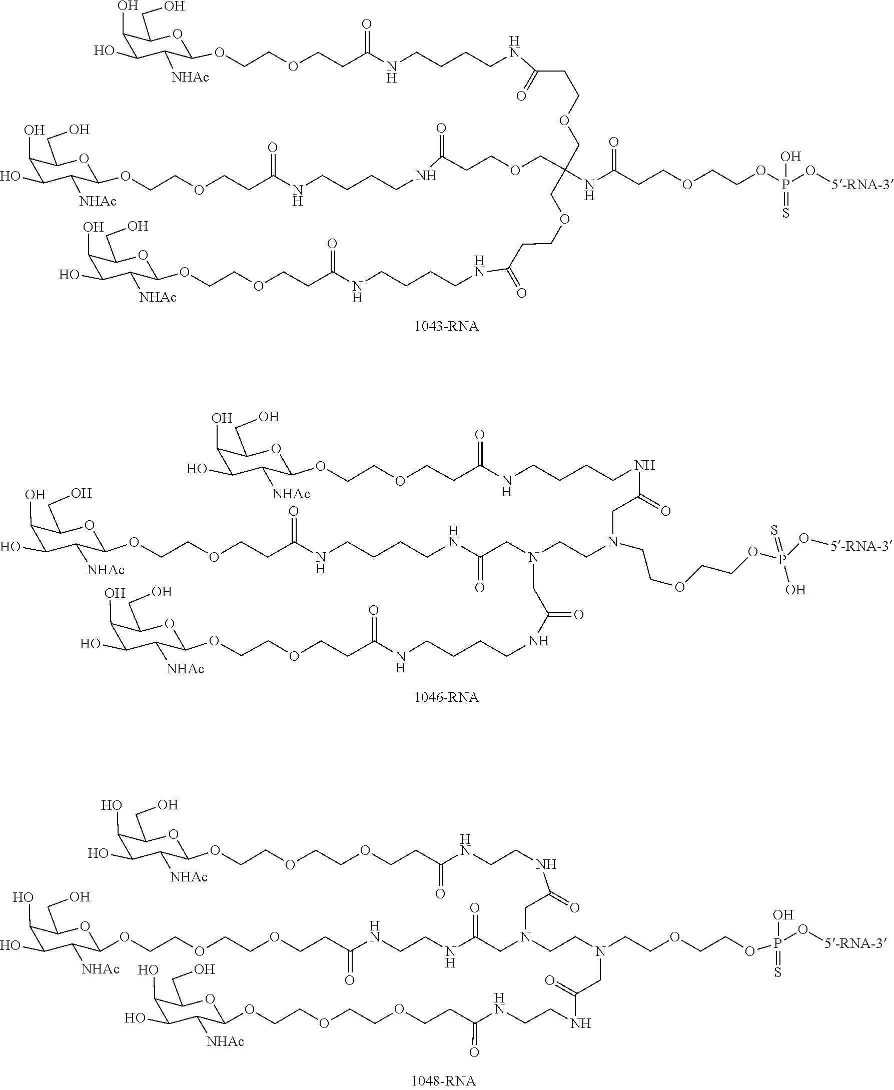

The synthesized GalNAc-siRNA conjugate is described in FIG. 3, herein the structure of the conjugate in the second column includes three portions. For example, the structure of G1043-S2A2-A265 is: the 1043 target is linked with the 5′-end of the siRNA sense chain numbered as A265 by the thiophosphate bond, and S2A2 is the type of modification to siRNA of A265. The specific modification groups and modification modes are as follows.

In the nucleic acid sequence, Ao represents an adenosine, Uo represents a uridine, Go represents a guanosine, and Co represents a cytosine, and there is no symbol between directly adjacent nucleotides, it is indicated that it is linked by a normal phosphate bond.

DNA: A,G,C,T (A represents 2′-deoxyadenosine, T represents 2′-deoxythymidine, G represents 2′-deoxyguanosine, and C represents 2′-deoxycytidine).

2′-F: af,gf,cF,uF (aF represents 2′-fluoroadenine nucleoside, uF represents 2′-fluorouracil nucleoside, gF represents 2′-fluoroguanine nucleoside, and cF represents 2′-fluorocytosine nucleoside).

2′-OMe: aM, gM, cM, uM (aM represents 2′-O-methyladenine nucleoside, uM represents 2′-O-methyluracil nucleoside, gM represents 2′-O-methylguanine nucleoside, and cM represents 2′-O-methylcytosine nucleoside).

*: represents that it is linked by a thiophosphate bond.

y and z in the sequence represent the position of the target.

Embodiment 4: Activity Test of Conjugate by In Vitro Cell Model (Hep 3B Cell)

A human hepatoma Hep3B cell (Shanghai Cell Bank, Chinese Academy of Sciences) is cultured in DMEM (Gibco, US) supplemented with 10% FBS (Gibco, US) under conditions of 37° C. and 5% CO₂ (il60, Thermo Fisher). On the day of a transfection experiment, the cells are digested with 0.25% Trysin (Gibco, US), counted and inoculated on a 24-well plate in the density of 450 μL/well and 50000 cells/well. Subsequently, a test sample is added in a lipofectmine2000 (Thermo Fisher) transfection mode. It is transfected according to a standard flow of RNAiMAX reagent instructions, and the final siRNA concentration is 10 nM/1 nM/0.5 nM/0.25 nM/0.1 nM/0.05 nM/0.01 nM. In a transfection group, siNC is taken as a negative control, and its sequence is as follows.

Sense chain (sense):
(SEQ ID NO. 309)
5′-UUCCGAACGUGUCACGUTT-3′
Antisense chain (antisense):
(SEQ ID NO. 310)
5′-ACGUGACACGUUCGGAGAATT-3′.

After 24 h, the total RNA of the cells is extracted, and the expression conditions of the ANGPTL3 mRNA sequence in the cells are detected by the quantitative real-time PCR, herein PCR primers used to amplify internal reference genes PPIB and ANGPTL3 are shown in Table 1.

The activity test results (EC₅₀ value) of each conjugate are shown in FIG. 4.

The EC₅₀ value is calculated by using non-linear regression of graphpad prism, to express the amount of the conjugate used to inhibit a half of the expression quantity of the target ANGPTL3mRNA.

It may be seen from the results that the selected conjugates show good results in reducing the relative expression level of ANGPTL3 in an experiment of the in vitro activity test.

Embodiment 5: Construction of AAV-hANGPTL3 Mouse Model and Drug Administration Test

Basic information of experimental animals:

The experimental animals are purchased from Jinan Pengyue Experimental Animal Breeding Co., Ltd., which are specific pathogen free (SPF) animals. Before drug administration, the above mice are weighed and statuses are observed, and the animals with uniform weight and no abnormal status are selected for subsequent experiments.

| Species | Gender | Age | Weight | Source |
|---|---|---|---|---|
| C57 mouse | Male | 4 weeks | 20 ± 2 g | Jinan Pengyue |

Feeding conditions: non-SPF feeding conditions. Under normal feeding conditions, the animals may eat and drink freely. After the animals are purchased, the experiment is started after 3-7 days of adaptive culture.

Modeling and administration: each mouse is injected with 2.5*10\hat{ }11 titers of virus solution (100 μL) by a tail vein. After 7 days, the experimental animals are randomly grouped, and each test substance is administered subcutaneously at a dose of 5 mg/kg. In 72 hours after the drug administration, the animals are sacrificed by cervical dislocation, and liver tissues are taken for RNA extraction and quantification.

Results of each conjugate are shown in Table 3.

TABLE 3
Drug administration test results
of mouse model for each conjugate

| | hANGPTL3relative expression level | | |
|---|---|---|---|
| Test | Average | Standard | N (number |
| substance | value | deviation | of animals) |
| PBS control | 1 | 0.24 | 6 |
| NPD006s-129 | 0.47 | 0.12 | 6 |
| NPD006s-130 | 0.44 | 0.22 | 5 |
| NPD006s-131 | 0.34 | 0.07 | 4 |
| NPD006s-132 | 0.41 | 0.16 | 5 |
| NPD006s-133 | 0.45 | 0.15 | 6 |
| NPD006s-134 | 0.55 | 0.12 | 5 |
| NPD006s-135 | 0.79 | 0.2 | 5 |
| NPD006s-136 | 0.55 | 0.14 | 6 |
| NPD006s-137 | 0.47 | 0.17 | 4 |
| NPD006s-138 | 0.61 | 0.43 | 5 |
| NPD006s-139 | 0.74 | 0.09 | 5 |
| NPD006s-140 | 0.35 | 0.11 | 5 |
| NPD006s-141 | 0.52 | 0.28 | 5 |
| NPD006s-143 | 0.6 | 0.09 | 5 |
| NPD006s-144 | 0.52 | 0.07 | 5 |
| NPD006s-145 | 0.46 | 0.07 | 4 |
| NPD006s-146 | 0.62 | 0.25 | 5 |
| NPD006s-147 | 0.39 | 0.12 | 5 |
| NPD006s-148 | 0.51 | 0.14 | 5 |
| NPD006s-151 | 0.40 | 0.31 | 5 |
| NPD006s-167 | 0.47 | 0.14 | 5 |
| NPD006s-168 | 0.47 | 0.26 | 5 |
| NPD006s-169 | 0.58 | 0.29 | 5 |

It may be seen from the results that the selected conjugates also show the good results in reducing the relative expression level of ANGPTL3 in the experiment of the in vivo activity test.

In descriptions of this description, the descriptions of reference terms such as “one embodiment”, “some embodiments”, “examples”, “specific examples”, or “some examples” means that specific features, structures, materials, or characteristics described in combination with this embodiment or example are included in at least one embodiment or example of the present disclosure. In this description, the schematic expressions of the above terms need not refer to the same embodiments or examples. Furthermore, the specific features, structures, materials, or characteristics described may be combined in an appropriate manner in any one or more embodiments or examples. In addition, those skilled in the art may incorporate and combine different embodiments or examples described in this description and the characteristics of the different embodiments or examples in the case without conflicting.

Although the embodiments of the present disclosure are already shown and described above, it may be understood that the above embodiments are exemplary, and may not be understood as limitation to the present disclosure. Those of ordinary skill in the art may change, modify, replace and transform the above embodiments within the scope of the present disclosure.

What is claimed is:

1. A compound, wherein the compound has any one of the following structures:

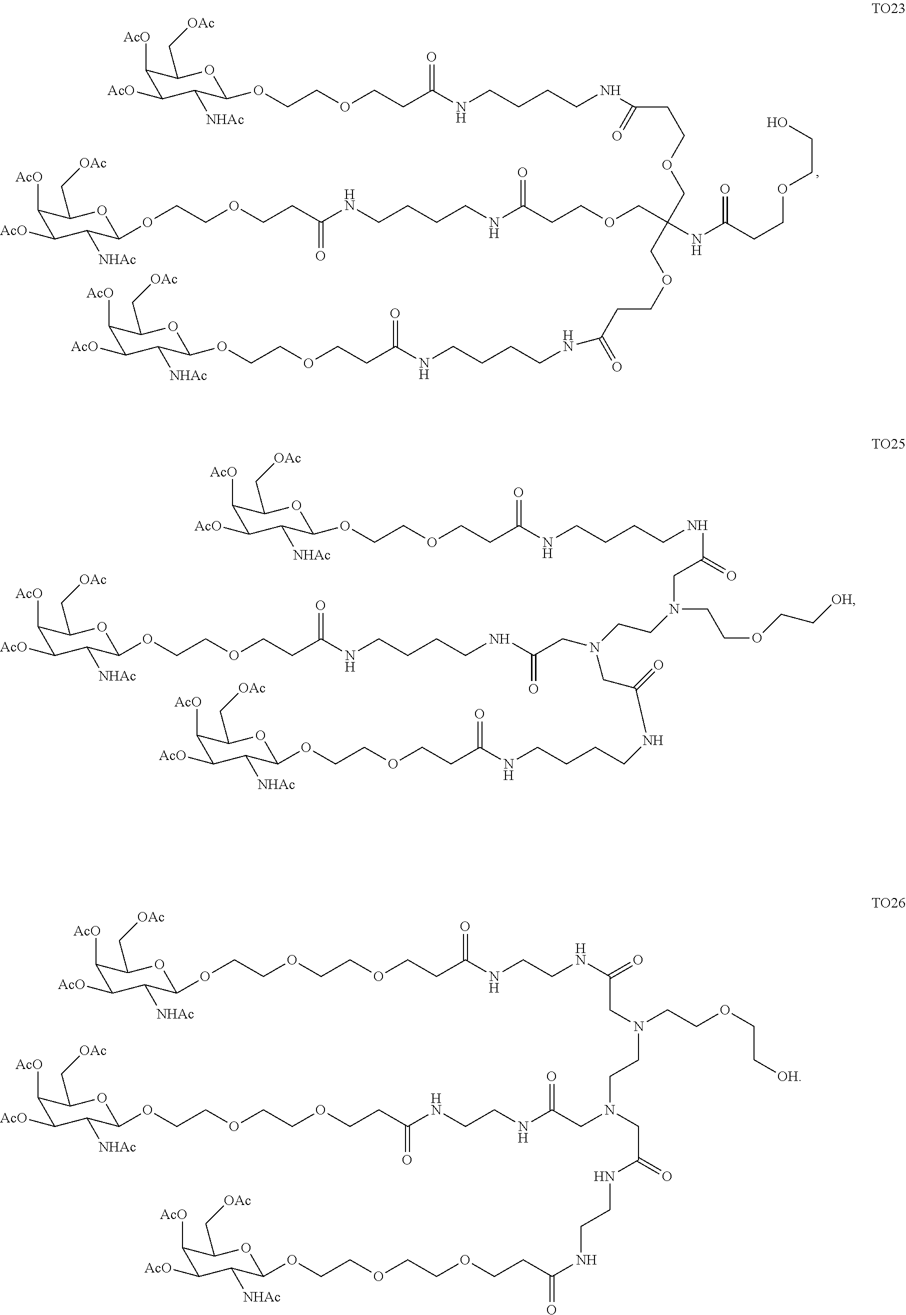

2. A compound, wherein the compound is obtained from the compound according to claim 1 by removing a hydroxyl protecting group, and has one of the following structures:

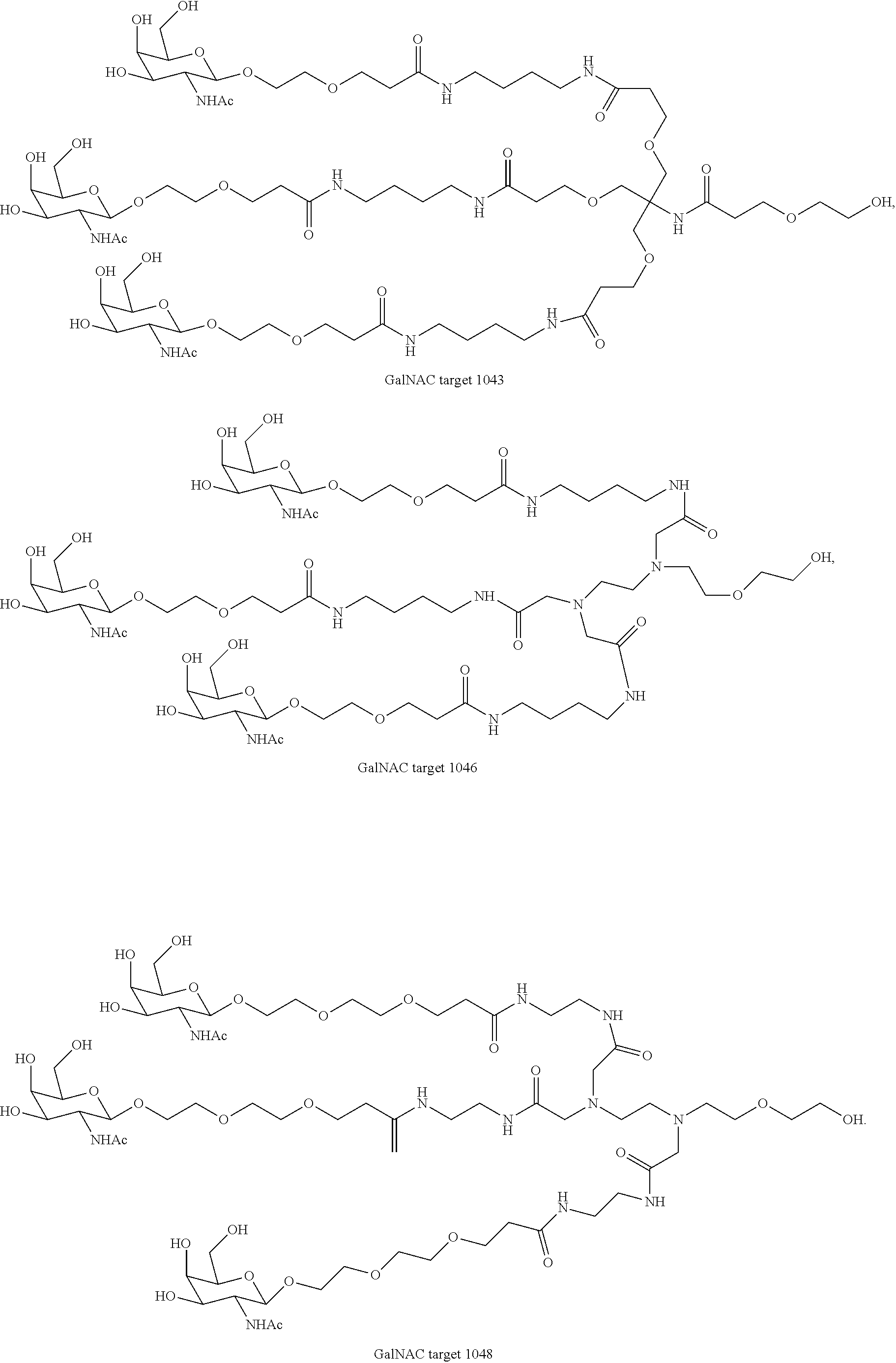 [SMILES: CC(=O)NC1C(OCCOCCC(=O)NCCCCNC(=O)CCOCC(COCCC(=O)NCCCCNC(=O)CCOCCOC2OC(CO)C(O)C(O)C2NC(C)=O)(COCCC(=O)NCCCCNC(=O)CCOCCOC2OC(CO)C(O)C(O)C2NC(C)=O)NC(=O)CCOCCO)OC(CO)C(O)C1O ; CC(=O)NC1C(OCCOCCC(=O)NCCCCNC(=O)CN(CCOCCO)CCN(CC(=O)NCCCCNC(=O)CCOCCOC2OC(CO)C(O)C(O)C2NC(C)=O)CC(=O)NCCCCNC(=O)CCOCCOC2OC(CO)C(O)C(O)C2NC(C)=O)OC(CO)C(O)C1O ; C=C(CCOCCOCCOC1OC(CO)C(O)C(O)C1NC(C)=O)NCCNC(=O)CN(CCN(CCOCCO)CC(=O)NCCNC(=O)CCOCCOCCOC1OC(CO)C(O)C(O)C1NC(C)=O)CC(=O)NCCNC(=O)CCOCCOCCOC1OC(CO)C(O)C(O)C1NC(C)=O]

3. An application of the compound according to claim 1 in preparation of a siRNA conjugate.

4. The application according to claim 3, wherein the compound is linked with siRNA as a target ligand.

5. The application according to claim 4, wherein siRNA in the siRNA conjugate comprises a sense chain and an antisense chain, and the antisense chain comprises a complementary region complementary-paired to the sense chain, wherein the sense chain is selected from a nucleotide sequence that is not more than 5 nucleotides different from a nucleotide sequence of each chain in SEQ ID NO: 1-SEQ ID NO: 154, and the antisense chain is selected from a nucleotide sequence that is not more than 5 nucleotides different from a nucleotide sequence of each chain in SEQ ID NO: 155-SEQ ID NO: 308.

6. The application according to claim 5, wherein the siRNA comprises at least one modified nucleotide;
the modified nucleotide is selected from at least one of the following:
a 5′-thiophosphate based nucleotide, a 5-methylcytosine nucleotide, a 2′-O-methyl modified nucleotide, a 2′-O-2-methoxyethyl modified nucleotide, a 2′-fluoro modified nucleotide, a 3′-nitrogen substituted modified nucleotide, a 2′-deoxy-2′-fluoro modified nucleotide, a 2′-deoxy modified nucleotide, a locked nucleotide, a de-base nucleotide, a 2′-amino modified nucleotide, a morpholinonucleotide, a polypeptide nucleotide, an amino phosphate, and a nucleotide comprising a non-natural base.

7. The application according to claim 6, wherein the siRNA is covalently linked with the target ligand; the target ligand is linked to the sense chain in the siRNA; and the target ligand is linked with a 5′-end of the sense chain in the siRNAby a thiophosphate bond.

8. An application of the compound according to claim 2 in preparation of a siRNA conjugate.

9. The application according to claim 8, wherein the compound is linked with siRNA as a target ligand.

10. The application according to claim 9, wherein siRNA in the siRNA conjugate comprises a sense chain and an antisense chain, and the antisense chain comprises a complementary region complementary-paired to the sense chain, wherein the sense chain is selected from a nucleotide sequence that is not more than 5 nucleotides different from a nucleotide sequence of each chain in SEQ ID NO: 1-SEQ ID NO: 154, and the antisense chain is selected from a nucleotide sequence that is not more than 5 nucleotides different from a nucleotide sequence of each chain in SEQ ID NO: 155-SEQ ID NO: 308.

11. The application according to claim 10, wherein the siRNA comprises at least one modified nucleotide;
the modified nucleotide is selected from at least one of the following:
a 5′-thiophosphate based nucleotide, a 5-methylcytosine nucleotide, a 2′-O-methyl modified nucleotide, a 2′-O-2-methoxyethyl modified nucleotide, a 2′-fluoro modified nucleotide, a 3′-nitrogen substituted modified nucleotide, a 2′-deoxy-2′-fluoro modified nucleotide, a 2′-deoxy modified nucleotide, a locked nucleotide, a de-base nucleotide, a 2′-amino modified nucleotide, a morpholinonucleotide, a polypeptide nucleotide, an amino phosphate, and a nucleotide comprising a non-natural base.

12. The application according to claim 11, wherein the siRNA is covalently linked with the target ligand; the target ligand is linked to the sense chain in the siRNA; and the target ligand is linked with a 5′-end of the sense chain in the siRNA by a thiophosphate bond.

13. A preparation method for asiRNA conjugate, comprising using the compound according to claim 1 as a target ligand to covalently link with siRNA.

14. The method according to claim 13, wherein the compound firstly forms an intermediate and then covalently links with siRNA, and the intermediate is selected from one of the following structures:

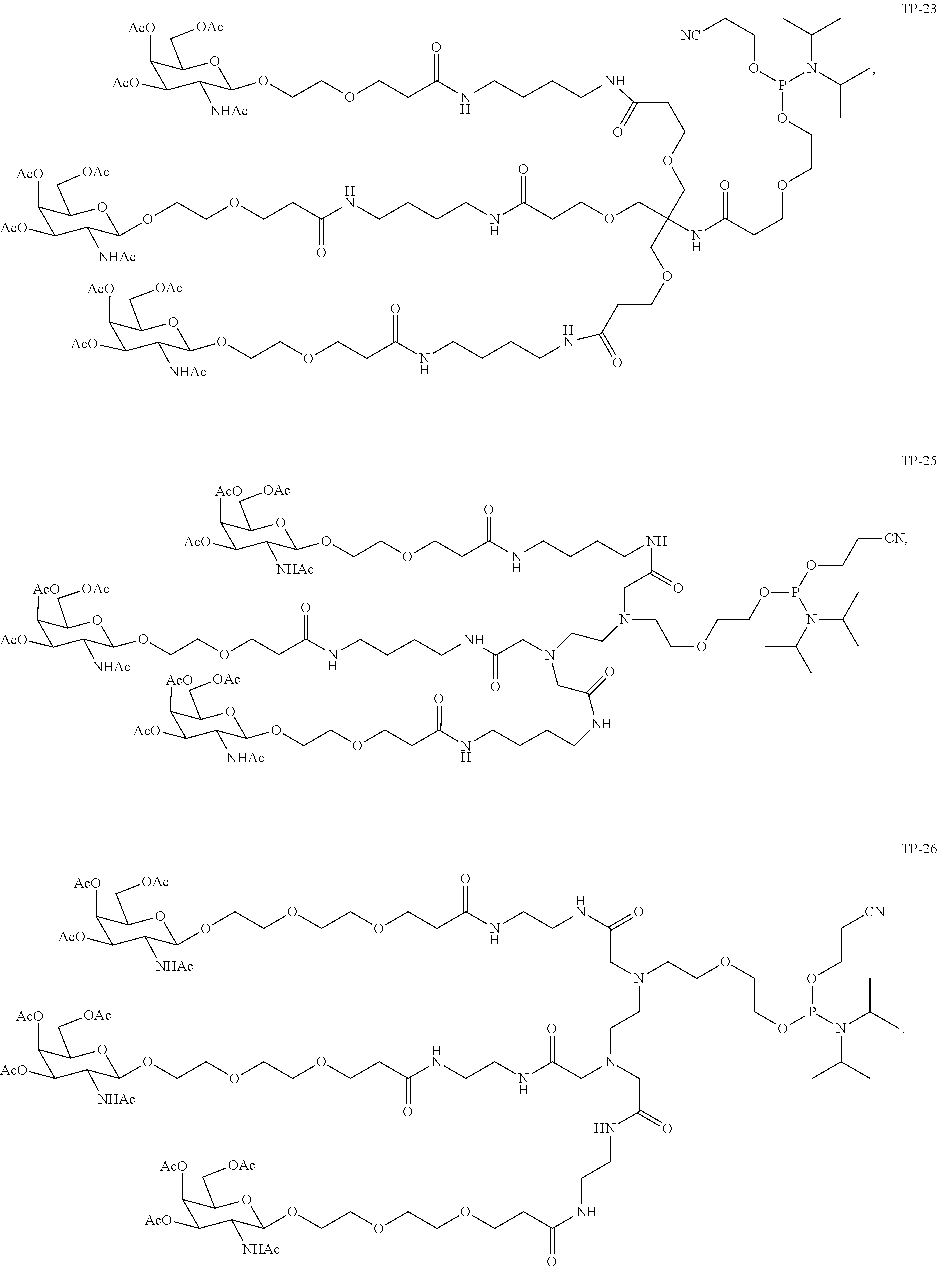

15. A preparation method for asiRNA conjugate, comprising using the compound according to claim 2 as a target ligand to covalently link with siRNA.

16. The method according to claim 15, wherein the compound firstly forms an intermediate and then covalently links with siRNA, and the intermediate is selected from one of the following structures:

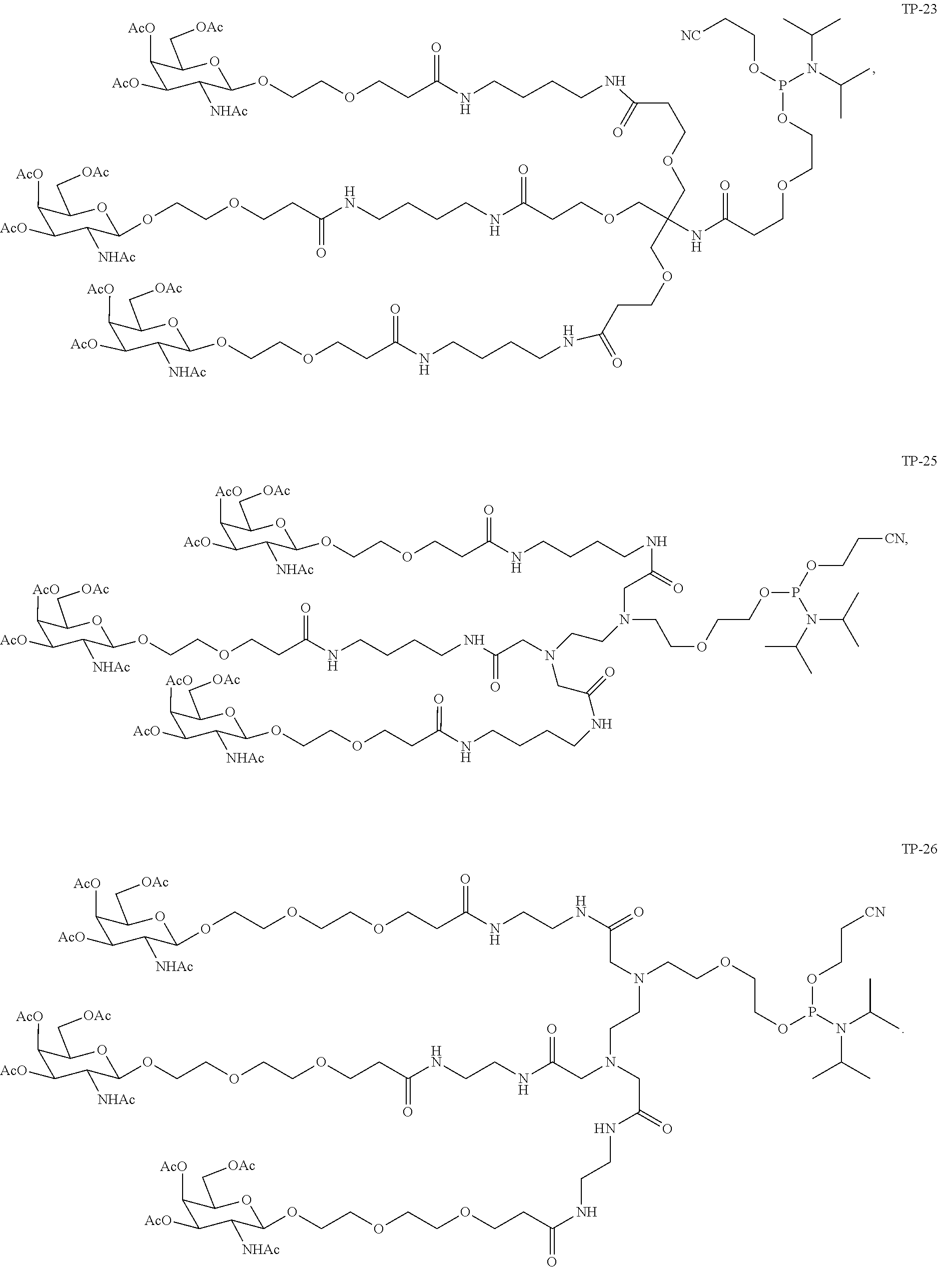

17. An intermediate for preparing a siRNA conjugate, wherein it is selected from one of the following structures:

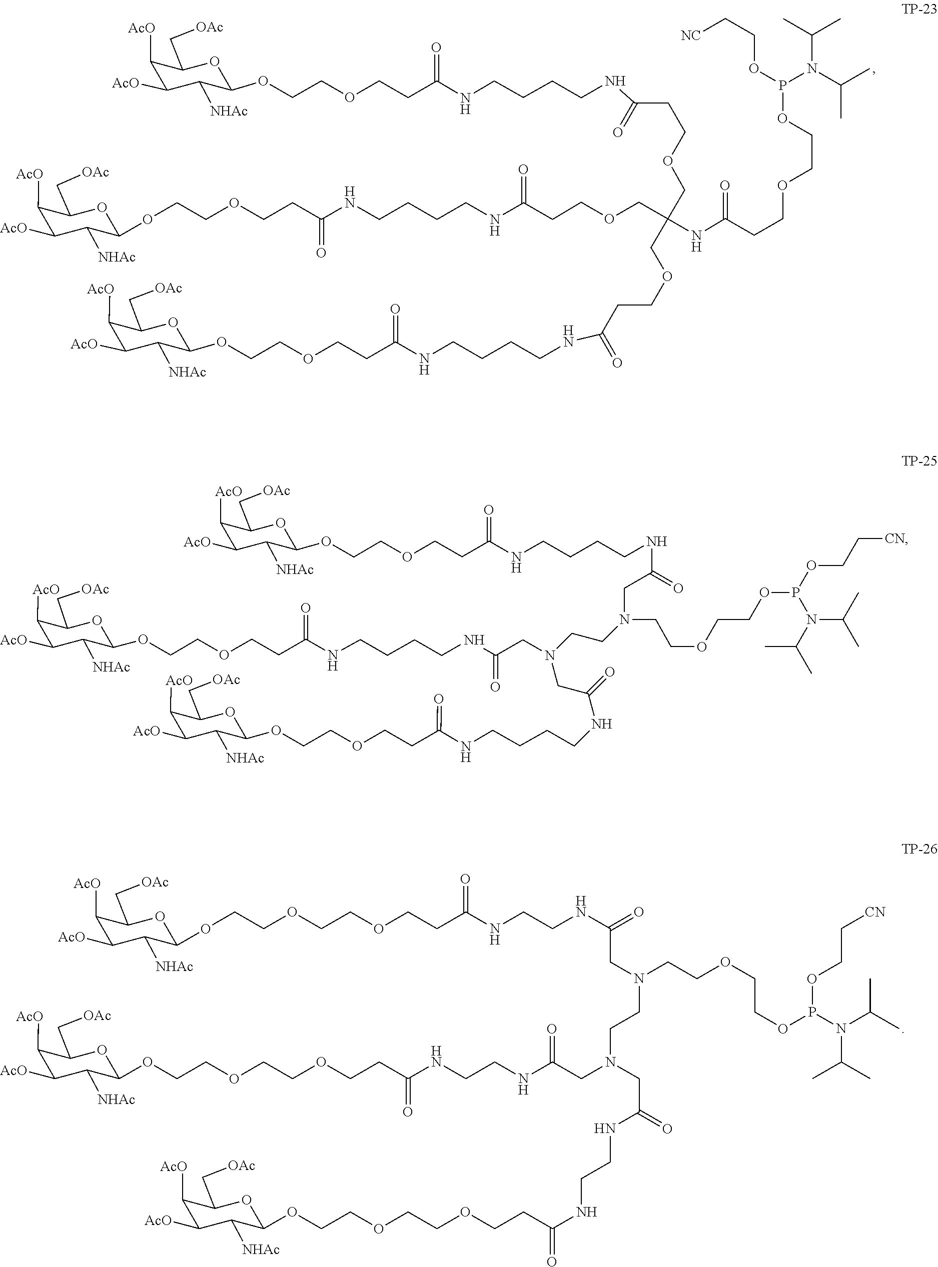

18. An application of the compound according to claim 1 in preparation of a drug or kit, wherein the drug or kit is used to inhibit the expression of the ANGPTL3 gene.

19. The application according to claim 18, wherein the drug or kit is used to prevent and/or treat a dyslipidemia disease.

20. The application according to claim 19, wherein the dyslipidemia disease includes hyperlipidemia and hypertriglyceridemia.


SEQUENCE LISTING (310 sequences)

SEQ ID NO: 1 - RNA, 21 nt, synthetic construct
  source 1..21: mol_type = other RNA
cagaattgatcaagacaattc

SEQ ID NO: 2 - RNA, 21 nt, synthetic construct
  source 1..21: mol_type = other RNA
cagaagtaacttcacttaaaa

SEQ ID NO: 3 - RNA, 21 nt, synthetic construct
  source 1..21: mol_type = other RNA
cacttaaaacttttgtagaaa

SEQ ID NO: 4 - RNA, 21 nt, synthetic construct
  source 1..21: mol_type = other RNA
cttaaaacttttgtagaaaaa

SEQ ID NO: 5 - RNA, 21 nt, synthetic construct
  source 1..21: mol_type = other RNA
gaactactccctttcttcagt

SEQ ID NO: 6 - RNA, 21 nt, synthetic construct
  source 1..21: mol_type = other RNA
catgtctactgtgatgttata

SEQ ID NO: 7 - RNA, 21 nt, synthetic construct
  source 1..21: mol_type = other RNA
gcaatctaattatgttttacg

SEQ ID NO: 8 - RNA, 21 nt, synthetic construct
  source 1..21: mol_type = other RNA
ctaattatgttttacgaattg

SEQ ID NO: 9 - RNA, 21 nt, synthetic construct
  source 1..21: mol_type = other RNA
ccaactatacgctacatctag

SEQ ID NO: 10 - RNA, 21 nt, synthetic construct
  source 1..21: mol_type = other RNA
aagctccttctttttattgtt

SEQ ID NO: 11 - RNA, 21 nt, synthetic construct
  source 1..21: mol_type = other RNA
ttcctctagttatttcctcca

SEQ ID NO: 12 - RNA, 21 nt, synthetic construct
  source 1..21: mol_type = other RNA
ctctagttatttcctccagaa

SEQ ID NO: 13 - RNA, 21 nt, synthetic construct
  source 1..21: mol_type = other RNA
ttcctccagaattgatcaaga

SEQ ID NO: 14 - RNA, 21 nt, synthetic construct
  source 1..21: mol_type = other RNA
ctccagaattgatcaagacaa

SEQ ID NO: 15 - RNA, 21 nt, synthetic construct
  source 1..21: mol_type = other RNA
tccagaattgatcaagacaat

SEQ ID NO: 16 - RNA, 21 nt, synthetic construct
  source 1..21: mol_type = other RNA
ttgatcaagacaattcatcat

SEQ ID NO: 17 - RNA, 21 nt, synthetic construct
  source 1..21: mol_type = other RNA
aagacaattcatcatttgatt

SEQ ID NO: 18 - RNA, 21 nt, synthetic construct
  source 1..21: mol_type = other RNA
gacaattcatcatttgattct

SEQ ID NO: 19 - RNA, 21 nt, synthetic construct
  source 1..21: mol_type = other RNA
ttagacgatgtaaaaatttta

SEQ ID NO: 20 - RNA, 21 nt, synthetic construct
  source 1..21: mol_type = other RNA
tccataagacgaagggccaaa

SEQ ID NO: 21 - RNA, 21 nt, synthetic construct
  source 1..21: mol_type = other RNA
gacgaagggccaaattaatga

SEQ ID NO: 22 - RNA, 21 nt, synthetic construct
  source 1..21: mol_type = other RNA
atgacatatttcaaaaactca

SEQ ID NO: 23 - RNA, 21 nt, synthetic construct
  source 1..21: mol_type = other RNA
tgacatatttcaaaaactcaa

SEQ ID NO: 24 - RNA, 21 nt, synthetic construct
  source 1..21: mol_type = other RNA
gtggcatgatgagtgtggaga

SEQ ID NO: 25 - RNA, 21 nt, synthetic construct
  source 1..21: mol_type = other RNA
atcagtctttttatgatctat

SEQ ID NO: 26 - RNA, 21 nt, synthetic construct
  source 1..21: mol_type = other RNA
cgctgcaaaccagtgaaatca

SEQ ID NO: 27 - RNA, 21 nt, synthetic construct
  source 1..21: mol_type = other RNA
ctgcaaaccagtgaaatcaaa

SEQ ID NO: 28 - RNA, 21 nt, synthetic construct
  source 1..21: mol_type = other RNA
aaccagtgaaatcaaagaaga

SEQ ID NO: 29 - RNA, 21 nt, synthetic construct
  source 1..21: mol_type = other RNA
accagtgaaatcaaagaagaa

SEQ ID NO: 30 - RNA, 21 nt, synthetic construct
  source 1..21: mol_type = other RNA
cagtgaaatcaaagaagaaga

SEQ ID NO: 31 - RNA, 21 nt, synthetic construct
  source 1..21: mol_type = other RNA
tgaaatcaaagaagaagaaaa

SEQ ID NO: 32 - RNA, 21 nt, synthetic construct
  source 1..21: mol_type = other RNA
atcaaagaagaagaaaaggaa

SEQ ID NO: 33 - RNA, 21 nt, synthetic construct
  source 1..21: mol_type = other RNA
aagaagaaaaggaactgagaa

SEQ ID NO: 34 - RNA, 21 nt, synthetic construct
  source 1..21: mol_type = other RNA
aagaaaaggaactgagaagaa

SEQ ID NO: 35 - RNA, 21 nt, synthetic construct
  source 1..21: mol_type = other RNA
aaggaactgagaagaactaca

SEQ ID NO: 36 - RNA, 21 nt, synthetic construct
  source 1..21: mol_type = other RNA
aggaactgagaagaactacat

SEQ ID NO: 37 - RNA, 21 nt, synthetic construct
  source 1..21: mol_type = other RNA
aactgagaagaactacatata

SEQ ID NO: 38 - RNA, 21 nt, synthetic construct
  source 1..21: mol_type = other RNA
actgagaagaactacatataa

SEQ ID NO: 39 - RNA, 21 nt, synthetic construct
  source 1..21: mol_type = other RNA
caagtcaaaaatgaagaggta

SEQ ID NO: 40 - RNA, 21 nt, synthetic construct
  source 1..21: mol_type = other RNA
atgaagaggtaaagaatatgt

SEQ ID NO: 41 - RNA, 21 nt, synthetic construct
  source 1..21: mol_type = other RNA
atgtcacttgaactcaactca

SEQ ID NO: 42 - RNA, 21 nt, synthetic construct
  source 1..21: mol_type = other RNA
ctcctagaagaaaaaattcta

SEQ ID NO: 43 - RNA, 21 nt, synthetic construct
  source 1..21: mol_type = other RNA
gaagaaaaaattctacttcaa

SEQ ID NO: 44 - RNA, 21 nt, synthetic construct
  source 1..21: mol_type = other RNA
aaaaattctacttcaacaaaa

SEQ ID NO: 45 - RNA, 21 nt, synthetic construct
  source 1..21: mol_type = other RNA
aaaattctacttcaacaaaaa

SEQ ID NO: 46 - RNA, 21 nt, synthetic construct
  source 1..21: mol_type = other RNA
ctacttcaacaaaaagtgaaa

SEQ ID NO: 47 - RNA, 21 nt, synthetic construct
  source 1..21: mol_type = other RNA
tacttcaacaaaaagtgaaat

SEQ ID NO: 48 - RNA, 21 nt, synthetic construct
  source 1..21: mol_type = other RNA
ttcaacaaaaagtgaaatatt

SEQ ID NO: 49 - RNA, 21 nt, synthetic construct
  source 1..21: mol_type = other RNA
caacaaaaagtgaaatattta

SEQ ID NO: 50 - RNA, 21 nt, synthetic construct
  source 1..21: mol_type = other RNA
aactccagaacacccagaagt

SEQ ID NO: 51 - RNA, 21 nt, synthetic construct
  source 1..21: mol_type = other RNA
ctccagaacacccagaagtaa

SEQ ID NO: 52 - RNA, 21 nt, synthetic construct
  source 1..21: mol_type = other RNA
acccagaagtaacttcactta

SEQ ID NO: 53 - RNA, 21 nt, synthetic construct
  source 1..21: mol_type = other RNA
ccagaagtaacttcacttaaa

SEQ ID NO: 54 - RNA, 21 nt, synthetic construct
  source 1..21: mol_type = other RNA
cagaagtaacttcacttaaaa

SEQ ID NO: 55 - RNA, 21 nt, synthetic construct
  source 1..21: mol_type = other RNA
aagtaacttcacttaaaactt

SEQ ID NO: 56 - RNA, 21 nt, synthetic construct
  source 1..21: mol_type = other RNA
aacttcacttaaaacttttgt

SEQ ID NO: 57 - RNA, 21 nt, synthetic construct
  source 1..21: mol_type = other RNA
aacttttgtagaaaaacaaga

SEQ ID NO: 58 - RNA, 21 nt, synthetic construct
  source 1..21: mol_type = other RNA
ttgtagaaaaacaagataata

SEQ ID NO: 59 - RNA, 21 nt, synthetic construct
  source 1..21: mol_type = other RNA
aaacaagataatagcatcaaa

SEQ ID NO: 60 - RNA, 21 nt, synthetic construct
  source 1..21: mol_type = other RNA
tagcatcaaagaccttctcca

SEQ ID NO: 61 - RNA, 21 nt, synthetic construct
  source 1..21: mol_type = other RNA
ccagaccgtggaagaccaata

SEQ ID NO: 62 - RNA, 21 nt, synthetic construct
  source 1..21: mol_type = other RNA
cagaccgtggaagaccaatat

SEQ ID NO: 63 - RNA, 21 nt, synthetic construct
  source 1..21: mol_type = other RNA
gaccgtggaagaccaatataa

SEQ ID NO: 64 - RNA, 21 nt, synthetic construct
  source 1..21: mol_type = other RNA
accgtggaagaccaatataaa

SEQ ID NO: 65 - RNA, 21 nt, synthetic construct
  source 1..21: mol_type = other RNA
gtggaagaccaatataaacaa

SEQ ID NO: 66 - RNA, 21 nt, synthetic construct
  source 1..21: mol_type = other RNA
gaagaccaatataaacaatta

SEQ ID NO: 67 - RNA, 21 nt, synthetic construct
  source 1..21: mol_type = other RNA
aagaccaatataaacaattaa

SEQ ID NO: 68 - RNA, 21 nt, synthetic construct
  source 1..21: mol_type = other RNA
aaccaacagcatagtcaaata

SEQ ID NO: 69 - RNA, 21 nt, synthetic construct
  source 1..21: mol_type = other RNA
accaacagcatagtcaaataa

SEQ ID NO: 70 - RNA, 21 nt, synthetic construct
  source 1..21: mol_type = other RNA
tagtcaaataaaagaaataga

SEQ ID NO: 71 - RNA, 21 nt, synthetic construct
  source 1..21: mol_type = other RNA
tcaaataaaagaaatagaaaa

SEQ ID NO: 72 - RNA, 21 nt, synthetic construct
  source 1..21: mol_type = other RNA
atcagctcagaaggactagta

SEQ ID NO: 73 - RNA, 21 nt, synthetic construct
  source 1..21: mol_type = other RNA
cagctcagaaggactagtatt

SEQ ID NO: 74 - RNA, 21 nt, synthetic construct
  source 1..21: mol_type = other RNA
ctcagaaggactagtattcaa

SEQ ID NO: 75 - RNA, 21 nt, synthetic construct
  source 1..21: mol_type = other RNA
cagaaggactagtattcaaga

SEQ ID NO: 76 - RNA, 21 nt, synthetic construct
  source 1..21: mol_type = other RNA
tagtattcaagaacccacaga

SEQ ID NO: 77 - RNA, 21 nt, synthetic construct
  source 1..21: mol_type = other RNA
aacccacagaaatttctctat

SEQ ID NO: 78 - RNA, 21 nt, synthetic construct
  source 1..21: mol_type = other RNA
tccaagccaagagcaccaaga

SEQ ID NO: 79 - RNA, 21 nt, synthetic construct
  source 1..21: mol_type = other RNA
aagccaagagcaccaagaact

SEQ ID NO: 80 - RNA, 21 nt, synthetic construct
  source 1..21: mol_type = other RNA
tactccctttcttcagttgaa

SEQ ID NO: 81 - RNA, 21 nt, synthetic construct
  source 1..21: mol_type = other RNA
tccctttcttcagttgaatga

SEQ ID NO: 82 - RNA, 21 nt, synthetic construct
  source 1..21: mol_type = other RNA
ttcagttgaatgaaataagaa

SEQ ID NO: 83 - RNA, 21 nt, synthetic construct
  source 1..21: mol_type = other RNA
tcagttgaatgaaataagaaa

SEQ ID NO: 84 - RNA, 21 nt, synthetic construct
  source 1..21: mol_type = other RNA
ttgaatgaaataagaaatgta

SEQ ID NO: 85 - RNA, 21 nt, synthetic construct
  source 1..21: mol_type = other RNA
ttcctgctgaatgtaccacca

SEQ ID NO: 86 - RNA, 21 nt, synthetic construct
  source 1..21: mol_type = other RNA
tgctgaatgtaccaccattta

SEQ ID NO: 87 - RNA, 21 nt, synthetic construct
  source 1..21: mol_type = other RNA
ctgaatgtaccaccatttata

SEQ ID NO: 88 - RNA, 21 nt, synthetic construct
  source 1..21: mol_type = other RNA
accaccatttataacagaggt

SEQ ID NO: 89 - RNA, 21 nt, synthetic construct
  source 1..21: mol_type = other RNA
caccatttataacagaggtga

SEQ ID NO: 90 - RNA, 21 nt, synthetic construct
  source 1..21: mol_type = other RNA
accatttataacagaggtgaa

SEQ ID NO: 91 - RNA, 21 nt, synthetic construct
  source 1..21: mol_type = other RNA
aacagaggtgaacatacaagt

SEQ ID NO: 92 - RNA, 21 nt, synthetic construct
  source 1..21: mol_type = other RNA
gaggtgaacatacaagtggca

SEQ ID NO: 93 - RNA, 21 nt, synthetic construct
  source 1..21: mol_type = other RNA
gtgaacatacaagtggcatgt

SEQ ID NO: 94 - RNA, 21 nt, synthetic construct
  source 1..21: mol_type = other RNA
tacaagtggcatgtatgccat

SEQ ID NO: 95 - RNA, 21 nt, synthetic construct
  source 1..21: mol_type = other RNA
ctctcaagtttttcatgtcta

SEQ ID NO: 96 - RNA, 21 nt, synthetic construct
  source 1..21: mol_type = other RNA
ttcatgtctactgtgatgtta

SEQ ID NO: 97 - RNA, 21 nt, synthetic construct
  source 1..21: mol_type = other RNA
catgtctactgtgatgttata

SEQ ID NO: 98 - RNA, 21 nt, synthetic construct
  source 1..21: mol_type = other RNA
gtctactgtgatgttatatca

SEQ ID NO: 99 - RNA, 21 nt, synthetic construct
  source 1..21: mol_type = other RNA
tactgtgatgttatatcaggt

SEQ ID NO: 100 - RNA, 21 nt, synthetic construct
  source 1..21: mol_type = other RNA
caggtagtccatggacattaa

SEQ ID NO: 101 - RNA, 21 nt, synthetic construct
  source 1..21: mol_type = other RNA
ttcaacatcgaatagatggat

SEQ ID NO: 102 - RNA, 21 nt, synthetic construct
  source 1..21: mol_type = other RNA
tagatggatcacaaaacttca

SEQ ID NO: 103 - RNA, 21 nt, synthetic construct
  source 1..21: mol_type = other RNA
tggatcacaaaacttcaatga

SEQ ID NO: 104 - RNA, 21 nt, synthetic construct
  source 1..21: mol_type = other RNA
cacaaaacttcaatgaaacgt

SEQ ID NO: 105 - RNA, 21 nt, synthetic construct
  source 1..21: mol_type = other RNA
atgaaacgtgggagaactaca

SEQ ID NO: 106 - RNA, 21 nt, synthetic construct
  source 1..21: mol_type = other RNA
aacgtgggagaactacaaata

SEQ ID NO: 107 - RNA, 21 nt, synthetic construct
  source 1..21: mol_type = other RNA
gagaactacaaatatggtttt

SEQ ID NO: 108 - RNA, 21 nt, synthetic construct
  source 1..21: mol_type = other RNA
tggaagactggaaagacaaca

SEQ ID NO: 109 - RNA, 21 nt, synthetic construct
  source 1..21: mol_type = other RNA
gaagactggaaagacaacaaa

SEQ ID NO: 110 - RNA, 21 nt, synthetic construct
  source 1..21: mol_type = other RNA
ctggaaagacaacaaacatta

SEQ ID NO: 111 - RNA, 21 nt, synthetic construct
  source 1..21: mol_type = other RNA
tggaaagacaacaaacattat

SEQ ID NO: 112 - RNA, 21 nt, synthetic construct
  source 1..21: mol_type = other RNA
gacaacaaacattatattgaa

SEQ ID NO: 113 - RNA, 21 nt, synthetic construct
  source 1..21: mol_type = other RNA
aacaaacattatattgaatat

SEQ ID NO: 114 - RNA, 21 nt, synthetic construct
  source 1..21: mol_type = other RNA
aacattatattgaatattctt

SEQ ID NO: 115 - RNA, 21 nt, synthetic construct
  source 1..21: mol_type = other RNA
accaactatacgctacatcta

SEQ ID NO: 116 - RNA, 21 nt, synthetic construct
  source 1..21: mol_type = other RNA
tacgctacatctagttgcgat

SEQ ID NO: 117 - RNA, 21 nt, synthetic construct
  source 1..21: mol_type = other RNA
acgctacatctagttgcgatt

SEQ ID NO: 118 - RNA, 21 nt, synthetic construct
  source 1..21: mol_type = other RNA
atctagttgcgattactggca

SEQ ID NO: 119 - RNA, 21 nt, synthetic construct
  source 1..21: mol_type = other RNA
ctggcaatgtccccaatgcaa

SEQ ID NO: 120 - RNA, 21 nt, synthetic construct
  source 1..21: mol_type = other RNA
tccccaatgcaatcccggaaa

SEQ ID NO: 121 - RNA, 21 nt, synthetic construct
  source 1..21: mol_type = other RNA
ccccaatgcaatcccggaaaa

SEQ ID NO: 122 - RNA, 21 nt, synthetic construct
  source 1..21: mol_type = other RNA
cccggaaaacaaagatttggt

SEQ ID NO: 123 - RNA, 21 nt, synthetic construct
  source 1..21: mol_type = other RNA
caaagcaaaaggacacttcaa

SEQ ID NO: 124 - RNA, 21 nt, synthetic construct
  source 1..21: mol_type = other RNA
aagcaaaaggacacttcaact

SEQ ID NO: 125 - RNA, 21 nt, synthetic construct
  source 1..21: mol_type = other RNA
aggacacttcaactgtccaga

SEQ ID NO: 126 - RNA, 21 nt, synthetic construct
  source 1..21: mol_type = other RNA
cacttcaactgtccagagggt

SEQ ID NO: 127 - RNA, 21 nt, synthetic construct
  source 1..21: mol_type = other RNA
ttcaactgtccagagggttat

SEQ ID NO: 128 - RNA, 21 nt, synthetic construct
  source 1..21: mol_type = other RNA
cagagggttattcaggaggct

SEQ ID NO: 129 - RNA, 21 nt, synthetic construct
  source 1..21: mol_type = other RNA
tggtggtggcatgatgagtgt

SEQ ID NO: 130 - RNA, 21 nt, synthetic construct
  source 1..21: mol_type = other RNA
tggtggcatgatgagtgtgga

SEQ ID NO: 131 - RNA, 21 nt, synthetic construct
  source 1..21: mol_type = other RNA
tggcatgatgagtgtggagaa

SEQ ID NO: 132 - RNA, 21 nt, synthetic construct
  source 1..21: mol_type = other RNA
ggcatgatgagtgtggagaaa

SEQ ID NO: 133 - RNA, 21 nt, synthetic construct
  source 1..21: mol_type = other RNA
atggtaaatataacaaaccaa

SEQ ID NO: 134 - RNA, 21 nt, synthetic construct
  source 1..21: mol_type = other RNA
acaaaccaagagcaaaatcta

SEQ ID NO: 135 - RNA, 21 nt, synthetic construct
  source 1..21: mol_type = other RNA
caaaccaagagcaaaatctaa

SEQ ID NO: 136 - RNA, 21 nt, synthetic construct
  source 1..21: mol_type = other RNA
agccagagaggagaagaggat

SEQ ID NO: 137 - RNA, 21 nt, synthetic construct
  source 1..21: mol_type = other RNA
ccagagaggagaagaggatta

SEQ ID NO: 138 - RNA, 21 nt, synthetic construct
  source 1..21: mol_type = other RNA
cagagaggagaagaggattat

SEQ ID NO: 139 - RNA, 21 nt, synthetic construct
  source 1..21: mol_type = other RNA
gagaggagaagaggattatct

SEQ ID NO: 140 - RNA, 21 nt, synthetic construct
  source 1..21: mol_type = other RNA
gagaagaggattatcttggaa

SEQ ID NO: 141 - RNA, 21 nt, synthetic construct
  source 1..21: mol_type = other RNA
aagtctcaaaatggaaggtta

SEQ ID NO: 142 - RNA, 21 nt, synthetic construct
  source 1..21: mol_type = other RNA
gtctcaaaatggaaggttata

SEQ ID NO: 143 - RNA, 21 nt, synthetic construct
  source 1..21: mol_type = other RNA
aaaatggaaggttatactcta

SEQ ID NO: 144 - RNA, 21 nt, synthetic construct
  source 1..21: mol_type = other RNA
atggaaggttatactctataa

SEQ ID NO: 145 - RNA, 21 nt, synthetic construct
  source 1..21: mol_type = other RNA
tggaaggttatactctataaa

SEQ ID NO: 146 - RNA, 21 nt, synthetic construct
  source 1..21: mol_type = other RNA
aggttatactctataaaatca

SEQ ID NO: 147 - RNA, 21 nt, synthetic construct
  source 1..21: mol_type = other RNA
atgttgatccatccaacagat

SEQ ID NO: 148 - RNA, 21 nt, synthetic construct
  source 1..21: mol_type = other RNA
ttgatccatccaacagattca

SEQ ID NO: 149 - RNA, 21 nt, synthetic construct
  source 1..21: mol_type = other RNA
atccatccaacagattcagaa

SEQ ID NO: 150 - RNA, 21 nt, synthetic construct
  source 1..21: mol_type = other RNA
tccatccaacagattcagaaa

SEQ ID NO: 151 - RNA, 21 nt, synthetic construct
  source 1..21: mol_type = other RNA
atccaacagattcagaaagct

SEQ ID NO: 152 - RNA, 21 nt, synthetic construct
  source 1..21: mol_type = other RNA
tccaacagattcagaaagctt

SEQ ID NO: 153 - RNA, 21 nt, synthetic construct
  source 1..21: mol_type = other RNA
aacagattcagaaagctttga

SEQ ID NO: 154 - RNA, 21 nt, synthetic construct
  source 1..21: mol_type = other RNA
cagattcagaaagctttgaat

SEQ ID NO: 155 - RNA, 21 nt, synthetic construct
  source 1..21: mol_type = other RNA
attgtcttgatcaattctgga

SEQ ID NO: 156 - RNA, 21 nt, synthetic construct
  source 1..21: mol_type = other RNA
ttaagtgaagttacttctggg

SEQ ID NO: 157 - RNA, 21 nt, synthetic construct
  source 1..21: mol_type = other RNA
tctacaaaagttttaagtgaa

SEQ ID NO: 158 - RNA, 21 nt, synthetic construct
  source 1..21: mol_type = other RNA
tttctacaaaagttttaagtg

SEQ ID NO: 159 - RNA, 21 nt, synthetic construct
  source 1..21: mol_type = other RNA
tgaagaaagggagtagttctt

SEQ ID NO: 160 - RNA, 21 nt, synthetic construct
  source 1..21: mol_type = other RNA
taacatcacagtagacatgaa

SEQ ID NO: 161 - RNA, 21 nt, synthetic construct
  source 1..21: mol_type = other RNA
taaaacataattagattgctt

SEQ ID NO: 162 - RNA, 21 nt, synthetic construct
  source 1..21: mol_type = other RNA
attcgtaaaacataattagat

SEQ ID NO: 163 - RNA, 21 nt, synthetic construct
  source 1..21: mol_type = other RNA
agatgtagcgtatagttggtt

SEQ ID NO: 164 - RNA, 21 nt, synthetic construct
  source 1..21: mol_type = other RNA
aacaataaaaagaaggagctt

SEQ ID NO: 165 - RNA, 21 nt, synthetic construct
  source 1..21: mol_type = other RNA
tggaggaaataactagaggaa

SEQ ID NO: 166 - RNA, 21 nt, synthetic construct
  source 1..21: mol_type = other RNA
ttctggaggaaataactagag

SEQ ID NO: 167 - RNA, 21 nt, synthetic construct
  source 1..21: mol_type = other RNA
tcttgatcaattctggaggaa

SEQ ID NO: 168 - RNA, 21 nt, synthetic construct
  source 1..21: mol_type = other RNA
ttgtcttgatcaattctggag

SEQ ID NO: 169 - RNA, 21 nt, synthetic construct
  source 1..21: mol_type = other RNA
attgtcttgatcaattctgga

SEQ ID NO: 170 - RNA, 21 nt, synthetic construct
  source 1..21: mol_type = other RNA
atgatgaattgtcttgatcaa

SEQ ID NO: 171 - RNA, 21 nt, synthetic construct
  source 1..21: mol_type = other RNA
aatcaaatgatgaattgtctt

SEQ ID NO: 172 - RNA, 21 nt, synthetic construct
  source 1..21: mol_type = other RNA
agaatcaaatgatgaattgtc

SEQ ID NO: 173 - RNA, 21 nt, synthetic construct
  source 1..21: mol_type = other RNA
taaaatttttacatcgtctaa

SEQ ID NO: 174 - RNA, 21 nt, synthetic construct
  source 1..21: mol_type = other RNA
tttggcccttcgtcttatgga

SEQ ID NO: 175 - RNA, 21 nt, synthetic construct
  source 1..21: mol_type = other RNA
tcattaatttggcccttcgtc

SEQ ID NO: 176 - RNA, 21 nt, synthetic construct
  source 1..21: mol_type = other RNA
tgagtttttgaaatatgtcat

SEQ ID NO: 177 - RNA, 21 nt, synthetic construct
  source 1..21: mol_type = other RNA
ttgagtttttgaaatatgtca

SEQ ID NO: 178 - RNA, 21 nt, synthetic construct
  source 1..21: mol_type = other RNA
tctccacactcatcatgccac

SEQ ID NO: 179 - RNA, 21 nt, synthetic construct
  source 1..21: mol_type = other RNA
atagatcataaaaagactgat

SEQ ID NO: 180 - RNA, 21 nt, synthetic construct
  source 1..21: mol_type = other RNA
tgatttcactggtttgcagcg

SEQ ID NO: 181 - RNA, 21 nt, synthetic construct
  source 1..21: mol_type = other RNA
tttgatttcactggtttgcag

SEQ ID NO: 182 - RNA, 21 nt, synthetic construct
  source 1..21: mol_type = other RNA
tcttctttgatttcactggtt

SEQ ID NO: 183 - RNA, 21 nt, synthetic construct
  source 1..21: mol_type = other RNA
ttcttctttgatttcactggt

SEQ ID NO: 184 - RNA, 21 nt, synthetic construct
  source 1..21: mol_type = other RNA
tcttcttctttgatttcactg

SEQ ID NO: 185 - RNA, 21 nt, synthetic construct
  source 1..21: mol_type = other RNA
ttttcttcttctttgatttca

SEQ ID NO: 186 - RNA, 21 nt, synthetic construct
  source 1..21: mol_type = other RNA
ttccttttcttcttctttgat

SEQ ID NO: 187 - RNA, 21 nt, synthetic construct
  source 1..21: mol_type = other RNA
ttctcagttccttttcttctt

SEQ ID NO: 188 - RNA, 21 nt, synthetic construct
  source 1..21: mol_type = other RNA
ttcttctcagttccttttctt

SEQ ID NO: 189 - RNA, 21 nt, synthetic construct
  source 1..21: mol_type = other RNA
tgtagttcttctcagttcctt

SEQ ID NO: 190 - RNA, 21 nt, synthetic construct
  source 1..21: mol_type = other RNA
atgtagttcttctcagttcct

SEQ ID NO: 191 - RNA, 21 nt, synthetic construct
  source 1..21: mol_type = other RNA
tatatgtagttcttctcagtt

SEQ ID NO: 192 - RNA, 21 nt, synthetic construct
  source 1..21: mol_type = other RNA
ttatatgtagttcttctcagt

SEQ ID NO: 193 - RNA, 21 nt, synthetic construct
  source 1..21: mol_type = other RNA
tacctcttcatttttgacttg

SEQ ID NO: 194 - RNA, 21 nt, synthetic construct
  source 1..21: mol_type = other RNA
acatattctttacctcttcat

SEQ ID NO: 195 - RNA, 21 nt, synthetic construct
  source 1..21: mol_type = other RNA
tgagttgagttcaagtgacat

SEQ ID NO: 196 - RNA, 21 nt, synthetic construct
  source 1..21: mol_type = other RNA
tagaattttttcttctaggag

SEQ ID NO: 197 - RNA, 21 nt, synthetic construct
  source 1..21: mol_type = other RNA
ttgaagtagaattttttcttc

SEQ ID NO: 198 - RNA, 21 nt, synthetic construct
  source 1..21: mol_type = other RNA
ttttgttgaagtagaattttt

SEQ ID NO: 199 - RNA, 21 nt, synthetic construct
  source 1..21: mol_type = other RNA
tttttgttgaagtagaatttt

SEQ ID NO: 200 - RNA, 21 nt, synthetic construct
  source 1..21: mol_type = other RNA
tttcactttttgttgaagtag

SEQ ID NO: 201 - RNA, 21 nt, synthetic construct
  source 1..21: mol_type = other RNA
atttcactttttgttgaagta

SEQ ID NO: 202 - RNA, 21 nt, synthetic construct
  source 1..21: mol_type = other RNA
aatatttcactttttgttgaa

SEQ ID NO: 203 - RNA, 21 nt, synthetic construct
  source 1..21: mol_type = other RNA
taaatatttcactttttgttg

SEQ ID NO: 204 - RNA, 21 nt, synthetic construct
  source 1..21: mol_type = other RNA
acttctgggtgttctggagtt

SEQ ID NO: 205 - RNA, 21 nt, synthetic construct
  source 1..21: mol_type = other RNA
ttacttctgggtgttctggag

SEQ ID NO: 206 - RNA, 21 nt, synthetic construct
  source 1..21: mol_type = other RNA
taagtgaagttacttctgggt

SEQ ID NO: 207 - RNA, 21 nt, synthetic construct
  source 1..21: mol_type = other RNA
tttaagtgaagttacttctgg

SEQ ID NO: 208 - RNA, 21 nt, synthetic construct
  source 1..21: mol_type = other RNA
ttttaagtgaagttacttctg

SEQ ID NO: 209 - RNA, 21 nt, synthetic construct
  source 1..21: mol_type = other RNA
aagttttaagtgaagttactt

SEQ ID NO: 210 - RNA, 21 nt, synthetic construct
  source 1..21: mol_type = other RNA
acaaaagttttaagtgaagtt

SEQ ID NO: 211 - RNA, 21 nt, synthetic construct
  source 1..21: mol_type = other RNA
tcttgtttttctacaaaagtt

SEQ ID NO: 212 - RNA, 21 nt, synthetic construct
  source 1..21: mol_type = other RNA
tattatcttgtttttctacaa

SEQ ID NO: 213 - RNA, 21 nt, synthetic construct
  source 1..21: mol_type = other RNA
tttgatgctattatcttgttt

SEQ ID NO: 214 - RNA, 21 nt, synthetic construct
  source 1..21: mol_type = other RNA
tggagaaggtctttgatgcta

SEQ ID NO: 215 - RNA, 21 nt, synthetic construct
  source 1..21: mol_type = other RNA
tattggtcttccacggtctgg

SEQ ID NO: 216 - RNA, 21 nt, synthetic construct
  source 1..21: mol_type = other RNA
atattggtcttccacggtctg

SEQ ID NO: 217 - RNA, 21 nt, synthetic construct
  source 1..21: mol_type = other RNA
ttatattggtcttccacggtc

SEQ ID NO: 218 - RNA, 21 nt, synthetic construct
  source 1..21: mol_type = other RNA
tttatattggtcttccacggt

SEQ ID NO: 219 - RNA, 21 nt, synthetic construct
  source 1..21: mol_type = other RNA
ttgtttatattggtcttccac

SEQ ID NO: 220 - RNA, 21 nt, synthetic construct
  source 1..21: mol_type = other RNA
taattgtttatattggtcttc

SEQ ID NO: 221 - RNA, 21 nt, synthetic construct
  source 1..21: mol_type = other RNA
ttaattgtttatattggtctt

SEQ ID NO: 222 - RNA, 21 nt, synthetic construct
  source 1..21: mol_type = other RNA
tatttgactatgctgttggtt

SEQ ID NO: 223 - RNA, 21 nt, synthetic construct
  source 1..21: mol_type = other RNA
ttatttgactatgctgttggt

SEQ ID NO: 224 - RNA, 21 nt, synthetic construct
  source 1..21: mol_type = other RNA
tctatttcttttatttgacta

SEQ ID NO: 225 - RNA, 21 nt, synthetic construct
  source 1..21: mol_type = other RNA
ttttctatttcttttatttga

SEQ ID NO: 226 - RNA, 21 nt, synthetic construct
  source 1..21: mol_type = other RNA
tactagtccttctgagctgat

SEQ ID NO: 227 - RNA, 21 nt, synthetic construct
  source 1..21: mol_type = other RNA
aatactagtccttctgagctg

SEQ ID NO: 228 - RNA, 21 nt, synthetic construct
  source 1..21: mol_type = other RNA
ttgaatactagtccttctgag

SEQ ID NO: 229 - RNA, 21 nt, synthetic construct
  source 1..21: mol_type = other RNA
tcttgaatactagtccttctg

SEQ ID NO: 230 - RNA, 21 nt, synthetic construct
  source 1..21: mol_type = other RNA
tctgtgggttcttgaatacta

SEQ ID NO: 231 - RNA, 21 nt, synthetic construct
  source 1..21: mol_type = other RNA
atagagaaatttctgtgggtt

SEQ ID NO: 232 - RNA, 21 nt, synthetic construct
  source 1..21: mol_type = other RNA
tcttggtgctcttggcttgga

SEQ ID NO: 233 - RNA, 21 nt, synthetic construct
  source 1..21: mol_type = other RNA
agttcttggtgctcttggctt

SEQ ID NO: 234 - RNA, 21 nt, synthetic construct
  source 1..21: mol_type = other RNA
ttcaactgaagaaagggagta

SEQ ID NO: 235 - RNA, 21 nt, synthetic construct
  source 1..21: mol_type = other RNA
tcattcaactgaagaaaggga

SEQ ID NO: 236 - RNA, 21 nt, synthetic construct
  source 1..21: mol_type = other RNA
ttcttatttcattcaactgaa

SEQ ID NO: 237 - RNA, 21 nt, synthetic construct
  source 1..21: mol_type = other RNA
tttcttatttcattcaactga

SEQ ID NO: 238 - RNA, 21 nt, synthetic construct
  source 1..21: mol_type = other RNA
tacatttcttatttcattcaa

SEQ ID NO: 239 - RNA, 21 nt, synthetic construct
  source 1..21: mol_type = other RNA
tggtggtacattcagcaggaa

SEQ ID NO: 240 - RNA, 21 nt, synthetic construct
  source 1..21: mol_type = other RNA
taaatggtggtacattcagca

SEQ ID NO: 241 - RNA, 21 nt, synthetic construct
  source 1..21: mol_type = other RNA
tataaatggtggtacattcag

SEQ ID NO: 242 - RNA, 21 nt, synthetic construct
  source 1..21: mol_type = other RNA
acctctgttataaatggtggt

SEQ ID NO: 243 - RNA, 21 nt, synthetic construct
  source 1..21: mol_type = other RNA
tcacctctgttataaatggtg

SEQ ID NO: 244 - RNA, 21 nt, synthetic construct
  source 1..21: mol_type = other RNA
ttcacctctgttataaatggt

SEQ ID NO: 245 - RNA, 21 nt, synthetic construct
  source 1..21: mol_type = other RNA
acttgtatgttcacctctgtt

SEQ ID NO: 246 - RNA, 21 nt, synthetic construct
  source 1..21: mol_type = other RNA
tgccacttgtatgttcacctc

SEQ ID NO: 247 - RNA, 21 nt, synthetic construct
  source 1..21: mol_type = other RNA
acatgccacttgtatgttcac

SEQ ID NO: 248 - RNA, 21 nt, synthetic construct
  source 1..21: mol_type = other RNA
atggcatacatgccacttgta

SEQ ID NO: 249 - RNA, 21 nt, synthetic construct
  source 1..21: mol_type = other RNA
tagacatgaaaaacttgagag

SEQ ID NO: 250 - RNA, 21 nt, synthetic construct
  source 1..21: mol_type = other RNA
taacatcacagtagacatgaa

SEQ ID NO: 251 - RNA, 21 nt, synthetic construct
  source 1..21: mol_type = other RNA
tataacatcacagtagacatg

SEQ ID NO: 252 - RNA, 21 nt, synthetic construct
  source 1..21: mol_type = other RNA
tgatataacatcacagtagac

SEQ ID NO: 253 - RNA, 21 nt, synthetic construct
  source 1..21: mol_type = other RNA
acctgatataacatcacagta

SEQ ID NO: 254 - RNA, 21 nt, synthetic construct
  source 1..21: mol_type = other RNA
ttaatgtccatggactacctg

SEQ ID NO: 255 - RNA, 21 nt, synthetic construct
  source 1..21: mol_type = other RNA
atccatctattcgatgttgaa

SEQ ID NO: 256 - RNA, 21 nt, synthetic construct
  source 1..21: mol_type = other RNA
tgaagttttgtgatccatcta

SEQ ID NO: 257 - RNA, 21 nt, synthetic construct
  source 1..21: mol_type = other RNA
tcattgaagttttgtgatcca

SEQ ID NO: 258 - RNA, 21 nt, synthetic construct
  source 1..21: mol_type = other RNA
acgtttcattgaagttttgtg

SEQ ID NO: 259 - RNA, 21 nt, synthetic construct
  source 1..21: mol_type = other RNA
tgtagttctcccacgtttcat

SEQ ID NO: 260 - RNA, 21 nt, synthetic construct
  source 1..21: mol_type = other RNA
tatttgtagttctcccacgtt

SEQ ID NO: 261 - RNA, 21 nt, synthetic construct
  source 1..21: mol_type = other RNA
aaaaccatatttgtagttctc

SEQ ID NO: 262 - RNA, 21 nt, synthetic construct
  source 1..21: mol_type = other RNA
tgttgtctttccagtcttcca

SEQ ID NO: 263 - RNA, 21 nt, synthetic construct
  source 1..21: mol_type = other RNA
tttgttgtctttccagtcttc

SEQ ID NO: 264 - RNA, 21 nt, synthetic construct
  source 1..21: mol_type = other RNA
taatgtttgttgtctttccag

SEQ ID NO: 265 - RNA, 21 nt, synthetic construct
  source 1..21: mol_type = other RNA
ataatgtttgttgtctttcca

SEQ ID NO: 266 - RNA, 21 nt, synthetic construct
  source 1..21: mol_type = other RNA
ttcaatataatgtttgttgtc

SEQ ID NO: 267 - RNA, 21 nt, synthetic construct
  source 1..21: mol_type = other RNA
atattcaatataatgtttgtt

SEQ ID NO: 268 - RNA, 21 nt, synthetic construct
  source 1..21: mol_type = other RNA
aagaatattcaatataatgtt

SEQ ID NO: 269 - RNA, 21 nt, synthetic construct
  source 1..21: mol_type = other RNA
tagatgtagcgtatagttggt

SEQ ID NO: 270 - RNA, 21 nt, synthetic construct
  source 1..21: mol_type = other RNA
atcgcaactagatgtagcgta

SEQ ID NO: 271 - RNA, 21 nt, synthetic construct
  source 1..21: mol_type = other RNA
aatcgcaactagatgtagcgt

SEQ ID NO: 272 - RNA, 21 nt, synthetic construct
  source 1..21: mol_type = other RNA
tgccagtaatcgcaactagat

SEQ ID NO: 273 - RNA, 21 nt, synthetic construct
  source 1..21: mol_type = other RNA
ttgcattggggacattgccag

SEQ ID NO: 274 - RNA, 21 nt, synthetic construct
  source 1..21: mol_type = other RNA
tttccgggattgcattgggga

SEQ ID NO: 275 - RNA, 21 nt, synthetic construct
  source 1..21: mol_type = other RNA
ttttccgggattgcattgggg

SEQ ID NO: 276 - RNA, 21 nt, synthetic construct
  source 1..21: mol_type = other RNA
accaaatctttgttttccggg

SEQ ID NO: 277 - RNA, 21 nt, synthetic construct
  source 1..21: mol_type = other RNA
ttgaagtgtccttttgctttg

SEQ ID NO: 278 - RNA, 21 nt, synthetic construct
  source 1..21: mol_type = other RNA
agttgaagtgtccttttgctt

SEQ ID NO: 279 - RNA, 21 nt, synthetic construct
  source 1..21: mol_type = other RNA
tctggacagttgaagtgtcct

SEQ ID NO: 280 - RNA, 21 nt, synthetic construct
  source 1..21: mol_type = other RNA
accctctggacagttgaagtg

SEQ ID NO: 281 - RNA, 21 nt, synthetic construct
  source 1..21: mol_type = other RNA
ataaccctctggacagttgaa

SEQ ID NO: 282 - RNA, 21 nt, synthetic construct
  source 1..21: mol_type = other RNA
agcctcctgaataaccctctg

SEQ ID NO: 283 - RNA, 21 nt, synthetic construct
  source 1..21: mol_type = other RNA
acactcatcatgccaccacca

SEQ ID NO: 284 - RNA, 21 nt, synthetic construct
  source 1..21: mol_type = other RNA
tccacactcatcatgccacca

SEQ ID NO: 285 - RNA, 21 nt, synthetic construct
  source 1..21: mol_type = other RNA
ttctccacactcatcatgcca

SEQ ID NO: 286 - RNA, 21 nt, synthetic construct
  source 1..21: mol_type = other RNA
tttctccacactcatcatgcc

SEQ ID NO: 287 - RNA, 21 nt, synthetic construct
  source 1..21: mol_type = other RNA
ttggtttgttatatttaccat

SEQ ID NO: 288 - RNA, 21 nt, synthetic construct
  source 1..21: mol_type = other RNA
tagattttgctcttggtttgt

SEQ ID NO: 289 - RNA, 21 nt, synthetic construct
  source 1..21: mol_type = other RNA
ttagattttgctcttggtttg

SEQ ID NO: 290 - RNA, 21 nt, synthetic construct
  source 1..21: mol_type = other RNA
atcctcttctcctctctggct

SEQ ID NO: 291 - RNA, 21 nt, synthetic construct
  source 1..21: mol_type = other RNA
taatcctcttctcctctctgg

SEQ ID NO: 292 - RNA, 21 nt, synthetic construct
  source 1..21: mol_type = other RNA
ataatcctcttctcctctctg

SEQ ID NO: 293 - RNA, 21 nt, synthetic construct
  source 1..21: mol_type = other RNA
agataatcctcttctcctctc

SEQ ID NO: 294 - RNA, 21 nt, synthetic construct
  source 1..21: mol_type = other RNA
ttccaagataatcctcttctc

SEQ ID NO: 295 - RNA, 21 nt, synthetic construct
  source 1..21: mol_type = other RNA
taaccttccattttgagactt

SEQ ID NO: 296 - RNA, 21 nt, synthetic construct
  source 1..21: mol_type = other RNA
tataaccttccattttgagac

SEQ ID NO: 297 - RNA, 21 nt, synthetic construct
  source 1..21: mol_type = other RNA
tagagtataaccttccatttt

SEQ ID NO: 298 - RNA, 21 nt, synthetic construct
  source 1..21: mol_type = other RNA
ttatagagtataaccttccat

SEQ ID NO: 299 - RNA, 21 nt, synthetic construct
  source 1..21: mol_type = other RNA
tttatagagtataaccttcca

SEQ ID NO: 300 - RNA, 21 nt, synthetic construct
  source 1..21: mol_type = other RNA
tgattttatagagtataacct

SEQ ID NO: 301 - RNA, 21 nt, synthetic construct
  source 1..21: mol_type = other RNA
atctgttggatggatcaacat

SEQ ID NO: 302 - RNA, 21 nt, synthetic construct
  source 1..21: mol_type = other RNA
tgaatctgttggatggatcaa

SEQ ID NO: 303 - RNA, 21 nt, synthetic construct
  source 1..21: mol_type = other RNA
ttctgaatctgttggatggat

SEQ ID NO: 304 - RNA, 21 nt, synthetic construct
  source 1..21: mol_type = other RNA
tttctgaatctgttggatgga

SEQ ID NO: 305 - RNA, 21 nt, synthetic construct
  source 1..21: mol_type = other RNA
agctttctgaatctgttggat

SEQ ID NO: 306 - RNA, 21 nt, synthetic construct
  source 1..21: mol_type = other RNA
aagctttctgaatctgttgga

SEQ ID NO: 307 - RNA, 21 nt, synthetic construct
  source 1..21: mol_type = other RNA
tcaaagctttctgaatctgtt

SEQ ID NO: 308 - RNA, 21 nt, synthetic construct
  source 1..21: mol_type = other RNA
attcaaagctttctgaatctg

SEQ ID NO: 309 - RNA, 19 nt, synthetic construct
  source 1..19: mol_type = other RNA
ttccgaacgtgtcacgttt

SEQ ID NO: 310 - RNA, 21 nt, synthetic construct
  source 1..21: mol_type = other RNA
acgtgacacgttcggagaatt